(19) United States
(12) Patent Application Publication
(10) Pub. No.: US 2004/0248942 A1
(43) Pub. Date: Dec. 9, 2004

(54) Novel formulation, omeprazole antacid complex-immediate release for rapid and sustained suppression of gastric acid

(76) Inventors: Bonnie Hepburn, Escondido, CA (US); Barry Goldlust, San Diego, CA (US)

Correspondence Address: WILSON SONSINI GOODRICH & ROSATI, 650 PAGE MILL ROAD, PALO ALTO, CA, 943041050

(21) Appl. No.: 10/783,871

(22) Filed: Feb. 20, 2004

Related U.S. Application Data
(60) Provisional application No. 60/448,627, filed on Feb. 20, 2003.

(51) Int. Cl. A61K 31/4439

(52) U.S. Cl. 514/338

(57) ABSTRACT

The present invention is directed to methods, kits, combinations, and compositions for treating, preventing or reducing the risk of developing a gastrointestinal disorder or disease, or the symptoms associated with, or related to a gastrointestinal disorder or disease in a subject in need thereof. In one aspect, the present invention provides a pharmaceutical composition comprising a proton pump inhibiting agent and a buffering agent for oral administration and ingestion by a subject. Upon administration, the composition contacts the gastric fluid of the stomach and increases the gastric fluid pH of the stomach to a pH that substantially prevents or inhibits acid degradation of the proton pump inhibiting agent in the gastric fluid and allows a measurable serum concentration of the proton pump inhibiting agent to be absorbed into the blood serum of the subject.

### RELATED APPLICATIONS

This application claims priority to U.S. Provisional Application No. 60/448,627, filed Feb. 20, 2003.

### TECHNICAL FIELD

The present invention relates to combinations of a proton pump inhibiting agent and a buffering agent that have been found to possess improved bioavailability, chemical stability, physical stability, dissolution profiles, disintegration times, safety, as well as other improved pharmacokinetic, pharmacodynamic, chemical and/or physical properties. The present invention is directed to methods, kits, combinations, and compositions for treating, preventing or reducing the risk of developing a gastrointestinal disorder or disease, or the symptoms associated with, or related to, a gastrointestinal disorder or disease in a subject in need thereof.

### BACKGROUND OF THE INVENTION

Omeprazole is a substituted benzimidazole, 5-methoxy-2-[(4-methoxy-3,5-dimethyl-2-pyridinyl)methyl]sulfinyl]-1H-benzimidazole, that inhibits gastric acid secretion. Omeprazole belongs to a class of antisecretory compounds called proton pump inhibiting agents (“PPIs”) that do not exhibit anti-cholinergic or H₂ histamine antagonist properties. Drugs of this class suppress gastric acid secretion by the specific inhibition of the H⁺, K⁺-ATPase proton pump at the secretory surface of the gastric parietal cell.

Typically, omeprazole, lansoprazole and other proton pump inhibitors are formulated in an enteric-coated solid dosage form (as either a delayed-release capsule or tablet) or as an intravenous solution (as a product for reconstitution), and are prescribed for short-term treatment of active duodenal ulcers, gastric ulcers, gastroesophageal reflux disease (GERD), severe erosive esophagitis, poorly responsive symptomatic gastroesophageal reflux disease, and pathological hypersecretory conditions such as Zollinger Ellison syndrome. These conditions are caused by an imbalance between acid and pepsin production, called aggressive factors, and mucous, bicarbonate and prostaglandin production, called defensive factors. These above-listed conditions commonly arise in healthy or critically ill patients, and may be accompanied by significant upper gastrointestinal bleeding.

H₂-antagonists, antacids, and sucralfate are commonly administered to minimize the pain and the complications related to these conditions. These drugs have certain disadvantages associated with their use. Some of these drugs are not completely effective in the treatment of the aforementioned conditions and/or produce adverse side effects, such as mental confusion, constipation, diarrhea, and thrombocytopenia. H₂-antagonists, such as ranitidine and cimetidine, are relatively costly modes of therapy, particularly in NPO patients, which frequently require the use of automated infusion pumps for continuous intravenous infusion of the drug.

It is believed that omeprazole (Prilosec®), lansoprazole (Prevacid®), and other proton pump inhibitors reduce gastric acid production by inhibiting H⁺,K⁺-ATPase of the parietal cell—the final common pathway for gastric acid secretion (Fellenius et al., Substituted Benzimidazoles Inhibit Gastric Acid Secretion by Blocking H⁺,K⁺-ATPase, Nature, 290: 159-161 (1981); Wallmark et al., The Relationship Between Gastric Acid Secretion and Gastric H⁺,K⁺-ATPase Activity, J. Biol.Chem., 260: 13681-13684 (1985); Fryklund et al., Function and Structure of Parietal Cells After H⁺,K⁺-ATPase Blockade, Am. J. Physiol., 254 (3 pt 1); G399-407 (1988)). Some proton pump inhibitors contain a sulfinyl group in a bridge between substituted benzimidazole and a pyridine, as illustrated below.

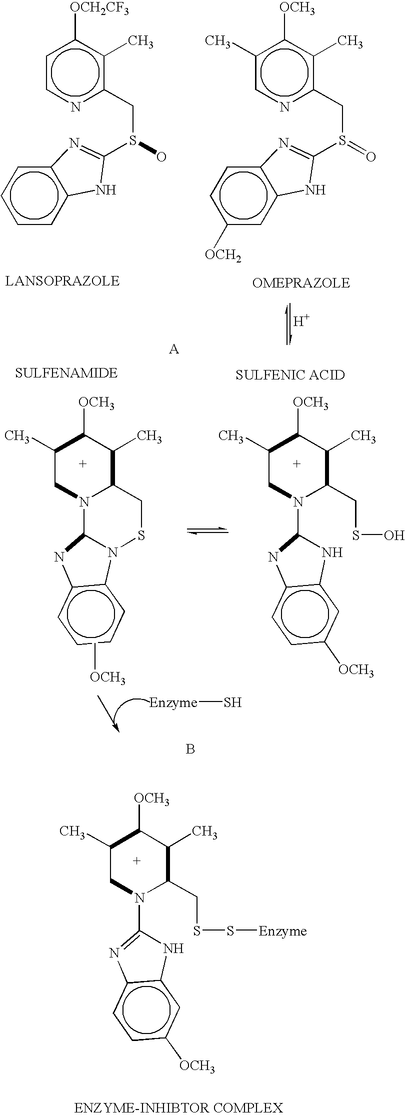

At neutral pH, omeprazole, lansoprazole and other proton pump inhibitors are chemically stable, lipid-soluble, weak bases that are devoid of inhibitory activity. When delivered in an enteric-coated form, these neutral weak bases are believed to reach parietal cells from the blood and diffuse into the secretory canaliculi, where the drugs become protonated and thereby trapped. The protonated agent rearranges to form a sulfenic acid and a sulfenamide. The sulfenamide interacts covalently with sulfhydryl groups at critical sites in the extracellular (luminal) domain of the membrane-spanning H⁺,K⁺-ATPase (Hardman et al., Goodman & Gilman's The Pharmacological Basis of Therapeutics, p. 907 (9^{th} ed. 1996)). Omeprazole and lansoprazole, therefore, are prodrugs that must be activated to be effective. The specificity of the effects of proton pump inhibitors is also dependent upon: (a) the selective distribution of H⁺,K⁺-ATPase; (b) the requirement for acidic conditions to catalyze generation of the reactive inhibitor; and (c) the trapping of the protonated drug and the cationic sulfenamide within the acidic canaliculi and adjacent to the target enzyme. (Hardman et al., 1996).

Proton pump inhibitors are acid labile and therefore have been formulated as enteric-coated dosage forms to prevent acid degradation. Examples include, omeprazole (Prilosec®), lansoprazole (Prevacid®), esomeprazole (Nexium®), rabeprazole (Aciphex®), pantoprazole (Protonix®), pariprazole and leminoprazole. Prilosec® (omeprazole) is formulated as enteric-coated granules in gelatin capsules. Prevacid® (lansoprazole) is available as enteric-coated granules in gelatin capsules, and as enteric-coated microspheres for use as a liquid suspension. Nexium® (esomeprazole magnesium) is enteric-coated granules in gelatin capsules. Although these drugs are stable at alkaline pH, they are destroyed rapidly as pH falls (for example, by gastric acid). Therefore, if the enteric-coating is disrupted (for example, through trituration to compound a liquid or by chewing), the dosage forms of the prior art will be exposed to degradation by the gastric acid in the stomach.

Upon ingestion, an acid-labile pharmaceutical compound must be protected from contact with acidic stomach secretions to maintain its pharmaceutical activity. Thus, compositions with enteric-coatings have been designed to dissolve at a pH to ensure that the drug is released in the proximal region of the small intestine (duodenum), not in the stomach. However, due to their pH-dependent attributes and the uncertainty of gastric retention time, in-vivo performance as well as inter- and intra-subject variability are major issues for using enteric-coated systems for controlled release of a drug.

To ensure that enteric-coatings dissolve or disintegrate rapidly at the target intestine site, which is near a neutral pH, enteric-coatings have been designed to generally dissolve at about pH 5. However, at this pH, most acid-labile pharmaceutical agents are still susceptible to acid degradation depending on the particular pKa of the agent. As an acid-labile compound upon ingestion must be transferred in intact form, i.e., a non-acid degraded or reacted form, to the duodenum where the pH is near or above its pKa, the enteric-coating must be resistant to dissolution and disintegration in the stomach, that is, be impermeable to gastric fluids while residing in the stomach.

Additionally, the therapeutic onset of an enteric-coated dosage form is largely dependent upon gastric emptying time. In most subjects, gastric emptying is generally an all or nothing process, and generally varies from about 30 minutes to several hours after ingestion. Thus, for a period of time following ingestion, an enteric-coated dosage form resides in the low pH environment of the stomach before moving into the duodenum. During this time, the enteric-coating may begin to dissolve, or imperfections or cracks in the coating may develop, allowing gastric acid to penetrate the coating and prematurely release drug into the stomach rather than in the small intestine. In the absence of buffering agent, an acid-labile drug that is exposed to this gastric acid is rapidly degraded and rendered therapeutically ineffective.

Enteric-coated dosage forms are also generally taken on an empty stomach with a glass of water. This minimizes exposure time to gastric fluid, as it ensure gastric emptying within about 30 minutes or so, and delivery of the dosage form from the stomach to the duodenum. Once in the duodenum, optimal conditions exist for the enteric-coating to dissolve and release the drug into the bloodstream where absorption of a non-acid degraded drug occurs.

If food is ingested contemporaneously with the administration of an enteric-coated dosage form, gastric emptying may not only be slowed, but there is also an increases in the pH of the stomach from about pH 1 to about 5 over the next several hours, depending on, for example, the general health of the subject and the composition being administered. When the pH begins to approach 5, the enteric-coating begins to dissolve away resulting in premature release of the drug into the stomach. This is a particular problem in the elderly who already have elevated gastric acid pH, as there is a general decline in gastric acid secretion in the stomach as one ages. Also, when the ingested food contains any fat, gastric emptying can be delayed for up to 3 to 6 hours or more, as fat in any form combined with bile and pancreatic fluids strongly inhibits gastric emptying. Thus, as a general rule, enteric-coated dosage forms should only be ingested on an empty stomach with a glass of water to provide optimal conditions for dissolution and absorption.

Furthermore, the effects of the currently marketed delayed-release enteric-coated proton pump inhibitor formulations may not be seen until several hours after dosing, necessitating administration of the enteric-coated formulation to a patient several hours prior to ingesting a meal (e.g., to a “fasting” patient) for the patient to experience relief of gastrointestinal symptoms that arise upon eating. Thus, administration of a delayed-release formulation to a patient either with food or after initiating ingestion of a meal (e.g., to a “fed” patient) will not result in any immediate relief from food-induced symptoms, and in fact, may result in the continuation of patient suffering for several hours after ingestion of the offending meal. In addition, a patient may not always anticipate the timing of his or her ingestion of a meal such that the delayed-release formulation can be administered in time for it to take effect before the meal is begun, or even that a meal will cause symptoms necessitating treatment with a proton pump inhibitor. As such, it is desirable to have a proton pump inhibitor formulation that can be administered to a fed patient (e.g., with food, shortly after initiating ingestion of food, or at any time within the period of time after initiating ingestion of food where symptoms requiring administration of the formulation arise) in an immediate-release formulation such that the patient is treated in a timely manner after initiating ingestion of a meal.

### SUMMARY OF THE INVENTION

The present invention provides a pharmaceutical composition comprising a proton pump inhibiting agent and a buffering agent for oral administration and ingestion by a subject. In one embodiment, upon administration to a fed subject, the composition contacts the gastric fluid of the stomach and increases the gastric pH of the stomach to a pH that prevents or inhibits acid degradation of the proton pump inhibiting agent in the gastric fluid of the stomach and allows a measurable serum concentration of the proton pump inhibiting agent to be absorbed into the blood serum of the subject, such that pharmacokinetic and pharmacodynamic parameters can be obtained using testing procedures known to those skilled in the art.

Pharmaceutical compositions including (a) a therapeutically effective amount of at least one acid labile proton pump inhibitor, and (b) at least one buffering agent in an amount sufficient to increase gastric fluid pH to a pH that prevents acid degradation of at least some of the proton pump inhibitor in the gastric fluid. Methods are provided for treating gastric acid related disorders using pharmaceutical composition of the present invention.

Proton pump inhibitors include, but are not limited to, omeprazole, hydroxyomeprazole, esomeprazole, tenatoprazole, lansoprazole, pantoprazole, rabeprazole, dontoprazole, habeprazole, periprazole, ransoprazole, pariprazole, leminoprazole; or a free base, free acid, salt, hydrate, ester, amide, enantiomer, isomer, tautomer, polymorph, or prodrug thereof. In one embodiment, the proton pump inhibitor is omeprazole or a free base, free acid, salt, hydrate, ester, amide, enantiomer, isomer, tautomer, polymorph, or prodrug thereof. Compositions can contain between about 5 mgs to about 500 mgs of proton pump inhibitor, specifically about 10 mg, about 15 mg, about 20 mg, about 30 mg, about 40 mgs, or about 60 mgs of the proton pump inhibitor.

Compositions are provided wherein the proton pump inhibitor is microencapsulated with a material that enhances the shelf-life of the pharmaceutical composition. The material that enhances the shelf-life of the pharmaceutical composition includes, but is not limited to, cellulose hydroxypropyl ethers, low-substituted hydroxypropyl ethers, cellulose hydroxypropyl methyl ethers, methylcellulose polymers, ethylcelluloses and mixtures thereof, polyvinyl alcohol, hydroxyethylcelluloses, carboxymethylcelluloses, salts of carboxymethylcelluloses, polyvinyl alcohol, polyethylene glycol co-polymers, monoglycerides, triglycerides, polyethylene glycols, modified food starch, acrylic polymers, mixtures of acrylic polymers with cellulose ethers, cellulose acetate phthalate, sepifilms, cyclodextrins; and mixtures thereof. The cellulose hydroxypropyl ether can be, but is not limited to, Klucel®, Nisswo HPC or PrimaFlo HP22. The cellulose hydroxypropyl methyl ether can be, but is not limited to, Seppifilm-LC, Pharmacoat®, Metolose SR, Opadry YS, PrimaFlo, MP3295A, BenecelMP824, or BenecelMP843. The mixture of methylcellulose and hydroxypropyl and methylcellulose polymers can be, but is not limited to, Methocel®, Benecel-MC, or Metolose®. The ethylcellulose or mixture thereof can be, but is not limited to, Ethocel®, BenecelMO43, Celacal, Cumibak NC, and E461. The polyvinyl alcohol can be, but is not limited to, Opadry AMB. Composition can include a mixture wherein the hydroxyethylcellulose is Natrosol®, the carboxymethylcellulose is Aqualon®-CMC, the polyvinyl alcohol and polyethylene glycol co-polymer is Kollicoat IR®, and the acrylic polymers are selected from Eudragits® EPO, Eudragits® RD100, and Eudragits(g) E100. The material that enhances the shelf-life of the pharmaceutical composition can further include an antioxidant, a plasticizer, a buffering agent, or mixtures thereof.

Compositions are provided that include (a) a therapeutically effective amount of at least one acid labile proton pump inhibitor, wherein at least some of the proton pump inhibitor is coated, and (b) at least one buffering agent in an amount sufficient to increase gastric fluid pH to a pH that prevents acid degradation of at least some of the proton pump inhibitor in the gastric fluid.

Compositions including (a) a therapeutically effective amount of at least one acid labile proton pump inhibitor, and (b) at least one buffering agent in an amount sufficient to increase gastric fluid pH to a pH that prevents acid degradation of at least some of the proton pump inhibitor in the gastric fluid are provided, wherein the buffering agent is an alkaline metal salt or a Group IA metal selected from a bicarbonate salt of a Group IA metal, a carbonate salt of a Group IA metal. The buffering agent can be, but is not limited to, an amino acid, an acid salt of an amino acid, an alkali salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, and mixtures thereof. In particular, the buffering agent can be sodium bicarbonate, sodium carbonate, calcium carbonate, magnesium oxide, magnesium hydroxide, magnesium carbonate, aluminum hydroxide, and mixtures thereof.

Compositions are provided as described herein, wherein the buffering agent is sodium bicarbonate present in about 0.1 mEq/mg proton pump inhibitor to about 5 mEq/mg proton pump inhibitor. Compositions are provided as described herein, wherein the buffering agent is a mixture of sodium bicarbonate and magnesium hydroxide, and each buffering agent is present in about 0.1 mEq/mg proton pump inhibitor to about 5 mEq/mg proton pump inhibitor. Compositions are provided as described herein, wherein the buffering agent is a mixture of sodium bicarbonate, calcium carbonate, and magnesium hydroxide, and each buffering agent is present in about 0.1 mEq/mg proton pump inhibitor to about 5 mEq/mg of the proton pump inhibitor.

Compositions are provided as described herein, wherein the buffering agent is present in an amount of about 0.1 mEq/mg to about 5 mEq/mg of the proton pump inhibitor, or about 0.5 mEq/mg to about 3 mEq/mg of the proton pump inhibitor, or about 0.8 mEq/mg to about 2.5 mEq/mg of the proton pump inhibitor, or about 0.9 mEq/mg to about 2.0 mEq/mg of the proton pump inhibitor, or about 0.9 mEq/mg to about 1.8 mEq/mg of the proton pump inhibitor. Compositions are provided as described herein, wherein the buffering agent is present in an amount of at least 1.0 mEq/mg to about 1.5 mEq/mg of the proton pump inhibitor, or at least about 0.4 mEq/mg of the proton pump inhibitor. Compositions are provided as described herein, including about 200 to 3000 mg of buffering agent, or about 500 to about 2500 mg of buffering agent, or about 1000 to about 2000 mg of buffering agent, or about 1500 to about 2000 mg of buffering agent.

Compositions are provided such that when administered to a subject prior to a meal, the gastric pH is maintained above about 4.0 for at least about 1 hour following the meal. Compositions are provided such that when administered to a subject prior to a meal, the gastric pH is maintained above about 4.2 for at least about 1 hour following the meal. Compositions are provided such that when administered to a subject prior to a meal, the gastric pH is maintained above about 4.5 for at least about 1 hour following the meal.

Compositions are provided such that when administered to a subject prior to a meal, the gastric pH of the subject is increased to at least about 3 within about 1 hour after administration. Compositions are provided such that when administered to a subject prior to a meal, the gastric pH of the subject is increased to at least about 3 within about 45 minutes after administration. Compositions are provided such that when administered to a subject prior to a meal, the gastric pH of the subject is increased to at least about 3 within about 30 minutes after administration. Compositions are provided such that when administered to a subject prior to a meal, the gastric pH of the subject is increased to at least about 3 within about 15 minutes after administration.

Compositions are provided such that when administered to a subject prior to a meal, the gastric pH of the subject is increased to at least about 4 within about 1 hour after administration. Compositions are provided such that when administered to a subject prior to a meal, the gastric pH of the subject is increased to at least about 4 within about 45 minutes after administration. Compositions are provided such that when administered to a subject prior to a meal, the gastric pH of the subject is increased to at least about 4 within about 30 minutes after administration. Compositions are provided such that when administered to a subject prior to a meal, the gastric pH of the subject is increased to at least about 4 within about 15 minutes after administration.

Compositions are provided wherein a therapeutically effective amount of the proton pump inhibitor is absorbed within about 1 hour after administration. Compositions are provided wherein a therapeutically effective amount of the proton pump inhibitor is absorbed within 45 minutes after administration. Compositions are provided wherein a therapeutically effective amount of the proton pump inhibitor is absorbed within about 30 minutes after administration.

Compositions are provided such that the maximum gastric pH is reached within about 45 minutes after administration of the composition. Compositions are provided such that the maximum gastric pH is reached within about 30 minutes after administration of the composition. Compositions are provided such that the maximum gastric pH is reached within about 15 minutes after administration of the composition. Compositions are provided such that the maximum gastric pH is reached within about 10 minutes after administration of the composition.

Compositions are provided such that the gastric pH is greater then about 4.0 at least about 50% of the time. Compositions are provided such that the gastric pH is greater then about 4.0 at least about 60% of the time. Compositions are provided such that the gastric pH is greater then about 4.0 at least about 70% of the time. Compositions are provided such that the gastric pH is greater then about 4.0 at least about 80% of the time. Compositions are provided wherein, upon oral administration to the subject, the composition provides a pharmacokinetic profile such that at least about 50% of total area under serum concentration time curve (AUC) for the proton pump inhibitor occurs within about 2 hours after administration of a single dose of the composition to the subject. Compositions are provided wherein, upon oral administration to the subject, the area under the serum concentration time curve (AUC) for the proton pump inhibitor in the first 2 hours is at least about 60% of the total area. Compositions are provided wherein the area under the serum concentration time curve (AUC) for the proton pump inhibitor in the first 2 hours is at least about 70% of the total area.

Compositions are provided wherein at least about 50% of total area under the serum concentration time curve (AUC) for the proton pump inhibitor occurs within about 1.75 hours after administration of a single dose of the composition to the subject. Compositions are provided wherein the at least about 50% of total area under the serum concentration time curve (AUC) for the proton pump inhibitor occurs within about 1.5 hours after administration of a single dose of the composition to the subject. Compositions are provided wherein the at least about 50% of total area under the serum concentration time curve (AUC) for the proton pump inhibitor occurs within about 1 hour after administration of a single dose of the composition to the subject.

Compositions including (a) a therapeutically effective amount of at least one acid labile proton pump inhibitor, and (b) at least one buffering agent in an amount sufficient to increase gastric fluid pH to a pH that prevents acid degradation of at least some of the proton pump inhibitor in the gastric fluid, wherein the composition is in a dosage form selected from a powder, a tablet, a bite-disintegration tablet, a chewable tablet, a capsule, an effervescent powder, a rapid-disintegration tablet, or an aqueous suspension produced from powder. Compositions are provided as described herein, further including one or more excipients including, but not limited to, parietal cell activators, erosion facilitators, flavoring agents, sweetening agents, diffusion facilitators, antioxidants and carrier materials selected from binders, suspending agents, disintegration agents, filling agents, surfactants, solubilizers, stabilizers, lubricants, wetting agents, diluents, anti-adherents, and antifoaming agents. Compositions are also provided wherein at least some of the proton pump inhibitor is micronized.

Compositions comprising (a) an amount of at least one acid labile proton pump inhibitor; and (b) at least one buffering agent in an amount sufficient to inhibit or reduce degradation of at least some of the proton pump inhibitor are provided such that when the composition is administered to a subject before a meal the composition causes a increase in gastric pH to above 3.0 within 30 minutes after administration. Compositions comprising (a) an amount of at least one acid labile proton pump inhibitor; and (b) at least one buffering agent in an amount sufficient to inhibit or reduce degradation of at least some of the proton pump inhibitor are provided such that when the composition is administered to a subject before a meal the composition causes a increase in gastric pH to about 3.0 within about 1 hour after administration.

Compositions are provided comprising (a) a therapeutically effective amount of at least one acid labile proton pump inhibitor; and (b) at least one buffering agent in an amount sufficient to inhibit or reduce degradation of at least some of the proton pump inhibitor by gastric fluid, wherein the composition is in an amount effective to reduce or inhibit upper GI bleeding following administration to the subject. Compositions are provided wherein the composition is administered in a liquid formulation and reduces mortality or nosocomial pneumonia due to upper GI bleeding, or a complication associated with upper GI bleeding.

Compositions are provided comprising (a) a therapeutically effective amount of at least one acid labile proton pump inhibitor; and (b) at least one buffering agent in an amount sufficient to inhibit or reduce degradation of at least some of the proton pump inhibitor by gastric fluid are provided for the treatment of gastric acid related disorders. Gastric acid related disorders include, but are not limited to, duodenal ulcer disease, gastric ulcer disease, gastroesophageal reflux disease, erosive esophagitis, poorly responsive symptomatic gastroesophageal reflux disease, pathological gastrointestinal hypersecretory disease, Zollinger Ellison syndrome, heartburn, esophageal disorder, or acid dyspepsia.

Methods are provided for preventing or inhibiting breakthrough of pH control in a subject by administering a compund comprising (a) a therapeutically effective amount of at least one acid labile proton pump inhibitor; and (b) at least one buffering agent in an amount sufficient to inhibit or reduce degradation of at least some of the proton pump inhibitor by gastric fluid, wherein the subject has previously been administered a compound within about the past 2-22 hours that increases gastric pH to about 3, thereby preventing or inhibiting breakthrough of pH control. Methods are provided such that the composition useful for preventing or inhibiting breakthrough of pH control is administered before retiring to bed. Methods are provided such that the composition useful for preventing or inhibiting breakthrough of pH control is administered to treat or prevent nocturnal heartburn. Methods are provided such that integrated gastric acidity in the subject is reduced by at least about 25% to about 500%.

Methods for rapidly reducing production of gastric acid in a subject by administering a composition comprising (a) a therapeutically effective amount of at least one acid labile proton pump inhibitor; and (b) at least one buffering agent in an amount sufficient to inhibit or reduce degradation of at least some of the proton pump inhibitor by gastric fluid are provided herein. Also provided herein are methods of treating a gastric acid related disorder induced by a meal by administering a composition comprising (a) a therapeutically effective amount of at least one acid labile proton pump inhibitor; and (b) at least one buffering agent in an amount sufficient to inhibit or reduce degradation of at least some of the proton pump inhibitor by gastric fluid.

Methods for treating a gastric acid related disorder induced by a meal in a subject by administering to the subject within about 4 hours following ingestion of the meal a composition comprising, (a) at least one acid labile proton pump inhibitor; and (b) at least one buffering agent in an amount sufficient to inhibit or reduce degradation of at least some of the proton pump inhibitor are provided herein such that the amount of proton pump inhibitor is effective to reduce or inhibit one or more symptoms of the gastric acid related disorder in the subject.

Methods of treating a critically ill subject having or at risk of having upper GI bleeding or a symptom associated with upper GI bleeding comprising administering to the subject a liquid formulation comprising at least one acid labile proton pump inhibitor, and at least one buffering agent in an amount sufficient to inhibit or reduce degradation of at least some of the proton pump inhibitor are provided such that the amount of proton pump inhibitor is effective to reduce or inhibit upper GI bleeding or the symptom associated with upper GI bleeding in the critically ill subject. Methods of treating a critically ill subject having or at risk of having upper GI bleeding or a sysmpton associated with upper GI bleeding are provided such that the subject has a nasogastric (NG) tube or a gastric tube. Methods are also provided herein for reducing the incidence, severity, duration or frequency of upper GI bleeding or one or more symptoms associated with upper GI bleeding in the subject. Methods are provided herein for reducing mortality or nosocomial pneumonia associated with upper GI bleeding in the subject.

Methods of treating a patient having a gastric acid related disorder or at risk of having a gastric acid related disorder, wherein the subject has difficulty swallowing a pill, capsule, caplet or tablet, by administering to the subject a liquid formulation comprising at least one acid labile proton pump inhibitor and at least one buffering agent in an amount sufficient to inhibit or reduce degradation of at least some of the proton pump inhibitor.

Methods for treating a patient suffering from heartburn or at risk of suffering from heartburn by administering a pharmaceutical composition comprising (a) a therapeutically effective amount of at least one acid labile proton pump inhibitor; and (b) at least one buffering agent in an amount sufficient to inhibit or reduce degradation of at least some of the proton pump inhibitor by gastric fluid, are also provided herein.

### BRIEF DESCRIPTION OF THE DRAWINGS

Other advantages of the present invention will be readily appreciated as the same becomes better understood by reference to the following detailed description when considered in connection with the accompanying drawing wherein:

FIG. 1 is a line graph illustrating the mean plasma omeprazole concentrations measured over the time period of six (6) hours after administration of 40 mg omeprazole/antacid immediate-release formulation (OAC-IR) and 40 mg omeprazole delayed-release formulation (OME-DR) to fasting subjects.

FIG. 2 is a line graph illustrating the Day 1 mean plasma omeprazole concentrations for 40 mg omeprazole plus sodium bicarbonate administered after an overnight fast and for 40 mg Prilosec® administered after an overnight fast.

FIG. 3 is a line graph illustrating the Day 7 mean plasma omeprazole concentrations for 40 mg omeprazole plus sodium bicarbonate administered after an overnight fast and for 40 mg Prilosec® administered after an overnight fast.

FIG. 4(a) illustrates the integrated gastric acidity at baseline (untreated) and Days 1 and 7 of 40 mg omeprazole plus sodium bicarbonate administered after an overnight fast.

FIG. 4(b) illustrates the integrated gastric acidity at baseline (untreated) and Days 1 and 7 of 40 mg Prilosec® administered after an overnight fast.

FIG. 5(a) illustrates the phasic changes in gastric acid concentration produced by the ingestion of meals with administration of 40 mg omeprazole plus sodium bicarbonate after an overnight fast at Days 1 and 7; baseline (untreated) values are also presented.

FIG. 5(b) illustrates the phasic changes in gastric acid concentration produced by the ingestion of meals with administration of 40 mg Prilosec® after an overnight fast at Days 1 and 7; baseline (untreated) values are also presented.

FIG. 6(a) illustrates the median gastric pH measured on Day 1 after administration of 40 mg omeprazole plus sodium bicarbonate after an overnight fast and the median gastric pH measured after administration of 40 mg Prilosec® after an overnight fast.

FIG. 6(b) illustrates the median gastric pH measured on Day 7 after administration of 40 mg omeprazole plus sodium bicarbonate after an overnight fast and the median gastric pH measured after administration of 40 mg Prilosec® after an overnight fast.

FIG. 7(a) illustrates Day 1 values showing the time gastric pH was <4 with administration of 40 mg omeprazole plus sodium bicarbonate after an overnight fast and the time gastric pH was ≦4 with administration of 40 mg Prilosec® after an overnight fast.

FIG. 7(b) illustrates Day 7 values showing the time gastric pH was <4 with administration of 40 mg omeprazole plus sodium bicarbonate after an overnight fast and the time gastric pH was ≦4 with administration of 40 mg Prilosec® administered after an overnight fast.

FIGS. 8(a) and 8(b) are line graphs summarizing the mean ratios and confidence intervals for pharmacokinetic and pharmacodynamic parameters after 7 days of daily administration of omeprazole plus sodium bicarbonate, and Prilosec®. FIG. 8(a) shows parameters calculated after 7 days of daily administration of 20 mg omeprazole plus sodium bicarbonate after an overnight fast and 20 mg Prilosec®, each of which was administered after an overnight fast. FIG. 8(b) presents parameters calculated after 7 days of daily administration of 40 mg omeprazole plus sodium bicarbonate and 40 mg Prilosec®, each of which was administered after an overnight fast.

FIG. 9 is a line graph illustrating the mean plasma omeprazole concentrations on Day 7 for 40 mg omeprazole plus sodium bicarbonate administered pre-meal and after an overnight fast; and illustrating the mean plasma omeprazole concentration on Day 8 for 40 mg omeprazole plus sodium bicarbonate administered post-meal.

FIG. 10 is a line graph illustrating the mean plasma omeprazole concentrations from fasting subjects following administration of: 40 mg omeprazole plus antacid in the SAN-05 powder formulation; 40 mg omeprazole plus antacid in the SAN-15 chewable tablet formulation; and 40 mg Prilosec® in a delayed-release (enteric-coated) formulation.

FIG. 11 is a line graph illustrating: the bioavailability of 40 mg of omeprazole plus sodium bicarbonate in the SAN-15 chewable tablet formulation administered 30 minutes premeal; and the bioavailability of 40 mg of Nexium® administered 30 minutes premeal.

FIG. 12 is a bar graph illustrating the cumulative integrated gastric acidity after administration of different omeprazole formulations: Rapinex® chewable tablet formulation; Acitrel® suspension formulation; and Prilosec® delayed-release formulation.

FIG. 13 is a line graph illustrating the effect on gastric pH of administering: 40 mg omeprazole as the SAN-15 formulation (40 mg omeprazole plus sodium bicarbonate) administered either 30 or 60 minutes pre-meal; Nexium® 30 minutes pre-meal; Prilosec® 30 minutes premeal; and gastric pH of untreated subjects.

FIG. 14 is a bar graph illustrating the effect on postmeal integrated gastric acidity of administering: 40 mg omeprazole plus sodium bicarbonate in the SAN-15 formulation either 30 or 60 minutes pre-meal; Nexium®; and no omeprazole (control).

FIG. 15(a) is a line graph illustrating the mean gastric acid pH over time following administration of 40 mg omeprazole plus sodium bicarbonate in the SAN-15 formulation; control values represent the gastric acid pH of untreated subjects.

FIG. 15(b) is a line graph illustrating the mean gastric acid pH over time following administration of 80 mg omeprazole plus sodium bicarbonate in the SAN-15 formulation; control values represent the gastric acid pH of untreated subjects.

FIG. 15(c) is a line graph illustrating the mean gastric acid pH over time following administration of 120 mg omeprazole plus sodium bicarbonate in the SAN-15 formualtion; control values represent the gastric acid pH of untreated subjects.

FIG. 16 is a line graph illustrating the plasma omeprazole concentration following administration of 40 mg omeprazole plus sodium bicarbonate in the SAN-15 formulation, comparing results from administration to fed subjects, administration 1 hour post-meal.

FIG. 17 is a line graph illustrating the mean plasma omeprazole concentration following two doses of 40 mg omeprazole in the OSB-IR formulation, administered six hours apart.

FIG. 18(a) is a line graph illustrating the median gastric pH for 24 hours following administration of 40 mg omeprazole plus sodium bicarbonate in the OSB-IR formulation on Day 1 of treatment of qAM treatment.

FIG. 18(b) is a line graph illustrating the median gastric pH for 24 hours following administration of 40 mg omeprazole plus sodium bicarbonate in the OSB-IR formulation on Day 7 of qAM treatment.

FIGS. 19(a) and 19(b) are bar graph illustrations of the integrated gastric acidity of subjects treated with 20 mg omeprazole plus sodium bicarbonate in the OSB-IR formulation on Day 1 and Day 7. FIG. 19(a) presents the the daytime gastric acidity. FIG. 19(b) presents the nocturnal gastric acidity. In each figure, results for untreated subjects are presented as baseline values.

FIGS. 20(a) and 20(b) are bar graph illustrations of the integrated gastric acidity of subjects treated daily with 40 mg omeprazole plus sodium bicarbonate in the OSB-IR formulation on Day 1 and Day 7. FIG. 20(a) presents the daytime gastric acidity. FIG. 20(b) presents the nocturnal gastic acidity. In each figure, results for untreated subjects are presented as baseline values.

FIGS. 21(a) and 21(b) are line graphs illustrating the Day 7 median gastric acid pH over time following administration of 20 mg omeprazole plus sodium bicarbonate in the OSB-IR formulation (FIG. 21(a)) or 40 mg omeprazole plus sodium bicarbonate in the OSB-IR formulation (FIG. 21(b)); results for untreated subjects are presented as baseline values.

FIG. 22 is a bar graph illustrating the postprandial integrated gastric acidity following each of three daily meals, on Day 1 and Day 7 of daily (qAM) administration of 20 mg omeprazole plus sodium bicarbonate in the OSB-IR formulation; results for untreated subjects are presented as baseline values.

FIG. 23 is a bar graph illustrating the postprandial integrated gastric acidity following each of three daily meals, on Day 1 and Day 7 of daily (qAM) administration of 40 mg omeprazole plus sodium bicarbonate in the OSB-IR formulation; results for untreated subjects are presented as baseline values.

FIGS. 24(a) to 24(c) are line drawings illustrating the median gastric pH over 24 hours on Day 7 of daily (qAM) administration of 40 mg omeprazole plus sodium bicarbonate in the OSB-IR formulation (FIG. 24(a)); the median gastric pH over 24 hours on Day 7 of daily (qAM) administration of 20 mg omeprazole plus sodium bicarbonate in the OSB-IR formulation (FIG. 24(b)); and the median gastric pH over 24 hours on Day 8 wherein a second dose of 20 mg omeprazole plus sodium bicarbonate in the OSB-IR formulation (FIG. 24(c)) was administered at bedtime.

FIG. 25 is a bar graph illustrating the number of critically ill patients in a cimetidine-treated population and the number of critically ill patients in an omeprazole-treated (OSB-IR) population having the following: a pH value lower than 4 in two successive aspirates; any evidence of bleeding; and clinically significant bleeding.

FIG. 26 is a line graph illustrating the pre-dose and post-dose gastric pHs in critically ill patients dosed during the first 2 days of treatment with three doses of a suspension of 40 mg omeprazole (OSB-IR formulation) or with 1200 mg/day intravenous (IV) cimetidine.

FIG. 27 is a line graph illustrating the median gastric pH over 14 days in critically ill patients dosed either with a suspension of 40 mg/day of omeprazole (OSB-IR formulation) or with 1200 mg/day intravenous (IV) cimetidine.

FIG. 28 is a non-inferiority analysis for the difference in bleeding rates which illustrates the difference between the OSB-IR bleeding rate and the cimetidine bleeding rate.

### DETAILED DESCRIPTION OF THE INVENTION

The present invention is directed to methods, kits, combinations, and compositions for treating a condition or disorder where treatment with an H+, K+-ATPase inhibiting agent or inhibitor, such as, for example, a proton pump inhibiting agent, is indicated. Also provided are methods, kits, combinations, and compositions for treating, preventing or reducing the risk of developing a gastrointestinal disorder or disease, or the symptoms associated with, or related to a gastrointestinal disorder or disease in a subject in need thereof.

While the present invention may be embodied in many different forms, several specific embodiments are discussed herein with the understanding that the present disclosure is to be considered only as an exemplification of the principles of the invention, and it is not intended to limit the invention to the embodiments illustrated. For example, where the present invention is illustrated herein with particular reference to omeprazole, hydroxyomeprazole, esomeprazole, tenatoprazole, lansoprazole, pantoprazole, rabeprazole, dontoprazole, habeprazole, periprazole, ransoprazole, pariprazole, or leminoprazole, it will be understood that any other proton pump inhibiting agent, if desired, can be substituted in whole or in part for such agents in the methods, kits, combinations, and compositions herein described.

Glossary

To more readily facilitate an understanding of the invention and its preferred embodiments, the meanings of terms used herein will become apparent from the context of this specification in view of common usage of various terms and the explicit definitions of other terms provided in the glossary below or in the ensuing description.

As used herein, the terms “comprising,” “including,” and “such as” are used in their open, non-limiting sense.

The use of the term “about” in the present disclosure means “approximately,” and illustratively, the use of the term “about” indicates that values slightly outside the cited values may also be effective and safe, and such dosages are also encompassed by the scope of the present claims.

As used herein, the phrase “acid-labile pharmaceutical agent” refers to any pharmacologically active drug subject to acid catalyzed degradation.

“Anti-adherents,” “glidants,” or “anti-adhesion” agents prevent components of the formulation from aggregating or sticking and improve flow characteristics of a material. Such compounds include, e.g., colloidal silicon dioxide such as Cab-o-sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®)and the like.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, e.g., butylated hydroxytoluene (BHT), sodium ascorbate, and tocopherol.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

“Bioavailability” refers to the extent to which an active moiety (drug or metabolite) is absorbed into the general circulation and becomes available at the site of drug action in the body.

The term “bioequivalence” or “bioequivalent” means that two drug products do not differ significantly when the two products are administered at the same dose under similar conditions. A product can be considered bioequivalent to a second product if there is no significant difference in the rate and extent to which the active ingredient or active moiety becomes available at the site of drug action when the product is administered at the same molar dose as the second product under similar conditions in an appropriately designed study. Two products with different rates of absorption can be considered equivalent if the difference in the rate at which the active ingredient or moiety becomes available at the site of drug action is intentional and is reflected in the proposed labeling, is not essential to the attainment of effective body drug concentrations on chronic use, and is considered medically insignificant for the drug. Bioequivalence can be assumed when, for example, the 90% confidence interval ranges between 80% and 120% for the target parameters (e.g., Cₘₐₓ and AUC).

“Carrier materials” include any commonly used excipients in pharmaceutics and should be selected on the basis of compatibility with the proton pump inhibitor and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

The term “controlled release” includes any nonimmediate release formulation, including but not limited to enteric-coated formulations and sustained release, delayed-release and pulsatile release formulations.

The term “delayed-release” includes any nonimmediate release formulation, including but not limited to, film-coated formulations, enteric-coated formulations, encapsulated formulations, sustained release formulations and pulsatile release formulations. See Remington: The Science and Practice of Pharmacy, (20^{th} Ed. 2000). As discussed herein, immediate and nonimmediate release (or controlled release) can be defined kinetically by reference to the following equation:

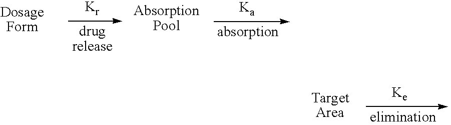

The absorption pool represents a solution of the drug administered at a particular absorption site, and Kᵣ, Kₐ, and Kₑ are first-order rate constants for: (1) release of the drug from the formulation; (2) absorption; and (3) elimination, respectively. For immediate release dosage forms, the rate constant for drug release Kᵣ, is generally equal to or greater than the absorption rate constant Kₐ. For controlled release formulations, the opposite is generally true, that is, Kᵣ, <<Kₐ, such that the rate of release of drug from the dosage form is the rate-limiting step in the delivery of the drug to the target area.

“Diffusion facilitators” and “dispersing agents” include materials that control the diffusion of an aqueous fluid through a coating. Exemplary diffusion facilitators/dispersing agents include, e.g., hydrophilic polymers, electrolytes, Tween® 60 or 80, PEG and the like. Combinations of one or more erosion facilitator with one or more diffusion facilitator can also be used in the present invention.

“Diluents” increase bulk of the composition to facilitate compression. Such compounds include e.g., lactose; starch; mannitol; sorbitol; dextrose; microcrystalline cellulose such as Avicel®; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); mannitol; hydroxypropylmethylsellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; mannitol; sodium chloride; inositol; bentonite; and the like.

The term “disintegrate” includes both the dissolution and dispersion of the dosage form when contacted with gastric fluid. “Disintegration agents” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

“Drug absorption” or “absorption” refers to the process of movement from the site of administration of a drug toward the systemic circulation.

“Drug elimination” or “elimination” refers to the sum of the processes of drug loss from the body.

“Erosion facilitators” include materials that control the erosion of a particular material in gastroic fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose; dextrates; dextran, starches, pregelatinized starch, sucrose, xylitol, lactitol, mannitol, sorbitol, sodium chloride, polyethylene glycol, and the like.

“Flavoring agents” or “sweeteners” useful in the pharmaceutical compositions of the present invention include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

The terms “therepeutically effective amount” and “effective amount” in relation to the amount of proton pump inhibiting agent mean, consistent with considerations known in the art, the amount of proton pump inhibiting agent effective to elicit a pharmacologic effect or therapeutic effect (including, but not limited to, raising of gastric pH, raising pH in esophagus, reducing gastrointestinal bleeding, reducing in the need for blood transfusion, improving survival rate, more rapid recovery, H+, K+-ATPase inhibition or improvement or elimination of symptoms, and other indicators as are selected as appropriate measures by those skilled in the art), without undue adverse side effects. “Effective amount” in the context of a buffering agent means an amount sufficient to prevent the acid degradation of the PPI, in whole or in part, either in vivo or in vitro.

An “enteric-coating” is a substance that remains substantially intact in the stomach but dissolves and releases at least some of the drug once reaching the small intesting. Generally, the enteric-coating comprises a polymeric material that prevents release in the low pH environment of the stomach but that ionizes at a slightly higher pH, typically a pH of 4 or 5, and thus dissolves sufficiently in the small intestines to gradually release the active agent therein.

“Fasting adult human subject” or “fasting subject” refers to, for example, any patient who has abstained from food for a period of time, e.g., a patient who has not ingested a meal overnight (e.g., 8 hours), a patient who has not ingested a meal in several hours, a patient with an empty stomach who is not suffering any meal-related symptoms that can be treated with a proton pump inhibitor, or any patient who has not ingested a meal such that the most recently ingested meal is digested and the patient is not suffering from any meal-related symptoms that can be treated with a proton pump inhibitor.

“Fed adult human subject” or “fed subject” refers to, for example, a patient who is initiating ingestion of a meal, a patient who has initiated ingestion of a meal a short time before administration (e.g., at about 10 minutes before, at about 20 minutes before, at about 30 minutes before, at about 45 minutes before, at about 60 minutes before, or at about 90 minutes before), a patient who has initiated ingestion of a meal a short time before administration and continues to ingest food after administration, a patient who has recently finished ingesting a meal, or a patient who has finished ingesting a meal and who is experiencing symptoms related to the ingestion of that meal.

The phrase “gastrointestinal disorder” or “gastrointestinal disease” refers generally to a disorder or disease that occurs in a mammal due to an imbalance between acid and pepsin production, called aggressive factors, and mucous, bicarbonate, and prostaglandin production, called defensive factors. In mammals, such disorders or diseases include, but are not limited to, duodenal ulcer, gastric ulcer, acid dyspepsia, gastroesophageal reflux disease (GERD), severe erosive esophagitis, poorly responsive symptomatic gastroesophageal reflux disease, heartburn, other esophageal disorders, irritable bowel syndrome, and a gastrointestinal pathological hypersecretory condition such as Zollinger Ellison Syndrome. Treatment of these conditions is accomplished by administering to a subject a therapeutically effective amount of a pharmaceutical composition according to the present invention.

The phrase “gastrointestinal fluid” or “gastric fluid” refers to the fluid of stomach secretions of a subject or the equivalent thereof. An equivalent of stomach secretion includes, for example, an in vitro fluid having a similar content and/or pH as the stomach secretions. The content and pH of a particular stomach secretion is generally subject specific, and depends upon, among other things, the weight, sex, age, diet, or health of a particular subject. These particular stomach secretions can, for example, be mimicked or replicated by those skilled in the art, for example, those found in in vitro models used to study the stomach. One such model is commonly known as the “Kinetic Acid Neutralization Model,” and can be used to experimentally study or determine release kinetics (for example, immediate release versus control release) of a component of the compositions of the present invention under predetermined experimental conditions; or acid degradation of a pharmaceutical agent of the compositions herein described under predetermined experimental conditions.

“Half-life” refers to the time required for the plasma drug concentration or the amount in the body to decrease by 50% from its maximum concentration.

The use of the term “highly acidic pH” in the present disclosure means a pH in the range of about 1 to about 4.

The term “immediate release” is intended to refer to any PPI formulation in which all or part of the PPI is in solution either before administration or immediately (i.e., within about 30 minutes) after administration. For example, with an “immediate release” formulation, oral administration results in immediate release of the agent from the composition into gastric fluid. For delayed-release formulations, the opposite is generally true, the rate of release of drug from the dosage form is the rate-limiting step in the delivery of the drug to the target area.

“Integrated acidity” is calculated as the cumulative time-weighted average mean gastric acid concentration. Integrated gastric acidity is expressed in mmol×hr/L and is calculated from gastric pH data obtained (about every 8 seconds) using a pH probe (electrode). Put another way, integrated gastric acidity can be calculated from time-weighted average hydrogen ion concentrations over a 24-hour recording period.

The “Kinetic Acid Neutralization Model” is an in vitro model used to study the subject. Briefly, in the Kinetic Acid Neutralization Model, the timed acid neutralization of an amount of buffering agent or agents, for example, a representative amount of calcium carbonate, and/or sodium bicarbonate can be evaluated. While not intending to be bound by any one theory, it is generally believed that a healthy human stomach adds HCl to the stomach contents at the rate of 30 mL per hour. The Kinetic Acid Neutralization Model uses a glass flask (in the form of a 100 mL or 200 mL dissolution flask, for example) to hold 0.1 N hydrochloric acid (HCl) (to simulate the acidity of the stomach in the fasted state). Fifty mL is considered the volume of acid usually found in a fasted stomach, but for experimental convenience, the model can, for example, utilized 100 mL (double the usual fasted stomach volume). An overhead stirrer maintains at a constant, controlled and reproducible rpm, stirring the contents in the flask. For the analysis of pH, an Orion pH Meter (model 720A) equipped with an Orion pH electrode (combination probe/PerpHeot Ross Semimicro Electrode) can be employed, for example. The Kinetic Acid Neutralization Model can add, by a peristaltic pump (Watson/Marlow Multichannel PumpPro model with acid resistant tubing), 200 mL per hour of 0.05 N HCl. This rate compensates for the doubling of the initial volume of 0.1 N HCl from 50 to 100 mL. To simulate stomach emptying, fluid can be withdrawn from the flask at the same rate and by the same peristaltic pump, maintaining the 100 mL volume constant. This Kinetic Acid Neutralization Model combines the concepts of USP<301>, Acid-Neutralizing Capacity Test, and the concepts of USP<724>, the Flow Through Cell for Drug Release Testing, which are incorporated herein by reference. Illustratively, the pH of the initial acid in the flask can be measured as a function of time. At time zero, the buffering agent is added to the flask, and the pH of the contents measured, starting at one minute intervals, and progressing at convenient time intervals until the pH falls below a predetermined level, for example, a value of 3 or less. When testing a controlled-release dosage form of the present invention in this model, the amount of the agent released from the dosage form into the gastric fluid and/or the acid-degradation of the agent can be determined by, for example, High Performance Liquid Chromatography (HPLC).

The use of the term “less acidic to basic pH” means a pH between about 4 to about 8.0.

“Lubricants” are compounds which prevent, reduce or inhibit adhesion or friction of materials. Exemplary lubricants include, e.g., stearic acid; calcium hydroxide; talc; sodium stearyl fumerate; a hydrocarbon such as mineral oil, or hydrogenated vegetable oil such as hydrogenated soybean oil (Sterotex®); higher fatty acids and their alkali-metal and alkaline earth metal salts, such as aluminum, calcium, magnesium, zinc, stearic acid, sodium stearates, glycerol, talc, waxes, Stearowet® boric acid, sodium benzoate, sodium acetate, sodium chloride, leucine, a polyethylene glycol or a methoxypolyethylene glycol such as Carbowax™, sodium oleate, glyceryl behenate, polyethylene glycol, magnesium or sodium lauryl sulfate, colloidal silica such as Syloid™, Carb-O-Sil®, a starch such as corn starch, silicone oil, a surfactant, and the like.

“Meal” refers to, for example, any amount of food, e.g., a snack, a serving of food, several servings of one food, one or several servings each of different foods, or any amount of food that induces symptoms necessitating treatment with a proton pump inhibitor.

The term “measurable serum concentration” means the serum concentration (typically measured in mg, μg, or ng of therapeutic agent per ml, dl, or l of blood serum) of a therapeutic agent absorbed into the bloodstream after administration. Illustratively, the serum concentration of a proton pump inhibiting agent of the present invention that corresponds to a measurable serum concentration for an adult subject is greater than about 5 ng/ml. In another embodiment of the present invention, the serum concentration of the proton pump inhibiting agent that corresponds to a measurable serum concentration for an adult human is less than about 10 ng/ml. In yet another embodiment of the present invention, the serum concentration of the proton pump inhibiting agent that corresponds to a measurable serum concentration for an adult human is from about 10 ng/ml to about 500 ng/ml. And in still another embodiment of the present invention, the serum concentration of the proton pump inhibiting agent that corresponds to a measurable serum concentration for an adult human is from about 250 ng/ml to about 2500 ng/ml.

“Metabolism” refers to the process of chemical alteration of drugs in the body.

“Parietal cell activators” or “activators” stimulate the parietal cells and enhance the pharmaceutical activity of the proton pump inhibitor. Parietal cell activators include, e.g., chocolate; alkaline substances such as sodium bicarbonate; calcium such as calcium carbonate, calcium gluconate, calcium hydroxide, calcium acetate and calcium glycerophosphate; peppermint oil; spearmint oil; coffee; tea and colas (even if decaffeinated); caffeine; theophylline; theobromine; amino acids (particularly aromatic amino acids such as phenylalanine and tryptophan); and combinations thereof.

The term “pharmaceutically acceptable” is used adjectivally herein to mean that the modified noun is appropriate for use in a pharmaceutical product.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

The term “pharmacologically active drug” and its equivalents, includes at least one of any therapeutically, prophylactically and/or pharmacologically or physiologically beneficial active substance, or mixture thereof, which is delivered to a living subject to produce a desired, usually therapeutic, effect. More specifically, any drug which is capable of producing a pharmacological response, localized or systemic, irrespective of whether therapeutic, diagnostic, or prophylactic in nature, particularly in mammals, is within the contemplation of the invention.

“Plasma concentration” refers to the concentration of a substance in blood plasma or blood serum of a subject. It is understood that the plasma concentration of a therapeutic agent may vary many-fold between subjects, due to variability with respect to metabolism of therapeutic agents. In accordance with one aspect of the present invention, the plasma concentration of a proton pump inhibitors and/or nonsteroidal anti-inflammatory drug may vary from subject to subject. Likewise, values such as maximum plasma concentraton (Cₘₐₓ) or time to reach maximum serum concentration (Tₘₐₓ), or area under the serum concentration time curve (AUC) may vary from subject to subject. Due to this variability, the amount necessary to constitute “a therapeutically effective amount” of proton pump inhibitor, nonsteroidal anti-inflammatory drug, or other therapeutic agent, may vary from subject to subject. It is understood that when mean plasma concentrations are disclosed for a population of subjects, these mean values may include substantial variation.

The term “prevent” or “prevention,” in relation to a gastrointestinal disorder or disease, means no gastrointestinal disorder or disease development if none had occurred, or no further gastrointestinal disorder or disease development if there had already been development of the gastrointestinal disorder or disease. Also considered is the ability of one to prevent some or all of the symptoms associated with the gastrointestinal disorder or disease.

“Solubilizers” include compounds such as citric acid, succinic acid, fumaric acid, malic acid, tartaric acid, maleic acid, glutaric acid, sodium bicarbonate, sodium carbonate and the like.

“Stabilizers” include compounds such as any antioxidation agents, buffers, acids, and the like.

“Suspending agents” or “thickening agents” include compounds such as polyvinylpyrrolidone, e.g., polyvinylpyrrolidone K12, polyvinylpyrrolidone K17, polyvinylpyrrolidone K25, or polyvinylpyrrolidone K30; polyethylene glycol, e.g., the polyethylene glycol can have a molecular weight of about 300 to about 6000, or about 3350 to about 4000, or about 7000 to about 5400; sodium carboxymethylcellulose; methylcellulose; hydroxy-propylmethylcellulose; polysorbate-80; hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone and the like.

“Surfactants” include compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF); and the like.

As used herein, the terms “suspension” and “solution” are interchangeable with each other and generally mean a solution and/or suspension of the substituted benzimidazole in an aqueous medium.

The term “sustained release” is used in its conventional sense to refer to a drug formulation that provides for gradual release of a drug over an extended period of time, and, may sometimes, although not necessarily, result in substantially constant blood levels of a drug over an extended time period.

“Therapeutic window” refers to the range of plasma concentrations, or the range of levels of therapeutically active substance at the site of action, with a high probability of eliciting a therapeutic effect.

The term “treat” or “treatment” as used herein refers to any treatment of a disorder or disease associated with gastrointestinal disorder, and includes, but is not limited to, preventing the disorder or disease from occurring in a mammal which may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder.

Proton Pump Inhibitors

For the purposes of this application, the term “proton pump inhibitor,” or “PPI,” or “proton pump inhibiting agent” means any agent possessing pharmacological activity as an inhibitor of H⁺, K⁺-ATPase. The definition of “PPI,” or “proton pump inhibitor,” or “proton pump inhibiting agent” as used herein can also mean that the agent possessing pharmacological activity as an inhibitor of H⁺,K⁺-ATPase can, if desired, encompass all related chemical forms, which may be in the form of a free base, free acid, a salt, an ester, a hydrate, an amide, an enantiomer, an isomer, a tautomer, a polymorph, a prodrug, a derivative or the like, provided such forms are suitable pharmacologically, that is, effective in the present methods, combinations, kits, and compositions. After oral administration to the subject and absorption of the proton pump inhibiting agent (or administration intravenously), the agent is delivered via the serum to various tissues and cells of the body including the parietal cells. Not intending to be bound by any one theory, research suggests that when the proton pump inhibiting agent is in the form of a weak base and is non-ionized, it freely passes through physiologic membranes, including the cellular membranes of the parietal cell. It is believed that the non-ionized proton pump inhibiting agent moves into the acid-secreting portion of the parietal cell, the secretory canaliculus. Once in the acidic milieu of the secretory canaliculus, the proton pump inhibiting agent is apparently protonated (ionized) and converted to the active form of the drug. Generally, ionized proton pump inhibiting agents are membrane impermeable and form disulfide covalent bonds with cysteine residues in the alpha subunit of the proton pump. Such active forms are included within the definition of “PPI,” “proton pump inhibitor,” or “proton pump inhibiting agent” as used herein.

A class of proton pump inhibiting agents useful in the methods, kits, combinations, and compositions of the present invention are substituted benzimidazole (including, for example, substituted benzimidazoles wherein the benzimidazole ring itself is substituted with a nitrogen to form a 6-membered pyridine ring attached to the imidazole ring). In one embodiment, the substituted benzimidazole is of the formula (I):

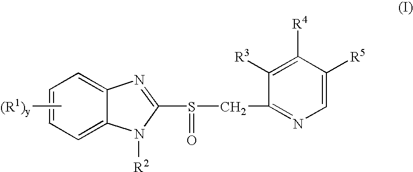

wherein R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl;

R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl;

R³ and R⁵ are the same or different and each is hydrogen, alkyl, alkoxy or alkoxyalkoxy;

R⁴ is hydrogen, alkyl, alkoxy which may optionally be fluorinated, or alkoxyalkoxy; and

y is an integer of 0 through 4;

or a free base, free acid, salt, hydrate, ester, amide, enantiomer, isomer, tautomer, polymorph, or prodrug thereof.

Illustratively, a substituted benzimidazole of interest that can be used in the methods, kits, combinations, and compositions of the present invention includes, but is not limited to, omeprazole, hydroxyomeprazole, lansoprazole, pantoprazole, rabeprazole, dontoprazole, esomeprazole (also known as s-omeprazole or perprazole), tenatoprazole, habeprazole, ransoprazole, pariprazole, and leminoprazole; or a free base, free acid, salt, hydrate, ester, amide, enantiomer, isomer, tautomer, polymorph, prodrug, or derivative of these compounds. (Based in part upon the list provided in The Merck Index, Merck & Co. Rahway, N.J. (2001)).

Examples of salt forms of proton pump inhibiting agents include, for example, a sodium salt form, such as, esomeprazole sodium, omeprazole sodium, rabeprazole sodium, pantoprazole sodium; or a magnesium salt form, such as, esomeprazole magnesium or omeprazole magnesium as described in U.S. Pat. No. 5,900,424; or a calcium salt form; or a potassium salt form, such as, the potassium salt of esomeprazole as described in U.S. patent application No. 2002/0198239, and U.S. Pat. No. 6,511,996. Other salts of esomeprazole are described in U.S. Pat. No. 4,738,974 and U.S. Pat. No. 6,369,085, for example.

Included in the methods, kits, combinations and pharmaceutical compositions of the present invention are the isomeric forms and tautomers of the described compounds and the pharmaceutically acceptable salts thereof. Examples of substituted benzimidazole tautomers useful in the present invention, include tautomers of omeprazole, as described in U.S. Pat. Nos. 6,262,085; 6,262,086; 6,268,385; 6,312,723; 6,316,020; 6,326,384; 6,369,087; and 6,444,689; and U.S. patent application Publication No. 02/0156103, all by Whittle, et al.

Examples of isomers of substituted benzimidazoles useful in the present invention include an isomer of omeprazole. For example, the compound 5-methoxy-2-[[(4-methoxy-3,5-dimethyl-2-pyridinyl)methyl]sulfinyl]-1H-benzimidazole, having the generic name omeprazole, as well as therapeutically acceptable salts thereof, are described in EP 5129. The single crystal X-ray data and the derived molecular structure of a crystalline form of omeprazole are described by Oishi et al., Acta Cryst. (1989), C45, 1921-1923. This crystal form of omeprazole has been referred to as omeprazole form B. Another crystalline form of omeprazole referred to as omeprazole form A is described in U.S. Pat. No. 6,150,380, and U.S. patent application Publication No. 02/0156284, by Lovqvist et al. Still yet another crystalline form of omeprazole is described in WO 02/085889, by Hafnler et al.

Examples of suitable polymorphs are described in, for example, U.S. Pat. Nos. 4,045,563; 4,182,766; 4,508,905; 4,628,098; 4,636,499; 4,689,333; 4,758,579; 4,783,974; 4,786,505; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; and 6,444,689.

Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Pharmaceutically acceptable cations include metallic ions and organic ions. Illustratively, metallic ions include, but are not limited to appropriate alkali metal (Group IA) salts, alkaline earth metal (Group IIA) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the methods, kits, combinations and pharmaceutical compositions of the present invention are the prodrugs of the described compounds and the pharmaceutically acceptable salts thereof. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug, which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak et al., Am. J. Physiol, 269:G210-218 (1995), describe dexamethasone-beta-D-glucuronide. McLoed et al., Gastroenterol., 106:405-413 (1994), describe dexamethasone-succinate-dextrans. Hochhaus et al., Biomed. Chrom., 6:283-286 (1992), describe dexamethasone-21-sulphobenzoate sodium and dexamethasone-21 -isonicotinate. Additionally, J. Larsen and H. Bundgaard [Int. J. Pharmaceutics, 37, 87 (1987)] describe the evaluation of N-acylsulfonamides as potential prodrug derivatives. J. Larsen et al., [Int. J. Pharmaceutics, 47, 103 (1988)] also describe the evaluation of N-methylsulfonamides as potential prodrug derivatives. Prodrugs are also described in, for example, Sinkula et al., J. Pharm. Sci., 64:181-210 (1975).

Other substituted benzimidazole compounds and the salts, hydrates, esters, amides, enantiomers, isomers, tautomers, polymorphs, prodrugs and derivatives thereof may be prepared using standard procedures known to those skilled in the art of synthetic organic chemistry and described, for example, by J. March, Advanced Organic Chemistry; Reactions, Mechanisms and Structure, 4th Ed. (New York: Wiley-Interscience, 1992).

Combinations and mixtures of the above-mentioned proton pump inhibiting agent can be used in the methods, kits, combinations, and compositions herein described. Salts, hydrates, esters, amides, enantiomers, isomers, tautomers, polymorphs, prodrugs, and derivatives of the proton pump inhibiting agent may be prepared using standard procedures known to those skilled in the art of synthetic organic chemistry and described, for example, in J. March, Advanced Organic Chemistry; Reactions, Mechanisms and Structure, 4th Ed. (New York: Wiley-Interscience, 1992). For example, acid addition salts are prepared from the free base using conventional methodology, and involve reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, for example, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, for example, hydrochloric acid, hydrobromic acid, sulfuric acid, nitric acid, phosphoric acid, and the like. An acid addition salt may be reconverted to the free base by treatment with a suitable base. In one embodiment, the acid addition salts of the active agents herein are halide salts, such as may be prepared using hydrochloric or hydrobromic acids. In yet another embodiment, the basic salts here are alkali metal salts, for example, the sodium salt, and copper salts.

Preparation of esters involves functionalization of hydroxyl and/or carboxyl groups which may be present within the molecular structure of the drug. The esters are typically acyl-substituted derivatives of free alcohol groups, that is, moieties that are derived from carboxylic acids of the formula RCOOH where the H is replaced with a lower alkyl group. Esters can be reconverted to the free acids, if desired, by using conventional hydrogenolysis or hydrolysis procedures. Amides may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

As utilized herein, the term “acyl,” alone or in combination, means a radical provided by the residue after removal of hydroxyl from an organic acid. Examples of such acyl radicals include alkanoyl and aroyl radicals. Examples of such alkanoyl radicals include formyl, acetyl, propionyl, butyryl, isobutyryl, valeryl, isovaleryl, pivaloyl, hexanoyl, trifluoroacetyl, and the like.

The term “alkoxy” or “alkyloxy,” alone or in combination, mean an alkyl ether radical wherein the term alkyl is as defined above. Examples of suitable alkyl ether radicals include methoxy, ethoxy, n-propoxy, isopropoxy, n-butoxy, iso-butoxy, sec-butoxy, tert-butoxy, and the like. The “alkoxy” radicals may be further substituted with one or more halo atoms, such as fluoro, chloro or bromo, to provide haloalkoxy radicals. Illustratively, haloalkoxy radicals are “haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxyalkyl,” alone or in combination, means an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals.

The term “alkyl,” alone or in combination, means a straight-chain or branched-chain alkyl radical containing one to about twelve carbon atoms, preferably one to about ten carbon atoms, and more preferably one to about six carbon atoms. Examples of such radicals include methyl, ethyl, n-propyl, isopropyl, n-butyl, isobutyl, sec-butyl, tert-butyl, pentyl, iso-amyl, hexyl, octyl, and the like.

The term “alkylsulfinyl,” alone or in combination, means a radical containing a linear or branched alkyl radical, of one to ten carbon atoms, attached to a divalent —S(═O)— radical. Illustratively, alkylsulfinyl radicals are radicals having alkyl radicals of one to six carbon atoms. Examples of such alkylsulfmyl radicals include methylsulfinyl, ethylsulfinyl, butylsulfmyl and hexylsulfinyl.

The term “alkylsulfonyl,” alone or in combination, means an alkyl radical attached to a sulfonyl radical, where alkyl is defined as above. Illustratively, alkylsulfonyl radicals are alkylsulfonyl radicals having one to six carbon atoms. Examples of such alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The term “alkylthio,” alone or in combination, means a radical containing a linear or branched alkyl radical, of one to about ten carbon atoms attached to a divalent sulfur atom. Illustratively, alkylthio radicals are radicals having alkyl radicals of one to six carbon atoms. Examples of such alkylthio radicals are methylthio, ethylthio, propylthio, butylthio and hexylthio.

The term “alkylthioalkyl,” alone or in combination, means a radical containing an alkylthio radical attached through the divalent sulfur atom to an alkyl radical of one to about ten carbon atoms. Illustratively, alkylthioalkyl radicals are radicals having alkyl radicals of one to six carbon atoms. Examples of such alkylthioalkyl radicals include methylthiomethyl, methylthioethyl, ethylthioethyl, and ethylthiomethyl.

The term “amino,” alone or in combination, means an amine or —NH₂ group whereas the term mono-substituted amino, alone or in combination, means a substituted amine —N(H)(substituent) group wherein one hydrogen atom is replaced with a substituent, and disubstituted amine means a —N(substituent)₂ wherein two hydrogen atoms of the amino group are replaced with independently selected substituent groups.

Amines, amino groups and amides are compounds that can be designated as primary (I°), secondary (II°) or tertiary (III°) or unsubstituted, mono-substituted or N,N-disubstituted depending on the degree of substitution of the amino nitrogen. Quaternary amine (ammonium)(IV°) means a nitrogen with four substituents [—N⁺(substituent)₄] that is positively charged and accompanied by a counter ion, whereas N-oxide means one substituent is oxygen and the group is represented as [—N⁺(substituent)₃-O⁻]; that is, the charges are internally compensated.

The term “aminoalkyl,” alone or in combination, means an alkyl radical substituted with amino radicals. Preferred are aminoalkyl radicals having alkyl portions having one to six carbon atoms. Examples of such radicals include aminomethyl, aminoethyl, and the like.

The termd “arylalkyl” or “aralkyl” alone or in combination, means an alkyl radical as defined above in which one hydrogen atom is replaced by an aryl radical as defined above, such as benzyl, diphenylmethyl, triphenylmethyl, phenylethyl, diphenylethyl 2-phenylethyl, and the like. The aryl in said aralkyl may be additionally substituted with halo, alkyl, alkoxy, halkoalkyl and haloalkoxy. The terms benzyl and phenylmethyl are interchangeable.

The term “aryl,” alone or in combination, means a five- or six-membered carbocyclic aromatic ring-containing moiety or a five- or six-membered carbocyclic aromatic system containing two or three rings wherein such rings are attached together in a pendent manner, or a fused ring system containing two or three rings that have all carbon atoms in the ring; that is, a carbocyclic aryl radical. The term “aryl” embraces aromatic radicals such as phenyl, indenyl, naphthyl, tetrahydronaphthyl, indane and biphenyl. Aryl moieties may also be substituted with one or more substituents including alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The termd “carbonyl” or “oxo,” alone or in combination, that is, used with other terms, such as “alkoxycarbonyl,” means a —C(═O)— group wherein the remaining two bonds (valences) can be independently substituted. The term carbonyl is also intended to encompass a hydrated carbonyl group —C(OH)2—.

The terms “carboxy” or “carboxyl,” whether used alone or in combination, that is, with other terms, such as “carboxyalkyl,” mean a —CO₂H radical.

The term “carboxyalkyl,” alone or in combination, means an alkyl radical substituted with a carboxy radical. Illustratively, carboxyalkyl radicals have alkyl radicals as defined above, and may be additionally substituted on the alkyl radical with halo. Examples of such carboxyalkyl radicals include carboxymethyl, carboxyethyl, carboxypropyl, and the like.

The term “cyano,” alone or in combination, means a —C-triple bond-N (—C≡N) group.

The term “cycloalkyl,” alone or in combination, means a cyclic alkyl radical that contains three to about twelve carbon atoms. Illustratively, cycloalkyl radicals are cycloalkyl radicals having three to about eight carbon atoms. Examples of such radicals include cyclopropyl, cyclobutyl, cyclopentyl, cyclohexyl, and the like.

The term “derivative” refers to a compound that is produced from another compound of similar structure by the replacement of substitution of one atom, molecule or group by another. For example, a hydrogen atom of a compound may be substituted by alkyl, acyl, amino, hydroxyl, halo, haloalkyl, etc., to produce a derivative of that compound.

The term “halo” or “halogen,” alone or in combination, means halogen such as fluoride, chloride, bromide or iodide.

The term “haloalkyl”, alone or in combination, means an alkyl radical having the significance as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. In some embodiments, the haloalkyl radicals are haloalkoxy radicals having one to six carbon atoms and one or more halo radicals. Examples of such haloalkyl radicals include chloromethyl, dichloromethyl, trichloromethyl, 1-bromoethyl, fluoromethyl, difluoromethyl, trifluoromethyl, 1,1,1-trifluoroethyl, pentafluoroethyl, heptafluoropropyl, difluorochloromethyl, dichlorofluoromethyl, difluoroethyl, difluoropropyl, dichloroethyl, dichloropropyl, and the like.

The term “heteroaryl,” alone or in combination means a five- or six-membered aromatic ring-containing moiety or a fused ring system (radical) containing two or three rings that have carbon atoms and also one or more heteroatoms in the ring(s) such as sulfur, oxygen and nitrogen. Examples of such heterocyclic or heteroaryl groups are pyrrolidinyl, piperidyl, piperazinyl, morpholinyl, thiamorpholinyl, pyrrolyl, imidazolyl (for example, imidazol-4-yl, 1 -benzyloxycarbonylimidazol-4-yl, and the like), pyrazolyl, pyridyl, pyrazinyl, pyrimidinyl, furyl, tetrahydrofuryl, thienyl, triazolyl, tetrazolyl, oxazolyl, oxadiazoyl, thiazolyl, thiadiazoyl, indolyl (for example, 2-indolyl, and the like), quinolinyl, (for example, 2-quinolinyl, 3-quinolinyl, 1-oxido-2-quinolinyl, and the like), isoquinolinyl (for example, 1-isoquinolinyl, 3-isoquinolinyl, and the like), tetrahydroquinolinyl (for example, 1,2,3,4-tetrahydro-2-quinolyl, and the like), 1,2,3,4-tetrahydroisoquinolinyl (for example, 1,2,3,4-tetrahydro-1-oxo-isoquinolinyl, and the like), quinoxalinyl, β-carbolinyl, 2-benzofurancarbonyl, benzothiophenyl, 1-, 2-, 4- or 5-benzimidazolyl, and the like radicals.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated three- to six-membered heteromonocylic group containing one to four nitrogen atoms (for example pyrrolidinyl, imidazolidinyl, piperidino, piperazinyl, etc.); saturated three- to six-membered heteromonocyclic group containing one to two oxygen atoms and one to three nitrogen atoms (for example morpholinyl, etc.); saturated three- to six-membered heteromonocyclic group containing one to two sulfur atoms and one to three nitrogen atoms (for example, thiazolidinyl, etc.). Examples of partially unsaturated heterocyclo radicals include dihydrothiophene, dihydropyran, dihydrofuran and dihydrothiazole. A heterocyclic (heterocyclo) portion of a heterocyclocarbonyl, heterocyclooxy-carbonyl, heterocycloalkoxycarbonyl, or heterocycloalkyl group or the like is a saturated or partially unsaturated monocyclic, bicyclic or tricyclic heterocycle that contains one or more hetero atoms selected from nitrogen, oxygen and sulphur. Heterocyclo compounds include benzofused heterocyclic compounds such as benzo-1,4-dioxane. Such a moiety can be optionally substituted on one or more ring carbon atoms by halogen, hydroxy, hydroxycarbonyl, alkyl, alkoxy, oxo, and the like, and/or on a secondary nitrogen atom (that is, —NH—) of the ring by alkyl, aralkoxycarbonyl, alkanoyl, aryl or arylalkyl or on a tertiary nitrogen atom (that is, ═N—) by oxido and that is attached via a carbon atom. The tertiary nitrogen atom with three substituents can also attached to form a N-oxide [═N(O)—] group.

The term “heterocycloalkyl,” alone or in combination, means a saturated and partially unsaturated heterocyclo-substituted alkyl radical, such as pyrrolidinylmethyl, and heteroaryl-substituted alkyl, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The terms “hydrido” or “hydrogen,” alone or in combination, means a single hydrogen atom (H). This hydrido radical may be attached, for example, to an oxygen atom to form a hydroxyl radical or two hydrido radicals may be attached to a carbon atom to form a methylene (—CH₂—) radical.

The term “hydroxyalkyl,” alone or in combination, means a linear or branched alkyl radical having one to about ten carbon atoms any one of which may be substituted with one or more hydroxyl radicals. Preferred hydroxyalkyl radicals have one to six carbon atoms and one or more hydroxyl radicals. Examples of such radicals include hydroxymethyl, hydroxyethyl, hydroxypropyl, hydroxybutyl and hydroxyhexyl.

The term “hydroxyl,” alone or in combination, means a —OH group.

The term “nitro,” alone or in combination, means a —NO₂ group.

The term “prodrug” refers a drug or compound in which the pharmacological action results from conversion by metabolic processes within the body. Prodrugs are generally drug precursors that, following administration to a subject and subsequent absorption, are converted to an active, or a more active species via some process, such as conversion by a metabolic pathway. Some prodrugs have a chemical group present on the prodrug which renders it less active and/or confers solubility or some other property to the drug. Once the chemical group has been cleaved and/or modified from the prodrug the active drug is generated. Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak, et al., Am. J. Physio.l, 269:G210-218 (1995); McLoed, et al., Gastroenterol., 106:405-413 (1994); Hochhaus, et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987.

The term “sulfone,” alone or in combination, means a —SO₂— group wherein the depicted remaining two bonds (valences) can be independently substituted.

The term “sulfonyl,” alone or in combination, that is, linked to other terms such as alkylsulfonyl, means a —SO₂— group wherein the depicted remaining two bonds (valences) can be independently substituted.

The term “sulfoxido,” alone or in combination, means a —SO— group wherein the remaining two bonds (valences) can be independently substituted.

The term “thiol” or “sulfhydryl,” alone or in combination, means a —SH group. The term “thio” or “thia,” alone or in combination, means a thiaether group; that is, an ether group wherein the ether oxygen is replaced by a sulfur atom.

Buffering Agents

The terms “buffering agent” or “buffer” mean any pharmaceutically appropriate weak base or strong base (and mixtures thereof) which, when formulated or delivered before, during and/or after the proton pump inhibiting agent, functions to substantially prevent or inhibit the acid degradation of the proton pump inhibiting agent by gastric acid sufficient to preserve the bioavailability of the proton pump inhibiting agent administered.

The pharmaceutical compositions of the invention comprises one or more buffering agents. A class of buffering agents useful in the present invention include, but are not limited to, buffering agents possessing pharmacological activity as a weak base or a strong base. In one embodiment, the buffering agent, when formulated or delivered with an proton pump inhibiting agent, functions to substantially prevent or inhibit the acid degradation of the proton pump inhibitor by gastric fluid for a period of time, e.g., for a period of time sufficient to preserve the bioavailability of the proton pump inhibitor administered. The buffering agent can be delivered before, during and/or after delivery of the proton pump inhibitor. In one aspect of the present invention, the buffering agent includes a salt of a Group IA metal (alkali metal), including, e.g., a bicarbonate salt of a Group IA metal, a carbonate salt of a Group IA metal; an alkaline earth metal buffering agent (Group IIA metal); an aluminum buffering agent; a calcium buffering agent; or a magnesium buffering agent.

Other buffering agents suitable for the present invention include, e.g., alkali metal (a Group IA metal including, but not limited to, lithium, sodium, potassium, rubidium, cesium, and francium) or alkaline earth metal (Group IIA metal including, but not limited to, beryllium, magnesium, calcium, strontium, barium, radium) carbonates, phosphates, bicarbonates, citrates, borates, acetates, phthalates, tartrate, succinates and the like, such as sodium or potassium phosphate, citrate, borate, acetate, bicarbonate and carbonate.

In various embodiments, a buffering agent includes an amino acid, an alkali metal salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol. (See, e.g., lists provided in The Merck Index, Merck & Co. Rahway, N.J. (2001)). Certain proteins or protein hydrolysates that rapidly neutralize acids can serve as buffering agents in the present invention. Combinations of the above mentioned buffering agents can be used in the pharmaceutical compositions described herein.

The buffering agents useful in the present invention also include buffering agents or combinations of buffering agents that interact with HCl (or other acids in the environment of interest) faster than the proton pump inhibitor interacts with the same acids. When placed in a liquid phase, such as water, these buffering agents produce and maintain a pH greater than the pKa of the proton pump inhibitor.

In various embodiments, the buffering agent is selected from sodium bicarbonate, sodium carbonate, calcium carbonate, magnesium oxide, magnesium hydroxide, magnesium carbonate, aluminum hydroxide, and mixtures thereof. In another embodiment, the buffering agent is sodium bicarbonate and is present in about 0.1 mEq/mg proton pump inhibitor to about 5 mEq/mg proton pump inhibitor. In yet another embodiment, the buffering agent is a mixture of sodium bicarbonate and magnesium hydroxide, wherein the sodium bicarbonate and magnesium hydroxide are each present in about 0.1 mEq/mg proton pump inhibitor to about 5 mEq/mg proton pump inhibitor. In still another embodiment, the buffering agent is a mixture of at least two buffers selected from sodium bicarbonate, calcium carbonate, and magnesium hydroxide, wherein each buffer is present in about 0.1 mEq/mg proton pump inhibitor to about 5 mEq/mg of the proton pump inhibitor.

Compositions are provided as described herein, wherein the buffering agent is present in an amount of about 0.1 mEq/mg to about 5 mEq/mg of the proton pump inhibitor, or about 0.25 mEq/mg to about 3 mEq/mg of the proton pump inhibitor, or about 0.3 mEq/mg to about 2.5 mEq/mg of the proton pump inhibitor, or about 0.4 mEq/mg to about 2.0 mEq/mg of the proton pump inhibitor, or about 0.5 mEq/mg to about 1.5 mEq/mg of the proton pump inhibitor. Compositions are provided as described herein, wherein the buffering agent is present in an amount of at least 0.25 mEq/mg to about 2.5 mEq/mg of the proton pump inhibitor, or at least about 0.4 mEq/mg of the proton pump inhibitor.

In one aspect of the invention, compositions are provided wherein the buffering agent is present in the pharmaceutical compositions of the present invention in an amount of about 1 mEq to about 160 mEq per dose, or about 5 mEq, or about 10 mEq, or about 11 mEq, or about 12 mEq, or about 13 mEq, or about 15 mEq, or about 19 mEq, or about 20 mEq, or about 21 mEq, or about 22 mEq, or about 23 mEq, or about 24 mEq, or about 25 mEq, or about 30 mEq, or about 31 mEq, or about 35 mEq, or about 40 mEq, or about 45 mEq, or about 50 mEq, or about 60 mEq, or about 70 mEq, or about 80 mEq, or about 90 mEq, or about 100 mEq, or about 110 mEq, or about 120 mEq, or about 130 mEq, or about 140 mEq, or about 150 mEq, or about 160 mEq per dose.

In another aspect of the invention, compositions are provided wherein the buffering agent is present in the composition in an amount, on a weight to weight (w/w) basis, of more than about 5 times, or more than about 10 times, or more than about 20 times, or more than about 30 times, or more than about 40 times, or more than about 50 times, or more than about 60 times, or more than about 70 times, or more than about 80 times, or more than about 90 times, or more than about 100 times the amount of the proton pump inhibiting agent.

In another aspect of the invention, compositions are provided wherein the amount of buffering agent present in the pharmaceutical composition is between 200 and 3500 mg. In some embodiments, the amount of buffering agent present in the pharmaceutical composition is about 200 mg, or about 300 mg, or about 400 mg, or about 500 mg, or about 600 mg, or about 700 mg, or about 800 mg, or about 900 mg, or about 1000 mg, or about 1100 mg, or about 1200 mg, or about 1300 mg, or about 1400 mg, or about 1500 mg, or about 1600 mg, or about 1700 mg, or about 1800 mg, or about 1900 mg, or about 2000 mg, or about 2100 mg, or about 2200 mg, or about 2300 mg, or about 2400 mg, or about 2500 mg, or about 2600 mg, or about 2700 mg, or about 2800 mg, or about 2900 mg, or about 3000 mg, or about 3200 mg, or about 3500 mg;

Combination Therapy

The phrase “combination therapy” means the administration of a composition of the present invention in conjunction with another pharmaceutical agent. The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Substantially simultaneous administration can be accomplished, for example, by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval.

The therapeutic compounds of the combined therapy whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally (for example, sublingual), or parenterally (for example, subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

Combination therapy includes, for example, administration of a composition of the present invention in conjunction with another pharmaceutical agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected).

For example, the present methods, kits, and compositions can be used in combination with another pharmaceutical agent that is indicated for treating or preventing a gastrointestinal disorder, such as, for example, an anti-bacterial agent, an alginate, a prokinetic agent, a H₂-antagonist, an antacid, or sucralfate, which are commonly administered to minimize the pain and/or complications related to this disorder. These drugs have certain disadvantages associated with their use. Some of these drugs are not completely effective in the treatment of the aforementioned conditions and/or produce adverse side effects, such as mental confusion, constipation, diarrhea, and thrombocytopenia. H₂-antagonists, such as ranitidine and cimetidine, are relatively costly modes of therapy, particularly in NPO patients, which frequently require the use of automated infusion pumps for continuous intravenous infusion of the drug. However, when used in conjunction with the present invention, that is, in combination therapy, many if not all of these unwanted side effects can be reduced or eliminated. The reduced side effect profile of these drugs is generally attributed to, for example, the reduce dosage necessary to achieve a therapeutic effect with the administered combination.

In another example, the present methods, kits, and compositions can be used in combination with other pharmaceutical agents, including but not limited to: NSAIDs including but not limited to aminoarylcarboxylic acid derivatives such as enfenamic acid, etofenamate, flufenamic acid, isonixin, meclofenamic acid, mefenamic acid, niflumic acid, talniflumate, terofenamate, and tolfenamic acid; arylacetic acid derivatives such as aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac isoxepac, lonazolac, metiazinic acid, mofezolac, oxametacine, pirazolac, proglumetacin, sulindac, tiaramide, tolmetin, tropesin, and zomepirac; arylbutyric acid derivatives such as bumadizon, butibufen, fenbufen, xenbucin; arylcarboxylic acids such as clidanac, ketorolac, tinoridine; arylpropionic acid derivatives such as alminoprofen, benoxaprofin, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprofin, pirprofen, pranoprofen, protizinic acid, suprofen, tiaprofenic acid, ximoprofen, and zaltoprofen; pyrazoles such as difenamizole, and epirozole; pyrazolones such as apazone, benzpiperylon, feprazone, mofebutazone, morazone, oxyphenbutazone, phenylbutazone, pipebuzone, propyphenazone, prostaglandins, ramifenazone, suxibuzone, and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphtyl salicylate, olsalazine, parsalmide, phenyl acetylsalicylate, phenyl salicylate, salacetamide, salicylamide o-acetic acid, salicylsulfuric acid, salsalate, sulfasalazine; thiazinecarboxamides such as ampiroxicam, droxicamn, isoxicam, lomoxicam, piroxicam, and tenoxicam; cyclooxygenase-II inhibitors (“COX-II”) such as Celebrex (Celecoxib), Vioxx, Relafen, Lodine, and Voltaren and others, such as epsilon-acetamidocaproic acid, s-adenosylmethionine, 3-amino-4-hydroxybutytic acid, amixetrine, bendazac, benzydamine, α-bisabolol, bucololome, difenpiramide, ditazol, emorfazone, fepradinol, guaiazulene, nabumetone, nimesulide, oxaceprol, paranyline, perisoxal, proquazone, tenidap and zilenton; sleep aids including but not limited to a benzodiazepine hypnotic, non-benzodiazepine hypnotic, antihistamine hypnotic, antidepressant hypnotic, herbal extract, barbiturate, peptide hypnotic, triazolam, brotizolam, loprazolam, lormetazepam, flunitrazepam, flurazepam, nitrazepam, quazepam, estazolam, temazepam, lorazepam, oxazepam, diazepam, halazepam, prazepam, alprazolam, chlordiazepoxide, clorazepate, an imidazopyridine or pyrazolopyrimidine hypnotic, zolpidem or zolpidem tartarate, zopiclone, eszopiclone, zaleplon, indiplone, diphenhydramine, doxylamine, phenyltoloxamine, pyrilamine, doxepin, amtriptyline, trimipramine, trazodon, nefazodone, buproprion, bupramityiptyline, an herbal extract such as valerian extract or amentoflavone, a hormone such as melatonin,or gabapeptin; motility agents, including but not limited to 5-HT inhibitors such as cisapride, domperidone, and metoclopramide, and agents useful for treating irritable bowel syndrome.

Compositions

The present invention provides pharmaceutical compositions comprising a proton pump inhibiting agent and a buffering agent for oral administration and ingestion by a subject. The composition can comprise any suitable proton pump inhibiting agent, e.g., omeprazole, hydroxyomeprazole, esomeprazole, lansoprazole, pantoprazole, rabeprazole, dontoprazole, esomeprazole (also known as s-omeprazole or perprazole), habeprazole, perprazole, ransoprazole, pariprazole, and leminoprazole; or a free base, free acid, a salt, hydrate, ester, amide, enantiomer, isomer, tautomer, polymorph, prodrug, or derivative of these compounds. The composition can comprise any suitable buffering agent, that, when formulated or delivered before, during and/or after the proton pump inhibiting agent, functions to substantially prevent or inhibit the acid degradation of the proton pump inhibiting agent by gastric acid sufficient to preserve the bioavailability of the proton pump inhibiting agent administered, such as, for example, sodium salts, potassium salts, magnesium salts, calcium salts, aluminum hydroxide, aluminum hydroxide/sodium bicarbonate coprecipitate, a mixture of an amino acid and a buffer, a mixture of aluminum glycinate and a buffer, a mixture of an acid salt of an amino acid and a buffer, and a mixture of an alkali salt of an amino acid and a buffer, or any other suitable buffering agent or mixture of buffering agents. In one embodiment, the present invention relates to a pharmaceutical composition comprising a proton pump inhibiting agent, a buffering agent, and optionally a parietal cell activator.

The therapeutic agents of the present invention can be formulated as a single pharmaceutical composition or as independent multiple pharmaceutical dosage forms. Pharmaceutical compositions according to the present invention include those suitable for oral, rectal, buccal (for example, sublingual), or parenteral (for example, intravenous) administration, although the most suitable route in any given case will depend on the nature and severity of the condition being treated and on the nature of the particular compound which is being used. The therapeutic agents can be formulated in any suitable dosage forms, such as, e.g., tablets including chewable tablets, caplets, powders, suspensions, capsules, or any other suitable dosage form known in the art.

In another embodiment of the present invention, the composition of the present invention comes in the form of a kit or package containing one or more of the compositions or therapeutic agents of the present invention. The composition containing the composition or therapeutic agent can be packaged in the form of a kit or package in which hourly, daily, weekly, or monthly (or other periodic) dosages are arranged for proper sequential or simultaneous administration. The present invention further provides a kit or package containing a plurality of dosage units, adapted for successive daily administration, each dosage unit comprising at least one of the compositions or therapeutic agents of the present invention. This drug delivery system can be used to facilitate administration of any of the various embodiments of the compositions and therapeutic agents of the present invention. In one embodiment, the system contains a plurality of doses to be to be administered daily or as needed for symptomatic relief. The kit or package can also contain agents utilized in combination therapy to facilitate proper administration of the dosage forms. The kit or package can also contain a set of instructions for the subject.

The pharmaceutical composition of the present invention can be prepared in any suitable dosage form. Suitable dosage forms include, but are not limited to, a tablet, a caplet, a powder, a suspension tablet, a chewable tablet, a capsule, an effervescent powder, an effervescent tablet, a seed, a pellet, a bead, a microcapsule, a mini-tablet, a spheroid, a microsphere, an agglomerate, a granule, or any other multi-particulate forms manufactured by conventional pharmacological techniques.

In one embodiment of the present invention, the compositions comprise a dry formulation, or a solution and/or a suspension of the proton pump inhibiting agent. Such dry formulations, solutions and/or suspensions may also include, for example, a suspending agent (for example, gums, xanthans, cellulosics and sugars), a humectant (for example, sorbitol), a solubilizer (for example, ethanol, water, PEG and propylene glycol), a surfactant (for example, sodium lauryl sulfate, Spans, Tweens, and cetyl pyridine), a preservative, an antioxidant (for example, parabens, and vitamins E and C), an anti-caking agent, a coating agent, a chelating agent (for example, EDTA), a stabalizer, an antimicrobial agent, an antifungal or antibacterial agent (for example, parabens, chlorobutanol, phenol, sorbic acid), an isotonic agent (for example, sugar, sodium chloride), a thickening agent (for example, methyl cellulose), a flavoring agent, an anti-foaming agent (for example, simethicone, Mylicon®), a disintegrant, a flow aid, a lubricant, an adjuvant, an excipient, a colorant, a diluent, a moistening agent, a preservative, a pharmaceutically compatible carrier, or a parietal cell activator.

Flavoring agents that can be used in the present invention include aspartame, thalmantin, dextrose, chocolate, vanilla, root beer, peppermint, spearmint, sucrose, cocoa, or watermelon, and the like. Other flavoring agents that may be employed include: banana, camphor, cinnamon, ginger, grape, lemon, orange, pear, apple, rum, wintergreen, acacia syrup, wild cherry, strawberry, aniseed, black currant, grapefruit, caramel, raspberry, maple, butterscotch, glycyrrhiza (licorice) syrup, citrus, walnut, lemon, tutti fruitti, cinnamon, eucalyptus, lime, orange, calcium citrate, menthol, eugenol, cylamate, xylitol, safrole, mixed berry, fruit punch, cool cherry, cool citrus, Bavarian cream, peppermint cream, cherry cream, spearmint cream, citrus cream, strawberry cream, Swiss cream, lemoni cream, mint cream, citrus punch, cola, tangerine, berry, honey, or any combination of these flavoring ingredients, for example, chocolate-mint, orange-cream, cherry-anise, lemon mint, vanilla mint, anise-menthol, honey-lemon, cherry-cinnamon, menthol eucalyptus, cinnamon-orange, or lemon-lime. In general coloring and flavoring agents should agree, for example, red for cherry, brown for chocolate. Also, effervescence may mask the salty taste of a drug. In one embodiment of the present invention, the total amount of flavoring agent may range from about 0.10 mg to about 50 mg/dosage form.

In some embodiments, the pharmaceutical composition is substantially free of sucralfate. In other embodiments of the present invention, the pharmaceutical composition is free of sucralfate. In other embodiments, the pharmaceutical composition is substantially free of amino acids. In still other embodiments, the pharmaceutical composition is free of amino acids.

In another embodiment of the present invention, the composition is in the form of a freeze dried dosage form that quickly disintegrates (for example, in less than about 10 seconds) upon contact with an aqueous media, such as when contacted with saliva in the mouth or gastric fluid. In general, a freeze dried dosage form provides for a fast dissolving agent by freeze drying a liquid suspension containing a uniformly suspended agent or agent, such as, an acid-labile pharmaceutical agent and/or a buffering agent. The basic teachings of freeze dried dosage forms are set forth in U.S. Pat. Nos. 4,371,516; 4,305,502; 4,758,598; and 4,754,597. Other examples of freeze dried dosage forms that can be utilized in the present invention are described in the following patents:

| | | | | |
|---|---|---|---|---|
| U.S. Pat. No. 4,749,790 | U.S. Pat. No. 4,894,459 | U.S. Pat. No. 4,946,684 | U.S. Pat. No. 5,021,582 | U.S. Pat. No. 5,046,618 |
| U.S. Pat. No. 5,064,946 | U.S. Pat. No. 5,075,114 | U.S. Pat. No. 8,178,867 | U.S. Pat. No. 5,188,825 | U.S. Pat. No. 5,206,025 |
| U.S. Pat. No. 5,206,072 | U.S. Pat. No. 5,215,756 | U.S. Pat. No. 5,275,823 | U.S. Pat. No. 5,457,895 | U.S. Pat. No. 5,631,023 |
| EP 90143667 | GB 1548022 | GB 2111423 | GB 211440 | GB 2119246 |
| GB 9311750 | | | | |

In one embodiment of the present invention, the general manufacturing method used to prepare a freeze dried dosage form utilizes a pre-prepared liquid composition that includes a solvent, an agent, and a gelatin containing carrier material. The liquid composition is placed into one or more shaped depressions in a tray or mold to define liquid composition filled depressions. The liquid composition in the filled depressions is frozen, then the liquid portion of the liquid composition sublimed to define a solid medicament tablet. The solid medicament filled trays are then collected. In another embodiment of the present invention, xanthan gum is added to the liquid composition, which is then stirred, prior to the freezing step. It is contemplated that xanthan gum behaves synergistically with gelatin as a flocculating agent to improve the ability of the liquid composition to suspend relatively large particles during the manufacturing process. It is also contemplated that xanthan gum has the ability to improve the suspension qualities of the liquid composition without degrading the dissolution qualities and texture of the tablet in the mouth. Examples of suitable gelatin includes plain gelatin and gelatin that is partially hydrolyzed, for example by heating gelatin in water. Examples of other suitable carrier materials that can be combined with gelatin are those that are inert and pharmaceutically acceptable for use in preparing pharmaceutical dosage forms. Such carrier materials include polysaccharides such as dextran and polypeptides.

In one embodiment of the present invention, the agent used in a freeze-dried dosage form includes a buffering agent having an average particle size ranging from about 1 μm to about 400 μm. Any particulate agent that remains at least partially in the solid state in the matrix of the carrier material may be used in the present invention. In yet another embodiment of the present invention, the freeze dried dosage form contains an enteric-coated acid-labile pharmaceutical agent, such as, a proton pump inhibiting agent.

In yet another embodiment, the proton pump inhibiting agent is lyophilized to obtain a freeze-drying of an aqueous solution of the agent for inclusion into a composition of the present invention. One such freeze drying technique that can be used in the present invention is described in, for example, U.S. patent application. No. 2003/0003058, which describes lyophilized pantoprazole, ethylenediamine tetraacetic acid, and/or a suitable salt thereof, and sodium hydroxide and/or sodium carbonate.

In still another example, a pharmaceutical formulation is prepared by mixing enteric-coated granules of a proton pump inhibiting agent with one or more buffering agents (for example, omeprazole 20 mg granules plus 500 mg sodium bicarbonate and 500 mg calcium carbonate) in a solid dosage form. Upon oral administration, the buffering agents elevate the gastric pH such that all or part of the enteric-coating is dissolved in the gastric fluid (rather than, for example, in the higher pH environment of the duodenum), and the omeprazole is available for immediate release in the gastric fluid for absorption into the bloodstream. Many variations in this type of formulation (that is, higher or lower amounts of inhibiting agent and/or buffering agent) may be utilized in the present invention.

The pharmaceutical composition of the invention comprises a buffering agent, which can be any suitable buffering agent that, when formulated or delivered before, during and/or after the proton pump inhibiting agent, functions to substantially prevent or inhibit the acid degradation of at least some of the proton pump inhibiting agent by gastric acid sufficient to preserve the bioavailability of the proton pump inhibiting agent administered. Suitable buffering agents include, for example, buffering agents as described herein, such as sodium salts, potassium salts, magnesium salts, and calcium salts, or any other suitable buffering agent or mixture of buffering agents.

The buffering agent is administered in an amount sufficient to substantially prevent or inhibit the acid degradation of at least some of the proton pump inhibiting agent by gastric acid sufficient to preserve the bioavailability of a therapeutically effective amount of the proton pump inhibiting agent administered, thus preserving the ability of the proton pump inhibiting agent to elicit a therapeutic effect. Therefore, the amount of buffering agent of the compositions of the present invention, when in the presence of the biological fluids of the stomach, must only elevate the pH of these biological fluids sufficiently to achieve adequate bioavailability of the drug to effect therapeutic action.

In one embodiment, the buffering agent is present in the methods, kits, combinations, and compositions of the present invention in an amount of about 0.05 mEq to about 10.0 mEq per mg of proton pump inhibiting agent. In another embodiment of the present invention the buffering agent is present in an amount of about 0.2 mEq to about 5 mEq per mg of the proton pump inhibiting agent. Illustratively, the amount of the buffering agent in the composition is about 0.2 mEq, or about 1 mEq, or about 2 mEq, or about 3 mEq, or about 5 mEq, or about 10 mEq, or about 11 mEq, or about 12.5 mEq, or about 13 mEq, or about 15 mEq, or about 19 mEq, or about 20 mEq, or about 21 mEq, or about 22 mEq, or about 23 mEq, or about 24 mEq, or about 25 mEq, or about 30 mEq, or about 31 mEq, or about 35 mEq, or about 40 mEq, or about 45 mEq, or about 50 mEq, or about 55 mEq, or about 60 mEq, or about 65 mEq, or about 70 mEq, or about 75 mEq, 80 mEq, or about 90 mEq, or about 100 mEq, or about 110 mEq, or about 120 mEq, or about 130 mEq, or about 140 mEq, or about 150 mEq, or about 160 mEq per dose.

In yet another embodiment of the present invention the buffering agent is present in an amount of at least 10 mEq. In yet another embodiment of the present invention the buffering agent is present in an amount of about 5 mEq to about 70 mEq. In still another embodiment, the buffering agent is present in an amount of about 20 mEq to about 40 mEq. And in yet another embodiment of the present invention, the amount of the buffering agent is present in an amount more than about 20 times, or more than 22 times, or more than 25 times, or more than about 30 times, or more than 35 times, or more than about 40 times the amount of the proton pump inhibiting agent on a weight to weight basis in the composition. The specific mEq amounts of buffer can vary, for example, from between about 0.01% to about 20% or more, depending on the application and desired therapeutic result.

In another aspect of the invention, compositions are provided wherein the amount of buffering agent present in the pharmaceutical composition is between 200 and 3500 mg. In some embodiments, the amount of buffering agent present in the pharmaceutical composition is about 200 mg, or about 300 mg, or about 400 mg, or about 500 mg, or about 600 mg, or about 700 mg, or about 800 mg, or about 900 mg, or about 1000 mg, or about 1100 mg, or about 1200 mg, or about 1300 mg, or about 1400 mg, or about 1500 mg, or about 1600 mg, or about 1700 mg, or about 1800 mg, or about 1900 mg, or about 2000 mg, or about 2100 mg, or about 2200 mg, or about 2300 mg, or about 2400 mg, or about 2500 mg, or about 2600 mg, or about 2700 mg, or about 2800 mg, or about 2900 mg, or about 3000 mg, or about 3200 mg, or about 3500 mg.

In one embodiment of the present invention, the buffering agent is sodium carbonate and is present in the methods, kits, combinations and compositions in an amount of at least about 250 mg. In another embodiment, the sodium carbonate is present in an amount of at least about 700 mg. In yet another embodiment, the sodium carbonate is present in an amount from about 250 mg to about 4000 mg. In still another embodiment, the sodium carbonate is present in an amount from about 1000 mg to about 2000 mg. And in still another embodiment, the sodium carbonate is present in an amount from about 1250 mg to about 1750 mg. Illustratively, the amount of buffering agent in a composition of the present invention is about 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, or 1750 mg. These specific amounts can vary, for example, from between about 0.01% to about 20% or more, depending on the application and desired therapeutic result.

In one embodiment of the present invention, the buffering agent is calcium carbonate and is present in the methods, kits, combinations and compositions in an amount of at least about 250 mg. In another embodiment, the calcium carbonate is present in an amount of at least about 700 mg. In yet another embodiment, the calcium carbonate is present in an amount from about 250 mg to about 4000 mg. And in still another embodiment, the calcium carbonate is present in an amount from about 500 mg to about 1500 mg. Illustratively, the amount of buffering agent in a composition of the present invention is about 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, or 1750 mg. These specific amounts can vary, for example, from between about 0.01% to about 20% or more, depending on the application and desired therapeutic result.

In one embodiment of the present invention, the buffering agent is sodium bicarbonate and calcium carbonate present in the methods, kits, combinations and compositions in an amount totaling at least about 250 mg. In another embodiment, the sodium bicarbonate and calcium carbonate are present in an amount totaling at least about 700 mg. In yet another embodiment, the sodium bicarbonate and calcium carbonate are present in an amount totaling from about 250 mg to about 4000 mg. In still another embodiment, the sodium bicarbonate is present in an amount from about 1000 mg to about 2000 mg. And in still another embodiment, the sodium bicarbonate is present in an amount from about 1250 mg to about 1750 mg. Illustratively, the amount of buffering agent in a composition of the present invention is about 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, or 1750 mg. These specific amounts can vary, for example, from between about 0.01% to about 20% or more, depending on the application and desired therapeutic result.

Compositions are provided as described herein, wherein the buffering agent is present in an amount of about 0.1 mEq/mg to about 5 mEq/mg of the proton pump inhibitor, or about 0.25 mEq/mg to about 3 mEq/mg of the proton pump inhibitor, or about 0.3 mEq/mg to about 2.5 mEq/mg of the proton pump inhibitor, or about 0.4 mEq/mg to about 2.0 mEq/mg of the proton pump inhibitor, or about 0.5 mEq/mg to about 1.5 mEq/mg of the proton pump inhibitor. Compositions are provided as described herein, wherein the buffering agent is present in an amount of at least 0.25 mEq/mg to about 2.5 mEq/mg of the proton pump inhibitor, or at least about 0.4 mEq/mg of the proton pump inhibitor.

Microencapsulation and Coatings

All or part of the proton pump inhibitor of the present invention may or may not be enteric-coated, or in a sustained-release or delayed-release form, depending on the context in which the proton pump inhibiting agent is utilized. In one embodiment of the present invention the proton pump inhibiting agent is not enteric-coated, or coated with a sustained-release or delayed-release coating. In yet another embodiment the proton pump inhibitor is enteric-coated, or coated with a sustained-release or delayed-release coating. And in another embodiment the composition may contain both an enteric-coated proton pump inhibiting agent and a non-enteric-coated proton pump inhibiting agent. Such a composition is contemplated where both an immediate release of the proton pump inhibiting agent into the gastric fluid, for example, an absorption pool of a subject, is desired as well as a delayed-release of the proton pump inhibiting agent providing an extended therapeutic effect.

In some embodiments of the present invention all or part of the proton pump inhibitor is microencapsulated with a material that enhances the shelf-life of the pharmaceutical compositions. Exemplary microencapsulation materials useful for enhancing the shelf-life of pharmaceutical compositions comprising a proton pump inhibitor include, but are not limited to: cellulose hydroxypropyl ethers (HPC) such as Klucel® or Nisso HPC; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, Benecel MP824, and Benecel MP843; methylcellulose polymers such as Methocel® and Metolose®; Ethylcelluloses (EC) and mixtures thereof such as E461, Ethocel®, Aqualon®-EC, Surelease®; Polyvinyl alcohol (PVA) such as Opadry AMB; hydroxyethylcelluloses such as Natrosol®; carboxymethylcelluloses and salts of carboxymethylcelluloses (CMC) such as Aqualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins; and mixtures of these materials. In other embodiments, some or all of the antacid is microencapsulated with a material that enhances the shelf-life of the pharmaceutical composition. In various embodiments, a buffering agent such as sodium bicarbonate is incorporated into the microencapsulation material. In other embodiments, an antioxidant such as BHT is incorporated into the microencapsulation material. In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for enhancing the shelf-life of the pharmaceutical compositions is from the USP or the National Formulary (NF).

In some embodiments, all or some of the proton pump inhibitor is coated. In other embodiments, all or some of the antacid is coated. The coating useful in the present invention may be, for example, a gastric resistant coating such as an enteric coating, a controlled-release coating, an enzymatic-controlled coating, a film coating, a sustained-release coating, an immediate-release coating, or a delayed-release coating. According to another aspect of the invention, the coating may be useful for enhancing the stability of the pharmaceutical compositons of the present invention.

Various techniques may be used to determine whether a pharmaceutical composition has an enhanced shelf-life. For example, a pharmaceutical composition of the present invention may have an enhanced shelf-life stability if the pharmaceutical composition contains less than about 5% total impurities after about 3 years of storage, or after about 2.5 years of storage, or about 2 years of storage, or about 1.5 years of storage, or about 1 year of storage, or after 11 months of storage, or after 10 months of storage, or after 9 months of storage, or after 8 months of storage, or after 7 months of storage, or after 6 months of storage, or after 5 months of storage, or after 4 months of storage, or after 3 months of storage, or after 2 months of storage, or after 1 month of storage.

Micronized Proton Pump Inhibitor

Particle size of the proton pump inhibitor can affect the solid dosage form in numerous ways. Since decreased particle size increases in surface area (S), the particle size reduction provides an increase in the rate of dissolution (dM/dt) as expressed in the Noyes-Whitney equation below:

dM/dt=dS/h(Cs−C)

M=mass of drug dissolved; t=time; D=diffusion coefficient of drug; S=effective surface area of drug particles; H=stationary layer thickness; Cs=concentration of solution at saturation; and C=concentration of solution at time t.

Because omeprazole, as well as other proton pump inhibitors, has poor water solubility, to aid the rapid absorption of the drug product, various embodiments of the present invention use micronized proton pump inhibitor is used in the drug product formulation.

In some embodiments, the average particle size of at least about 90% the micronized proton pump inhibitor is less than about 40 μm, or less than about 35 μm, or less than about 30 μm, or less than about 25 μm, or less than about 20 μm, or less than about 15 μm, or less than about 10 μm. In other embodiments, at least 80% of the micronized proton pump inhibitor has an average particle size of less than about 40 μm, or less than about 35 μm, or less than about 30 μm, or less than about 25 μm, or less than about 20 μm, or less than about 15 μm, or less than about 10 μm. In still other embodiments, at least 70% of the micronized proton pump inhibitor has an average particle size of less than about 40 μm, or less than about 35 μm, or less than about 30 μm, or less than about 25 μm, or less than about 20 μm, or less than about 15 μm, or less than about 1.0 μm.

Compositions are provided wherein the micronized proton pump inhibitor is of a size which allows greater than 75% of the proton pump inhibitor to be released within about 1 hour, or within about 50 minutes, or within about 40 minutes, or within about 30 minutes, or within about 20 minutes, or within about 10 minutes or within about 5 minutes of dissoluion testing. In another embodiment of the invention, the micronized proton pump inhibitor is of a size which allows greater than 90% of the proton pump inhibitor to be released within about 1 hour, or within about 50 minutes, or within about 40 minutes, or within about 30 minutes, or within about 20 minutes, or within about 10 minutes or within about 5 minutes of dissoluion testing.

Administration

The present invention provides a pharmaceutical composition comprising a proton pump inhibiting agent and a buffering agent for oral administration by a subject. In one embodiment, upon administration to a fed subject, the composition contacts the gastric fluid of the stomach and increases the gastric pH of the stomach to a pH that prevents or inhibits acid degradation of the proton pump inhibiting agent in the gastric fluid of the stomach and allows a measurable serum concentration of the proton pump inhibiting agent to be absorbed into the blood serum of the subject, such that pharmacokinetic and pharmacodynamic parameters can be obtained using testing procedures known to those skilled in the art.

The present invention also provides a pharmaceutical composition comprising a proton pump inhibiting agent and a buffering agent for oral administration and ingestion by a subject that exhibits increased omeprazole bioavailability when administered to a fed subject compared with administration to a fasting subject on the first day of administration. The present invention further provides pharmaceutical compositions that exhibit a decreased omeprazole bioavailability when administered to a fed human subject compared with administration to a fasting adult human subject on the seventh consecutive day of daily administration.

Thus, the present invention provides a pharmaceutical composition comprising a proton pump inhibiting agent and a buffering agent for oral administration and ingestion by a subject. The pharmaceutical compositions can be administered to a subject at any time in relation to the ingestion of food, for example, to a fed subject or to a fasting subject.

A fed subject can be, for example, a subject who is initiating ingestion of a meal, a subject who has initiated ingestion of a meal a short time before administration (e.g., at about 10 minutes before, at about 20 minutes before, at about 30 minutes before, at about 45 minutes before, at about 60 minutes before, or at about 90 minutes before, or at about 120 minutes before), a subject who has initiated ingestion of a meal a short time before administration and continues to ingest food after administration, a subject who has recently finished ingesting a meal, or a subject who has finished ingesting a meal and who is experiencing symptoms related to the ingestion of that meal. A meal can be any amount of food, for example, a snack, a serving of food, several servings of one food, one or several servings each of different foods, or any amount of food that induces symptoms necessitating treatment with a proton pump inhibitor.

Pharmaceutical compositions of the present invention may also be administered to a fasting subject. A fasting subject can be any subject who has abstained from food for a period of time, e.g., a subject who has not ingested a meal overnight (e.g., 8 hours), a subject who has not ingested a meal in several hours, a subject with an empty stomach who is not suffering any meal-related symptoms that can be treated with a proton pump inhibitor, or any subject who has not ingested a meal such that the most recently ingested meal is digested and the subject is not suffering from any meal-related symptoms that can be treated with a proton pump inhibitor.

In one embodiment, upon administration to a fed subject, the composition contacts the gastric fluid of the stomach and increases the gastric pH of the stomach to a pH that prevents or inhibits acid degradation of the proton pump inhibiting agent in the gastric fluid of the stomach and allows a measurable serum concentration of the proton pump inhibiting agent to be absorbed into the blood serum of the subject, such that pharmacokinetic and pharmacodynamic parameters can be obtained using testing procedures known to those skilled in the art.

In one embodiment, the pharmaceutical composition of the invention exhibits increased omeprazole bioavailability when administered to a fed subject compared with administration to a fasting subject on the first day of administration. In another embodiment, the pharmaceutical composition exhibits a decreased omeprazole bioavailability when administered to a fed human subject compared with administration to a fasting adult human subject on the seventh consecutive day of daily administration.

The present invention is also directed to methods of treating a condition or disorder by administering the pharmaceutical composition of the invention where treatment with an inhibitor of H+, K+-ATPase is indicated. The condition or disorder can be, for example, an acid-caused gastrointestinal disorder such as, e.g., heartburn, duodenal ulcer disease, a gastric ulcer disease, a gastroesophageal reflux disease, erosive esophagitis, a poorly responsive symptomatic gastroesophageal reflux disease, a pathological gastrointestinal hypersecretory disease, Zollinger Ellison Syndrome, or acid dyspepsia.

A pharmaceutical formulation of the proton pump inhibiting agents utilized in the present invention can be administered orally or internally to the subject. This can be accomplished, for example, by administering the solution via a nasogastric (ng) tube or other indwelling tubes placed in the GI tract. In one embodiment of the present invention, in order to avoid the disadvantages associated with administering large amounts of sodium bicarbonate, the proton pump inhibiting agent solution of the present invention is administered in a single dose which does not require any further administration of bicarbonate, or other buffer following the administration of the proton pump inhibiting agent solution, nor does it require a large amount of bicarbonate or buffer in total. That is, unlike the proton pump inhibiting agent solutions and administration protocols outlined above in the Background of the Invention section, a formulation of the present invention is given in a single dose, which does not require administration of bicarbonate either before or after administration of the proton pump inhibiting agent. The present invention eliminates the need to pre- or post-dose with additional volumes of water and sodium bicarbonate. The amount of bicarbonate administered via the single dose administration of the present invention is less than the amount of bicarbonate administered as taught in the references cited above.

Embodiments of the present invention also provide pharmaceutical compositions wherein a therapeutically effecitive dose of the proton pump inhibitor is in the blood serum of the patient within about 45 minutes, or within about 30 minutes, or within about 25 minutes, or within about 20 minutes, or within about 15 minutes, or within about 10 minutes, or within about 5 minutes after ingestion of the pharmaceutical composition.

In various embodiments of the present invention, the pH of the stomach is increased to a pH about 3, or a pH above 3.5, or a pH above 4, or a pH above 4.5, or a pH above 5, or a pH above 5.5, or a pH above 6, or a pH above 6.5, or a pH above 7 within about 45 minutes after administration of the pharmaceutical composition. In other embodiments of the present invention, the pH of the stomach is increased to a pH about 3, or a pH above 3.5, or a pH above 4, or a pH above 4.5, or a pH above 5, or a pH above 5.5, or a pH above 6, or a pH above 6.5, or a pH above 7 within about 30 minutes after administration of the pharmaceutical composition. In still other embodiments, the pH of the stomach is increased to a pH about 3, or a pH above 3.5, or a pH above 4, or a pH above 4.5, or a pH above 5, or a pH above 5.5, or a pH above 6, or a pH above 6.5, or a pH above 7 within about 15 minutes after administration of the pharmaceutical composition.

Dosing

The proton pump inhibiting agent is administered and dosed in accordance with good medical practice, taking into account the clinical condition of the individual patient, the site and method of administration, scheduling of administration, and other factors known to medical practitioners. In human therapy, it is important to provide a dosage form that delivers the required therapeutic amount of the drug in vivo, and renders the drug bioavailable in a rapid manner. In addition to the dosage forms described herein, the dosage forms described in Phillips, U.S. Pat. Nos. 5,840,737; 6,489,346; and 6,645,988 are incorporated herein by reference.

Besides being useful for human treatment, the present invention is also useful for veterinary treatment of mammals, reptiles, birds, exotic animals and farm animals, including mammals, rodents, and the like. In one embodiment, the mammal includes a primate, for example, a human, a monkey, or a lemur, a horse, a dog, a pig, or a cat. In another embodiment, the rodent includes a rat, a mouse, a squirrel or a guinea pig.

In one embodiment of the present invention, the composition is administered to a subject in a therapeutically-effective amount, that is, the composition is administered in an amount that achieves a therapeutically-effective dose of a proton pump inhibiting agent in the blood serum of a subject for a period of time to elicit a desired therapeutic effect. Illustratively, in a fed adult human the composition is administered to achieve a therapeutically-effective dose of a proton pump inhibiting agent in the blood serum of a subject within about 5 minutes after administration of the composition. In another embodiment of the present invention, a therapeutically-effective dose of the proton pump inhibiting agent is achieved in the blood serum of a subject within about 10 minutes from the time of administration of the composition to the subject. In another embodiment of the present invention, a therapeutically-effective dose of the proton pump inhibiting agent is achieved in the blood serum of a subject within about 20 minutes from the time of administration of the composition to the subject. In yet another embodiment of the present invention, a therapeutically-effective dose of the proton pump inhibiting agent is achieved in the blood serum of a subject within about 30 minutes from the time of administration of the composition to the subject. In still another embodiment of the present invention, a therapeutically-effective dose of the proton pump inhibiting agent is achieved in the blood serum of a subject within about 40 minutes from the time of administration of the composition to the subject.

In one embodiment of the present invention, a therapeutically-effective dose of the proton pump inhibiting agent is achieved in the blood serum of a subject within about 20 minutes to about 12 hours from the time of administration of the composition to the subject. In another embodiment of the present invention, a therapeutically-effective dose of the proton pump inhibiting agent is achieved in the blood serum of a subject at about 20 minutes to about 6 hours from the time of administration of the composition to the subject. In yet another embodiment of the present invention, a therapeutically-effective dose of the proton pump inhibiting agent is achieved in the blood serum of a subject at about 20 minutes to about 2 hours from the time of administration of the composition to the subject. In still another embodiment of the present invention, a therapeutically-effective dose of the proton pump inhibiting agent is achieved in the blood serum of a subject at about 40 minutes to about 2 hours from the time of administration of the composition to the subject. And in yet another embodiment of the present invention, a therapeutically-effective dose of the proton pump inhibiting agent is achieved in the blood serum of a subject at about 40 minutes to about 1 hour from the time of administration of the composition to the subject.

In general, a composition of the present invention is administered at a dose suitable to provide an average blood serum concentration of a proton pump inhibiting agent of at least about 1.0 μg/ml in a subject over a period of about 1 hour after administration. Contemplated compositions of the present invention provide a therapeutic effect as proton pump inhibiting agent medications over an interval of about 5 minutes to about 24 hours after administration, enabling once-a-day or twice-a-day administration if desired. In one embodiment of the present invention, the composition is administered at a dose suitable to provide an average blood serum concentration of a proton pump inhibiting agent of at least about 1.0 μg/ml in a subject about 10 minutes, or about 20 minutes, or about 30 minutes, or about 40 minutes after administration of the composition to the subject.

In one embodiment of the present invention, the composition is administered in an amount to achieve a measurable serum concentration of the proton pump inhibiting agent greater than about 0.1 μg/ml within about 15 minutes after administration of the composition. In another embodiment of the present invention, the composition is administered in an amount to achieve a measurable serum concentration of the proton pump inhibiting agent greater than about 0.1 μg/ml within about 30 minutes after administration of the composition. In other embodiments contemplated by the present invention, the composition is administered in an amount to achieve a measurable serum concentration of the proton pump inhibiting agent greater than about 0.1 μg/ml within about 45 minutes after administration of the composition. In another embodiment of the present invention, the composition is administered to the subject in an amount to achieve a measurable serum concentration of the proton pump inhibiting agent greater than about 0.1 μg/ml from about 15 minutes to about 6 hours after administration of the composition.

In yet another embodiment of the present invention, the composition is administered to the subject in an amount to achieve a measurable serum concentration of the proton pump inhibiting agent greater than about 0.15 μg/ml from about 15 minutes to about 1.5 hours after administration of the composition.

In still another embodiment of the present invention, the composition is administered to the subject in an amount to achieve a measurable serum concentration of the proton pump inhibiting agent greater than about 0.2 μg/ml within about 15 minutes after administration of the composition.

In one embodiment, substantially the entire dose of the pharmaceutical agent is released from the composition of the present invention into gastric fluid within less than about 120 minutes, or within about 1 minute to about 120 minutes, or within about 2 minutes, or within about 5 minutes, or within about 10 minutes, or within about 20 minutes, or within about 30 minutes, or within about 40 minutes, or within about 80 minutes, or within about 120 minutes.

In one embodiment, the pharmaceutical composition comprises an amount of buffering agent sufficient to increase the pH of the gastric fluid to a target pH for a period of time. Where the gastric fluid is the stomach of a subject, the period of time is generally sufficient for the pharmaceutical agent to be absorbed into the blood stream. Illustratively, the pH is about 3 to about 8, or greater than about 3, or about 3.5, or about 4, or about 4.5, or about 5, or about 5.5, or about 6, or about 6.5, or about 7, or about 7.5, or about 8. The particular target pH can depend, among other things, on the particular pharmaceutical agent utilized in the composition, and its acid labile characteristics (for example, its pKa).

In yet another embodiment, the pH of the gastric fluid is maintained for a time period that substantially dissolves an enteric-coating covering some or all of the proton pump inhibitor. Illustratively, the time period is about less than about 120 minutes, or about 30 seconds to about 120 minutes, or greater than about 1 minute, or greater than about 2 minutes, or greater than about 5 minutes, or greater than about 10 minutes, or greater than about 15 minutes, or greater than about 20 minutes, or greater than about 30 minutes, or greater than about 40 minutes, or greater than about 50 minutes, or greater than about 60 minutes, or greater than about 90 minutes, or greater than about 120 minutes.

In order to measure and determine the gastrointestinal disorder- or disease-effective amount of a proton pump inhibiting agent to be delivered to a subject, serum proton pump inhibiting agent concentrations can be measured using standard assay techniques.

The amount of therapeutic agent necessary to elicit a therapeutic effect can be experimentally determined based on, for example, the absorption rate of the agent into the blood serum, the bioavailability of the agent, and the amount of protein binding of the agent. It is understood, however, that specific dose levels of the therapeutic agents of the present invention for any particular patient depends upon a variety of factors including the activity of the specific compound employed, the age, body weight, general health, sex, and diet of the subject (including, for example, whether the subject is in a fasting or fed state), the time of administration, the rate of excretion, the drug combination, and the severity of the particular disorder being treated and form of administration. Fed state generally refers to the period of time of initial ingestion of food by a subject through about 30 minutes to about 4 hours after completing a meal. Treatment dosages generally may be titrated to optimize safety and efficacy.

Typically, dosage-effect relationships from in vitro and/or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route administered, the condition of the particular subject, etc. Generally speaking, one will desire to administer an amount of the compound that is effective to achieve a serum level commensurate with the concentrations found to be effective in vitro for a period of time effective to elicit a therapeutic effect. Thus, where a compound is found to demonstrate in vitro activity at, for example, 10 ng/ml, one will desire to administer an amount of the drug that is effective to provide at least about a 10 ng/ml concentration in vivo for a period of time that elicits a desired therapeutic effect, for example, raising of gastric pH, reducing gastrointestinal bleeding, reducing the need for blood transfusion, improving survival rate, more rapid recovery, parietal cell activation and H⁺,K⁺-ATPase inhibition or improvement or elimination of symptoms, and other indicators as are selected as appropriate measures by those skilled in the art. Determination of these parameters is well within the skill of the art. These considerations are well known in the art and are described in standard textbooks.

It will be understood that the amount of proton pump inhibiting agent and/or buffering agent that is administered to a subject is dependent on, for example, the sex, general health, et, and/or body weight of the subject. Illustratively, where the agent is a substituted benzimidazole such as, for example, omeprazole, lansoprazole, pantoprazole, rabeprazole, esomeprazole, pariprazole, or leminoprazole, and the subject is, for example, a child or a small animal (for example, a dog), a relatively low amount of the agent in the dose range of about 1 mg to about 60 mg is likely to provide blood serum concentrations consistent with therapeutic effectiveness. Where the subject is an adult human or a large animal (for example, a horse), achievement of such blood serum concentrations of the agent are likely to require dose units containing a relatively greater amount of the agent, for example, a 5 mg, 10 mg, 15 mg, 20 mg, 25 mg, 30 mg, 35 mg, 40 mg, 45 mg, 50 mg, 55 mg, 60 mg, 65 mg, 70 g, 80 mg, 85 mg, 90 mg, 95 mg, 100 mg, 105 mg, 110 mg, 115 mg, or 120 mg dose for an adult human, or a 150 mg, 200 mg, 400 mg, 800 mg, or 1000 mg dose for an adult horse.

The solid compositions of the present invention are generally in the form of discrete unit dosage forms, such as in a tablet (for example, a suspension tablet, chewable tablet, a caplet, or effervescent tablet), pill, powder (for example, a sterile packaged powder, dispensable powder, effervescent powder), capsule (for example, a soft or hard gelatin capsule), lozenge, sachet, cachet, troche, pellet, or granule. Such unit dosage forms typically contain about 1 mg to about 1000 mg of the proton pump inhibiting agent, or about 5 mg to about 240 mg, or about 10 mg to about 160 mg, or about 15 mg to about 120 mg, or about 20 mg to about 80 mg. Illustratively, these unit dose articles may contain about a 2 mg, or about a 5 mg, or about a 10 mg, or about a 15 mg, or about a 20 mg, or about a 25 mg, or about a 30 mg, or about a 35 mg, or about a 40 mg, or about a 45 mg, or about a 50 mg, or about a 55 mg, or about a 60 mg, or about a 65 mg, or about a 70 mg, or about a 75 mg, or about a 80, mg, or about a 85 mg, or about a 90 mg, or about a 95 mg, or about a 100 mg, or about a 110 mg, or about a 120 mg, or about a 130 mg, or about a 140 mg, or about a 150 mg, or about a 160 mg, or about a 170 mg, or about a 180 mg, or about a 190 mg, or about a 200 mg, or about a 220 mg, or about a 240 mg dose of a proton pump inhibiting agent.

In one embodiment, the buffering agent is present in compositions of the present invention in an amount of about 0.05 mEq to about 10.0 mEq per mg of proton pump inhibiting agent, or about 0.1 mEq to about 2.5 mEq per mg of proton pump inhibiting agent, or about 0.4 mEq to about 1.0 mEq per mg of proton pump inhibiting agent. Such dosage units may be given at least once, twice, three, or four times a day, or as many times as needed to elicit a therapeutic response. A particular unit dosage form can be selected to accommodate the desired frequency of administration used to achieve a specified daily dosage.

Pharmacokinetic and Pharmacodynamic Measurements

The present invention provides a pharmaceutical composition comprising a proton pump inhibiting agent and a buffering agent for oral administration and ingestion by a subject. In one embodiment, upon administration to a fed subject, the composition contacts the gastric fluid of the stomach and increases the gastric pH of the stomach to a pH that prevents or inhibits acid degradation of the proton pump inhibiting agent in the gastric fluid of the stomach and allows a measurable serum concentration of the proton pump inhibiting agent to be absorbed into the blood serum of the subject, such that the composition exhibits one component of a pharmacokinetic or pharmacodynamic profile.

The present invention also provides a pharmaceutical composition comprising a proton pump inhibiting agent and a buffering agent for oral administration and ingestion by a subject that exhibits increased omeprazole bioavailability when administered to a fed subject compared with administration to a fasting subject on the first day of administration, such that the composition exhibits one component of a pharmacokinetic or pharmacodynamic profile. The present invention further provides a pharmaceutical composition that exhibit a decreased omeprazole bioavailability when administered to a fed human subject compared with administration to a fasting adult human subject on the seventh consecutive day of daily administration, such that the composition exhibits one component of a pharmacokinetic or pharmacodynamic profile.

In one embodiment, a solid pharmaceutical composition of the present invention comprises a gastrointestinal-disorder amount of at least one proton pump inhibiting agent and at least one buffering agent, and upon oral administration to a fed human subject, exhibits at least one component of a proton pump inhibiting agent pharmacokinetic profile and/or a proton pump inhibiting agent pharmacodynamic profile. In one embodiment, the proton pump inhibiting agent pharmacokinetic profile has at least one of(i) a Cₘₐₓ not less than about 880 ng/ml; (ii) a Tₘₐₓ not greater than about 1.5 hours; (iii) an AUC_{(0-inf)} not less than about 3860 ng×hr/ml; or (iv) a plasma proton pump inhibiting agent concentration about one hour after administration not less than about 750 ng/ml. In yet another embodiment, the proton pump inhibiting agent pharmacodynamic profile has at least one of (i) an integrated acidity of not greater than about 0 mmol×hr/L; (ii) an integrated acidity of not greater than about 11.1 mmol×hr/L; (iii) an integrated acidity of not greater than about 41.5 mmol×hr/L; or (ii) an increased pH above 4.0 for at least about 4 hours to about 5 hours after ingestion of a meal at about 160 minutes after the oral administration.

In still another embodiment of the present invention, a pharmaceutical composition comprises omeprazole and sodium bicarbonate, where the composition is orally administered to a fed adult human subject, and exhibits an omeprazole bioavailability AUC_{(0-inf)} at least about 45% to about 75% greater than the omeprazole bioavailability exhibited by administration of either omeprazole without the sodium bicarbonate to a fasting adult human subject on the first day of administration of the dosage amount to the fasting subject, or oral administration of an enteric-coated omeprazole delayed-release capsule to a fasting adult human subject on the first day of administration of the capsule to the fasting subject.

In yet another embodiment of the present invention, a pharmaceutical composition comprises omeprazole and sodium bicarbonate, wherein the composition is orally administered to a fed adult human subject, and exhibits an omeprazole pharmacokinetic profile having at least one parameter of a described AUC_{(0-inf)} and/or a Cₘₐₓ. In one embodiment, the AUC_{(0-inf)} is at least about 18% less than an AUC_{(0-inf)} exhibited by oral administration of omeprazole without sodium bicarbonate to a fasting adult human subject and/or by oral administration of an omeprazole delayed-release enteric-coated capsule to a fasting adult human subject. In yet another embodiment, the Cₘₐₓ is at least about 45% to about 55% less than a Cₘₐₓ exhibited by oral administration of omeprazole without sodium bicarbonate to a fasting adult human subject and/or by oral administration of an enteric-coated omeprazole delayed-release capsule to a fasting adult human subject.

In still another embodiment of the present invention, a method of preparing an oral dosage form by dry mixing at least one proton pump inhibiting agent and at least one buffering agent to form a mixture into the oral dosage form is provided. The dosage form when orally administered to a fed human subject, exhibits at least one component of a proton pump inhibiting agent pharmacokinetic profile and/or a proton pump inhibiting agent pharmacodynamic profile. In one embodiment, the proton pump inhibiting agent pharmacokinetic profile has at least one of (i) a Cₘₐₓ not less than about 880 ng/ml; (ii) a Tₘₐₓ not greater than about 1.5 hours; (iii) an AUC_{(0-inf)} not less than about 3860 ng×hr/ml; or (iv) a plasma proton pump inhibiting agent concentration about one hour after administration not less than about 750 ng/ml. In yet another embodiment, the proton pump inhibiting agent pharmacodynamic profile has at least one of (i) an integrated acidity of not greater than about 0 mmol×hr/L; (ii) an integrated acidity of not greater than about 11.1 mmol×hr/L; (iii) an integrated acidity of not greater than about 41.5 mmol×hr/L; or (ii) an increased pH above 4.0 for at least about 4 hours to about 5 hours after ingestion of a meal at about 160 minutes after the oral administration.

Pharmacokinetic and pharmacodynamic data can be obtained by known techniques in the art. Due to the inherent variation in pharmacokinetic and pharmacodynamic parameters of drug metabolism in human subjects, appropriate pharmacokinetic and pharmacodynamic profile components describing a particular composition can vary. Typically, pharmacokinetic and pharmacodynamic profiles are based on the determination of the “mean” parameters of a group of subjects. The group of subjects include any reasonable number of subjects suitable for determining a representative mean, for example, 5 subjects, 10 subjects, 16 subjects, 20 subjects, 25 subjects, 30 subjects, 35 subjects, or more. The “mean” is determined by calculating the average of all subject's measurements for each parameter measured.

The pharmacokinetic parameters can be any parameters suitable for describing the present composition. For example, the Cₘₐₓ can be not less than about 500 ng/ml; not less than about 550 ng/ml; not less than about 600 ng/ml; not less than about 700 ng/ml; not less than about 800 ng/ml; not less than about 880 ng/ml, not less than about 900 ng/ml; not less than about 100 ng/ml; not less than about 1250 ng/ml; not less than about 1500 ng/ml, not less than about 1700 ng/ml, or any other Cₘₐₓ appropriate for describing the proton pump inhibiting agent pharmacokinetic profile. The Tₘₐₓ can be, for example, not greater than about 0.5 hours, not greater than about 1.0 hours, not greater than about 1.5 hours, not greater than about 2.0 hours, not greater than about 2.5 hours, or not greater than about 3.0 hours, or any other Tₘₐₓ appropriate for describing the proton pump inhibiting agent pharmacokinetic profile. The AUC_{(0-inf)} can be, for example, not less than about 590 ng×hr/ml, not less than about 1500 ng×hr/ml, not less than about 2000 ng×hr/ml, not less than about 3000 ng×hr/ml, not less than about 3860 ng×hr/ml, not less than about 4000 ng×hr/ml, not less than about 5000 ng/ml, not less than about 6000 ng x hr/ml, not less than about 7000 ng×hr/ml, not less than about 8000 ng×hr/ml, not less than about 9000 ng×hr/ml, or any other AUC(0 inf) appropriate for describing the proton pump inhibiting agent pharmacokinetic profile of the inventive composition. The plasma omeprazole concentration about one hour after administration can be, for example, not less than about 140 ng/ml, not less than about 425 ng/mil, not less than about 550 ng/ml, not less than about 640 ng/ml, not less than about 720 ng/ml, not less than about 750 ng/ml, not less than about 800 ng/ml, not less than about 900 ng/ml, not less than about 1000 ng/ml, not less than about 1200 ng/ml, or any other plasma proton pump inhibiting agent concentration suitable for describing the inventive composition.

The pharmacodynamic parameters can be any parameters suitable for describing the present composition. For example, the pharmacodynamic profile can exhibit an integrated acidity of not greater than, for example, about 20 mmol×hr/L, about 30 mmol×hr/L, about 41.5 mmol×hr/L, about 50 mmol×hr/L, about 60 mmol×hr/L, or any other integrated acidity appropriate for describing the inventive composition. The pharmacodynamic profile can exhibit an increased pH above 4.0 for, for example, at least about 2 hours, at least about 3 hours, at least about 4 hours, at least about 4 to about 5 hours, at least about 5 hours, at least about 6 hours, at least about 7 hours, at least about 8 hours or greater, after ingestion of a meal. The meal may be administered at, for example, about 75 minutes, about 90 minutes, about 120 minutes, about 160 minutes, about 240 minutes, or at anytime after the oral administration suitable for demonstrating increased pH about 4.0 with administration of the present composition.

Studies can be conducted to evaluate the bioavailability of a compositions of the present invention using a randomized, balanced, open label, single dose, crossover design. A study, for example, can be performed using 12 healthy male and/or female volunteers between the ages of 18 and 35. Blood samples are removed at 0, 0.5, 1, 2, 3, 4, 6, 8, 10, 12, 15 and 25 hours. Except for the “fed” treatment in which the subjects receive a standard high fat breakfast, no food is allowed until a standard lunch is served four hours after the dose is administered. The data from each time point is used to derive pharmacokinetic parameters, such as, area under plasma concentration-time curve (“AUC”), including AUC₍₀₋ₜ₎, AUC_{(0-inf)}, mean peak plasma concentration (Cₘₐₓ) and time to mean peak plasma concentration (Tₘₐₓ). The data can be used to confirm that the composition of the present invention provides the appropriate release characteristics.

The compositions of the present invention can also be evaluated under a variety of dissolution conditions to determine the effects of pH, media, agitation and apparatus. For example, dissolution tests can be performed using a USP Type II or III (VanKel Bio-Dis II) apparatus. Effects of pH, agitation, polarity, enzymes and bile salts can also be evaluated.

For the same of brevity, all patents and other references cited herein are incorporated by reference in their entirety.

### EXAMPLES

The present invention is further illustrated by the following examples, which should not be construed as limiting in any way. The practice of the present invention will employ, unless otherwise indicated, conventional techniques of pharmacology and pharmaceutics, which are within the skill of the art. The experimental procedures to generate the data shown are discussed in more detail below. The invention has been described in an illustrative manner, and it is to be understood that the terminology used is intended to be in the nature of description rather than of limitation.

### Example 1

### Abbreviations, Standards, and Reagent Sources

This example describes abbreviations, standards, reagent sources, and various pharmacokinetic and pharmacodynamic parameters disclosed herein.

SAN-05/OSB-IR (powder for suspension): Omeprazole (20 mg or 40 mg) with sodium bicarbonate 1680 mg (20 mEq), for immediate-release, reconstituted to a total volume of 20 mL of water at 1 or 2 mg/mL.

SAN-10/OME-IR (capsule): Omeprazole (20 mg or 40 mg) with an antacid complex, for immediate-release. Antacid complexes included: sodium bicarbonate alone; sodium bicarbonate with magnesium hydroxide; and sodium bicarbonate with calcium carbonate.

SAN-15 /OME-IR (chewable tablet): Omeprazole (20 mg or 40 mg) with an antacid complex, for immediate-release. Antacid complexes included: sodium bicarbonate alone; sodium bicarbonate with magnesium hydroxide; and sodium bicarbonate with calcium carbonate.

OME-DR (enteric-coated): Omeprazole (20 mg or 40 mg) with enteric-coating, for delayed-release.

Pharmacokinetic parameters disclosed herein include: (1) parameters obtained directly from the data without interpolation, including plasma omeprazole concentration, peak omeprazole plasma concentration (Cₘₐₓ), and time to peak omeprazole plasma concentration (Tₘₐₓ); (2) terminal elimination rate constant (kₑₗ) determined from a log-linear regression analysis of the terminal plasma omeprazole concentrations; (3) terminal elimination half-life (t_{1/2}) calculated as 0.693/kₑₗ; (4) area under the omeprazole plasma concentration-time curve from time zero to time “t” (AUC₀₋ₜ), calculated using the trapezodial rule with the plasma concentration at time “t” being the last measurable concentration; (5) area under the omeprazole plasma concentration-time curve from time zero to time infinity (AUC_{0-inf}), calculated as AUC₀₋ₜ+Cₜ/kₑₗ, where Cₜ is the last measurable plasma concentration and kₑₗ is the terminal elimination rate constant defined above.

Pharmacodynamic parameters disclosed herein include: (1) mean gastric acid concentration; (2) onset time of gastric pH increase; (3) gastric pH over time; (4) length of time gastric pH is>4; (5) percentage (%) of time gastric pH is time pH>4 (in figures as “% time pH>4”); (6) median gastric pH; and (7) integrated gastric acidity, which is expressed as mM acid×time, (mmol acid×hr/L) is calculated as the cumulative time-weighted average of mean gastric acid concentration, as follows:

Acid concentration (mM)=1000×10-pH

Acidity (mmol.hr/L)=(acid in mM at time “t”+acid in mM at time “t-1”)/2×(t−t-1)

Values for acidity are summed cumulatively

Definitions used for convenience: (1) onset of action, the earliest time that the value with active treatment was significantly different from the corresponding baseline value; (2) duration of action, the latest time that the value with active treatment was significantly different from the corresponding baseline value; (3) magnitude of effect, maximum value at a given post-dosing interval.

Meals

Standardized breakfast: 2 large fried eggs, 2 strips of bacon, 2 slices toast/white bread, 10 grams butter, 4 ounces hash brown potato, 1 cup whole milk, and 6 fluid ounces chilled orange juice. Standardized high fat lunch: 240 grams potatoes (chips), fine cut, frozen, fried in blended oil; 225 grams cod, in batter, fried in blended oil; 70 grams peas, frozen, boiled in salt water; 120 grams custard, made with whole milk; 110 grams sponge pudding, with jam; and 200 ml whole milk.

Reagents

Chewable antacid tablets (Murty Pharmaceuticals, Inc., Lexington, Ky.) contained 1260 mg NaHCO₃ and 750 mg CaCO₃, as well as common excipients. USP grade bulk omeprazole was obtained from commercial sources.

In some experiments, Omeprazole powder was mixed with powdered peppermint flavoring and Equal® Sweetener before administration.

Prilosec® capsules containing enteric-coated omeprazole granules (40 mg) and Nexium®D capsules containing enteric-coated esomeprazole granules (40 mg) are marketed by AstraZenecae.

Abbrviations

Acitrel®: 20 mg omeprazole, powder for suspension, OSB-IR formulation

AE: Adverse event

ALT: (SGPT) Alanine aminotransferase

AST: (SGOT) Aspartate aminotransferase

AUC_{(0.inf)}: Area under the plasma drug concentration curve calculated from 0 time extrapolated to infinity

AUC₍₀₋ₜ₎: Area under the plasma drug concentration curve calculated from 0 time to last time point evaluated

BUN: Blood urea nitrogen

Cₘₐₓ: Peak plasma concentration of drug being measured

Cₜ: Plasma concentration at a given time

H2: Histamine H2 receptor

Kₑₗ: Elimination rate constant

LC-MS: Liquid chromatography—mass spectoscopy

NaHCO₃: Sodium bicarbonate

OSB-IR PWD F/S: Omeprazole sodium bicarbonate, immediate-release, powder for suspension

PK: Pharmacokinetic

PPI: Proton pump inhibitor

qAM: Every morning

Rapinex®: SAN-15 chewable tablet formulation

SAS: Statistical analysis software

SOS: Simplified omeprazole solution/suspension

Tₘₐₓ: Time at which Cₘₐₓ is observed

T_{1/2}: Half life of drug elimination

Pharmacokinetic and Pharmacodynamic Measurements

Blood samples (10 mL) were taken within 30 minutes predose and up to 12 hours postdose; eg, postdose at 5, 10, 15, 30, 45, 60, 90, 120, 180, 240, 300, 360 minutes, and up to 12 hours in some studies. Baseline gastric pH data were collected for each subject at a screening visit prior to the testing periods. Baseline data were collected using an ambulatory, single disposable probe and pH recording system. The electrode was calibrated at 37° C. using standard polyelectrolyte solutions at pH 1.07 and pH 7.01. The location of the subject's lower esophageal sphincter (LES) was located manometrically and the distance from the lower border of the nares to the upper border of the LES was be recorded.

### Example 2

### Trial Protocols

This example describes several trial protocols used to obtain results described herein.

SAN-15-C01 Trial Protocol

This trial protocol is designed as a single-dose crossover study, wherein each subject received one or two chewable antacid tablets administered concomitantly with omeprazole powder during each treatment period, for up to six treatment periods. Each period was followed by a 7-14 day washout. The same treatment was administered to all subjects in each trial period:

Period 1: One (1) antacid tablet (formulation 1:3) plus 40 mg omeprazole powder administered in the fasted state.

Period 2: 20 mEq sodium bicarbonate plus 40 mg omeprazole powder as an aqueous suspension administered in the fasted state.

Period 3: Prilosec 40 mg delayed-release capsule administered in the fasted state.

Period 4: One (1) antacid tablet (formuation 1:3) plus 40 mg omeprazole powder administered 1 hour after initiating a meal.

Period 5: One (1) antacid tablet (formulation 1:1) plus 40 mg omeprazole powder administered in the fasted state.

Period 6: Two (2) antacid tablets (formulation 1:1) plus 40 mg omeprazole powder administered 1 hour after initiating a meal.

For the periods including omeprazole powder plus tablet administration, the subject received omeprazole powder administered directly onto the dorsal mid-tongue. Immediately thereafter, subjects were given one chewable antacid tablet, which they began chewing. The subject continued to chew the tablet while mixing it with omeprazole powder, and carefully avoided swallowing the powder immediately. One minute after initiating chewing (and after completely swallowing trial medications), each subject drank 120 mL of water, swishing the oral contents before swallowing.

Gastric pH was monitored continuously for up to 6 hours after each dose of a given treatment, and blood samples were obtained for determination of plasma omeprazole concentrations, on control and active treatment days. Pharmacodynamic evaluations may include include measurements of integrated gastric acidity; mean pH; and the % time pH>3, % time pH>4, and % time pH>5. Pharmacokinetic evaluations included plasma omeprazole concentration at each sampling time; and plasma omeprazole Cₘₐₓ, Tₘₐₓ, kₑₗ, AUC₍₀₋ₜ₎ and AUC_{(0-inf)}.

This trial assessed the pharmacokinetics and gastric acidity of omeprazole/antacid as an immediate-release formulation of omeprazole.

SAN-15-C01B Trial Protocol

This trial protocol was designed as a single-dose crossover study, and each subject received an oral antacid formulation with an omeprazole/antacid formulation, omeprazole powder alone, or Prilosec in each period, for six treatment periods. Each period was followed by a 7-21 day washout. The same treatment was administered to all subjects in each trial period:

Period 1: One antacid tablet (30 mEq of a 1:1 formulation of sodium bicarbonate and calcium carbonate) plus 40 mg omeprazole powder administered 1 hour prior to ingestion of standardized breakfast.

Period 2: One antacid tablet (30 mEq of a 1:1 formulation of sodium bicarbonate and calcium carbonate) plus 40 mg omeprazole powder administered 30 minutes prior to ingestion of standardized breakfast.

Period 3: One antacid tablet (30 mEq of a 1:1 formulation of sodium bicarbonate and calcium carbonate) plus 40 mg omeprazole powder administered 3 hours after initiating ingestion of standardized breakfast.

Period 4: One Nexium™ tablet (40 mg esomeprazole) administered 30 minutes prior to ingestion of a standard breakfast

Period 5: One antacid tablet (30 mEq of a 1:1 formulation of sodium bicarbonate and calcium carbonate) plus 80 mg omeprazole powder administered 4 hours after initiating ingestion of a standard breakfast.

Period 6: One Prilosec® 40 mg capsule administered 30 minutes prior to ingestion of a standard breakfast.

For the periods including omeprazole powder plus tablet administration, the subject received omeprazole powder administered directly onto the dorsal mid-tongue. Immediately thereafter, subjects were given one chewable antacid tablet, which they began chewing. The subject continued to chew the tablet while mixing it with omeprazole powder, and carefully avoided swallowing the powder immediately. One minute after initiating chewing (and after completely swallowing trial medications), each subject drank 120 mL of water, swishing the oral contents before swallowing.

For periods requiring a meal, subjects fasted for at least 10 hours overnight and were allowed water ad libitum until 2 hours prior to administration. The standardized breakfast was eaten within 30 minutes. For Period 1, 120 mL water was also given at 1 hour prior to initiating ingestion of the meal. For Period 2, 120 mL water was also given at one half hour prior to initiating the meal. For 6 hours after each dose of a given treatment, gastric pH was monitored and blood samples obtained for determination of plasma omeprazole concentration.

Pharmacodynamic evaluations may include measurements of gastric pH over time; onset time of gastric pH increase; and the extent and duration of pH increase (above pH 3 or pH 4). Pharmacokinetic evaluations included plasma omeprazole concentration at each sampling time; and plasma omeprazole Cₘₐₓ, Tₘₐₓ, kₑₗ, AUC₍₀₋ₜ₎ and AUC_{(0-inf)}.

SAN-15 is a chewable antacid tablet of omeprazole that provides more rapid pH control and relief of gastric symptoms than currently marketed proton pump inhibitors. In this formulation, omeprazole is protected by a mixture of antacids, thereby limiting exposure of omeprazole to gastric acid.

The Cₘₐₓ of omeprazole is higher and occurs sooner after the first dose than after the first dose of Prilosec. This allows the omeprazole and antacid formulation to be administered in close proximity to meals that often induce or are associated with gastric acid-related symptoms. This trial assessed pharmacokinetics and gastric acidity under these conditions, indicating that omeprazole plus antacid combination may be useful for treating meal-induced or meal-associated heartburn.

SAN-15-C01C Trial Protocol

This trial protocol is designed as a single-dose crossover trial. Each healthy volunteer subject received an oral antacid formulation with omeprazole; omeprazole powder alone; Prilosec capsule (US formulation); and Nexium capsule (US formulation) in each period. Each dose was followed by a 7-14 day washout. The same treatment was administered to all subjects in each trial period:

Period 1: A single 80 mg oral dose of omeprazole powder administered with one chewable antacid tablet (1260 mg NaHCO₃ and 750 mg CaCO₃) administered 90 minutes after a standardized breakfast.

Period 2: A single 40 mg oral dose of omeprazole powder administered in the fasted state.

Period 3: A single 40 mg oral dose of omeprazole powder administered with one chewable antacid tablet (1260 mg NaHCO₃ and 750 mg CaCO₃) administered 90 minutes after a standardized breakfast.

Period 4: A single 40 mg oral dose of one Nexium™ capsule (esomeprazole, US formulation) administered 90 minutes after a standardized breakfast.

Period 5: A single 40 mg oral dose of omeprazole powder administered 90 minutes after a standardized breakfast.

Period 6: A single 120 mg oral dose of omeprazole powder administered with one chewable tablet (1260 mg NaHCO₃ and 750 mg CaCO₃) administered 90 minutes after a standardized breakfast.

For the periods including omeprazole powder plus tablet administration, the subject received omeprazole powder administered directly onto the dorsal mid-tongue. Immediately thereafter, subjects were given one chewable antacid tablet, which they began chewing. The subject continued to chew the tablet while mixing it with omeprazole powder, and carefully avoided swallowing the powder immediately. One minute after initiating chewing (and after completely swallowing trial medications), each subject drank 120 mL of water, swishing the oral contents before swallowing.

For periods requiring a meal, subjects fasted for at least 10 hours and were allowed water ad libitum until 2 hours prior to administration. Gastric pH monitoring was recorded for up to 11 hours beginning at time zero. The standard breakfast was ingested over 30 minutes beginning 90 minutes after the initiation of pH monitoring.

For periods including dosing after a meal, subjects fasted for at least 10 hours. On Day 0, ninety minutes of probe pH monitoring was started prior to initiating ingestion of the standardized breakfast, which was eaten within 30 minutes. The pH monitoring continued for 9.5 hours after initiating ingestion of breakfast. For Periods 1 and 2, and one subsequent period, 120 mL of water only was administered 90 minutes after initiating ingestion of the standard breakfast. On Day 1, after fasting overnight for at least 10 hours, 90 minutes of probe pH monitoring was started prior to initiating ingestion of the standardized breakfast, which was eaten within 30 minutes. The pH monitoring continued for 9.5 hours after initiating ingestion of breakfast. Trial medications were administered 90 minutes after initiating ingestion of the standardized breakfast.

Pharmacokinetic evaluations include plasma omeprazole and esomeprazole concentration over time; and plasma omeprazole and esomeprazole Cₘₐₓ, Tₘₐₓ, kₑₗ, T_{1/2}, AUC₍₀₋ₜ₎, and AUC_{(0-inf)}. Pharmacodynamic evaluation can include onset time of gastric pH increase, gastric pH over time, and % time pH>4.

The Cₘₐₓ of omeprazole is higher and occurs sooner after the first dose with antacid than after the first dose of Prilosec or Nexium. The omeprazole/antacid formulations can be administered in close proximity to meals that are often associated with acid-related symptoms thereby treating, for example, meal-induced or meal associated heartburn. The SAN-15-CO1C trial assessed pharmacokinetics and gastric pH under these conditions.

SAN-15-C01D Trial

This trial is an open-label, single-dose, crossover trial, and each subject received up to ten different oral omeprazole formulations, one in each of ten treatment periods. Each dose was followed by at least a 7 day washout. Omeprazole (40 mg) was administered with up to 1680 mg sodium bicarbonate and/or up to 600 mg magnesium hydroxide and/or up to 750 mg calcium carbonate. SAN-15 (Patheon Pharmaceuticals Inc., Cincinnati, Ohio) formulations contained ≦40 mEq antacid(s) plus 40 mg omeprazole (with or without incorporation into a chewable tablet), and SAN-10 (Pharm Ops Inc., Phillipsburg, New Jersey) capsules contained ≦40 mEq antacid(s) and 40 mg Omeprazole. All formulations were administered with 120 mL of water after an overnight fast and 1 hour prior to a standardized high-fat breakfast. Within a given treatment period, the same treatment was administered to all subjects.

Omeprazole was delivered either as Prilosec or as an immediate-release formulation (without an enteric-coating). It was formulated as uncoated or microencapsulated granules in a loose powder, as powder in a capsule, in a chewable tablet, or in a swallowable tablet. The antacid was administered concomitantly as antacid tablets, or the omeprazole and antacid were combined in a tablet or capsule. Pharmacokinetic evaluations were as previously described.

When omeprazole powder plus tablet was administered, the subject received omeprazole powder administered directly onto the dorsal mid-tongue. Immediately thereafter, subjects were given one chewable antacid tablet, which they began chewing. The subject continued to chew the tablet while mixing it with omeprazole powder, and carefully avoided swallowing the powder immediately. One minute after initiating chewing (and after completely swallowing trial medications), each subject drank 120 mL of water, swishing the oral contents before swallowing.

Administering omeprazole plus antacid formulations in close proximity to meals that are often associated with acid-related symptoms may be useful for treating, for example, meal-induced heartburn.

OSB-IR-C02 and OSB-IR-C06 Trial Protocols

Both trials are randomized crossover trials, where each healthy subject received seven consecutive daily doses of either Prilosec® 40 mg or OSB-IR 40 mg (OSB-IR-CO2) or Prilosec® 20 mg or OSB-IR 20 mg (OSB-IR-CO6) administered qAM one hour prior to initiating ingestion of a standardized breakfast: for Period 1,; an eighth dose of OSB-IR (20 or 40 mg) was administered at the completion of a standardized meal on Day 8 for those subjects who received OSB-IR in Period 1. A 10-14 day washout occurred prior to the beginning of Period 2. The alternative dosage form was then administered once daily for seven days (Period 2).

Period 1: 40 mg or 20 mg omeprazole (OSB-IR-CO2 or OSB-IR-CO6, respectively) as either OSB-IR or Prilosec administered for seven consecutive single daily doses, fasting; (plus Dose 8 with meal only for subjects who received OSB-IR). Twelve (12) hour pharmacokinetics and 24 hour pH monitored after Doses 1 and 7;12-hr PK monitored after Dose 8.

Period 2: 40 mg or 20 mg omeprazole (the alternative formulation to that used in Period 1) (OSB-IR-CO2 or OSB-IR-CO6, respectively) for seven consecutive single daily doses; fasting. Twelve (12) hour pharmacokinetics and 24 hour pH monitored after Doses 1 and 7.

For both OSB-IR-CO2 and OSB-IR-CO6 trials, baseline gastric pH was recorded before dosing on Day 1 of Periods 1 and 2. For 24 hr after each dose of a given treatment on Days 1 (Dose 1) and 7 (Dose 7) of each period, gastric pH was monitored and blood samples obtained for determination of plasma omeprazole. Doses 2 to 6 were administered after an overnight fast with water allowed ad libitum. One hour postdose, subjects were allowed to consume food and non-alcoholic beverages ad libitum. Subjects who received OSB-IR in Period 1 only continued for Dose 8 of OSB-IR on Day 8 administered after the 24-hr monitoring period after Dose 7 and at completion of a standardized breakfast. After the washout period, the procedures outlined above for Period 1 (except no Dose 8) were repeated for the alternative dosage form (Period 2).

For the OSB-IR-CO6 trial, subjects who received OSB-IR in Period 2 only continued for Dose 8 of OSB-IR on Day 8 administered after completion of the 24-hour monitoring period after Dose 7 and one hour before beginning a standardized breakfast on Day 8. These subjects consumed standardized meals at 1300 and 1800 hours after Dose 8 and did not consume any additional food on Day 8. At 2200 hours, subjects took another OSB-IR 20 mg dose (Dose 9). These subjects were pH monitored for 24 hours after Dose 8 continuously.

Pharmacokinetic evaluations can include plasma omeprazole concentration over time; and plasma omeprazole Cₘₐₓ, Tₘₐₓ, k_{cl}, T_{1/2}, AUC₍₀₋ₜ₎, and AUC_{(0-inf)}. Pharmacodynamic evaluation can include integrated gastric acidity, mean acid concentration, time gastric pH>4, time gastric pH<4 and median gastric pH.

OSB-IR permits delivery of omeprazole as a suspension, wherein the omeprazole is protected from gastric acid by the sodium bicarbonate contained in the formulation. A liquid form of omeprazole makes the drug available to patients for whom a solid dosage form is unsatisfactory, for example, the very young, the elderly, the neurologically impaired, and those with nasogastric (NG) tubes.

The bioavailability (AUC) and pharmacodynamics (gastric acid suppression) of OSB-IR and Prilosec were assessed and found to be equivalent at steady state. These trials also determined the effect of food on pharmacokinetics of OSB-IR. This OSB-IR-CO6 trial further revealed that omeprazole plus antacid formulation administered before bedtime is useful for reducing nocturnal gastric acidity and therefore potentially for heartburn.

OSB-IR-C05

This trial is designed as a single-period, open-label design. Two 40 mg doses of omeprazole sodium bicarbonate immediate-release suspension (OSB-IR) were administered to healthy subjects under fasting conditions on the first day of therapy, with a between-dose interval of six hours. Blood samples were collected over a total of 18 hr.

Omeprazole delivered as the liquid dosage form (OSB-IR suspended in water prior to administration) was protected from gastric acid by sodium bicarbonate contained in the formulation.

OSB0-IR-C03 Trial

This was a comparision of Omeprazole plus sodium bicarbonate immediate-release oral suspension to intravenous cimetidine for the prevention of upper gastrointestina bleeding in critically ill patients.

OSB-IR suspension (40 mg omeprazole plus 1680 mg sodium bicarbonate) was administered to half the patients and cimetidine (300 mg bolus, followed by 50 mg/hr) was administered to the other half. Gastric aspirates were assessed for bleeding and pH. Clinically significant bleeding was bright red blood for 5-10 min on Days 1-14, or Gastroccult positive coffee ground material for 8 consecutive hours on days 1-2, or 2-4 hrs on days 3-14 (after enteral feeding began). 359 critically ill patients were treated.

Administering omeprazole plus antacid formulations to patients having upper GI bleeding or at risk of developing upper GI (UGI) bleeding can be useful for preventing bleeding, as well as reducing or preventing associated complications (e.g., death).

### Example 3

### Omeprazole is Well Absorbed and Rapidly Absorbed in the Presence of Antacid

This example describes results indicating that omeprazole is well absorbed in the presence of antacid, and that a single oral dose of omeprazole antacid complex is rapidly absorbed (see example 8 for the effects of omeprazole antacid complex on gastric acidity). To compare the pharmacokinetic characteristics of omeprazole plus antacid-immediate release to those of omeprazole alone, studies were performed as described in the OSB-IR-CO1C trial protocol.

The pharmacokinetic profiles of omeprazole powder plus chewable antacid tablets, omeprazole powder alone, Prilosec® capsules (omeprazole), and Nexium® capsules (esomeprazole magnesium) in the context of different dosing regimens relative to the ingestion of meals were performed as described in the SAN-15-CO1C trial protocol. These results from trial SAN-15-CO1C, summarized in Table 3.A).

TABLE 3.A
Pharmacokinetics of Omeprazole Powder (40 mg)
Administered With or Without Antacid (Pre-meal)

| | Number of | Cₘₐₓ ng/mL | AUC₍₀₋ₜ₎ng × hr/mL |
|---|---|---|---|
| | Subjects | (Median) | (Median) |
| Control | 10 | — | — |
| Omeprazole Powder | 10 | 186.4 | 225 |
| Administered 1 hour | | | |
| Pre-meal | | | |
| Omeprazole Powder | 10 | 911.5 | 965.7 |
| Plus 30 mEq | | | |
| Antacid Administered | | | |
| 1 hour Pre-meal | | | |

Median AUC_{(0-inf)} for omeprazole from omeprazole antacid complex-immediate release, 966 ng.hr/mL, was significantly higher (P=0.0355) than that from omeprazole alone, AUC_{(0-inf)} 225 ng.hr/mL. These results indicate that omeprazole without concomitant antacid is weakly absorbed (low bioavailability).

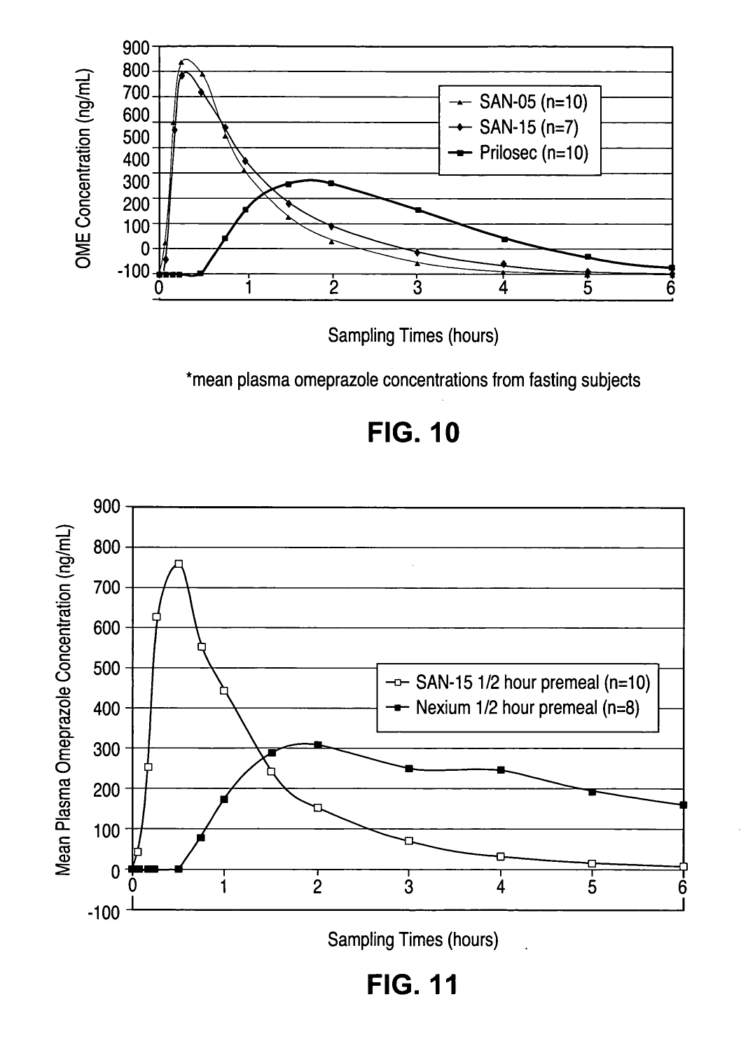

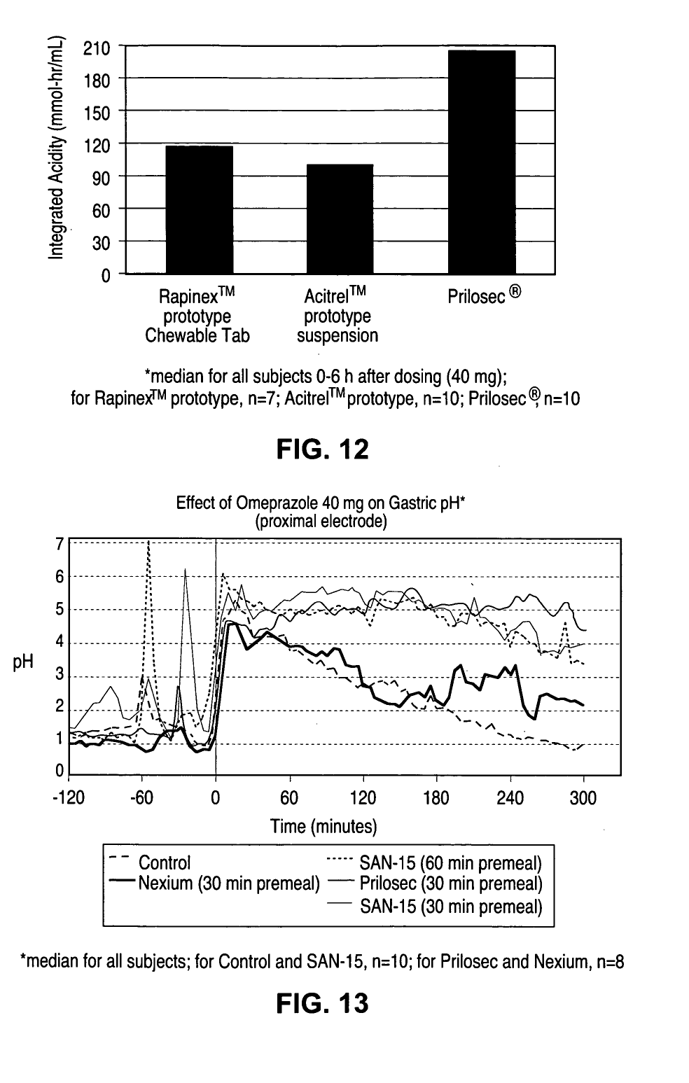

The pharmacokinetic results of the study illustrated in FIG. 10 indicate that when administered to fasting subjects, omeprazole powder with antacid (either as a suspension or as a chewable antacid tablet) is more rapidly absorbed than omeprazole delivered as delayed-release (enteric-coated) Prilosec®

FIG. 11 indicates that a single pre-meal dose of 40 mg of omeprazole powder plus 30 mEq antacid given 30 minutes before a meal is more rapidly absorbed than Nexium® 40 mg given 30 minutes before a meal.

### Example 4

### Omeprazole Plus Antacid Formulation has More Rapid Absorption and Comparable Bioavailability as Delayed-Release Omeprazole Formulation

This example describes results indicating that omeprazole antacid complex has more rapid absorption and comparable bioavailability as delayed-release omeprazole formulation.

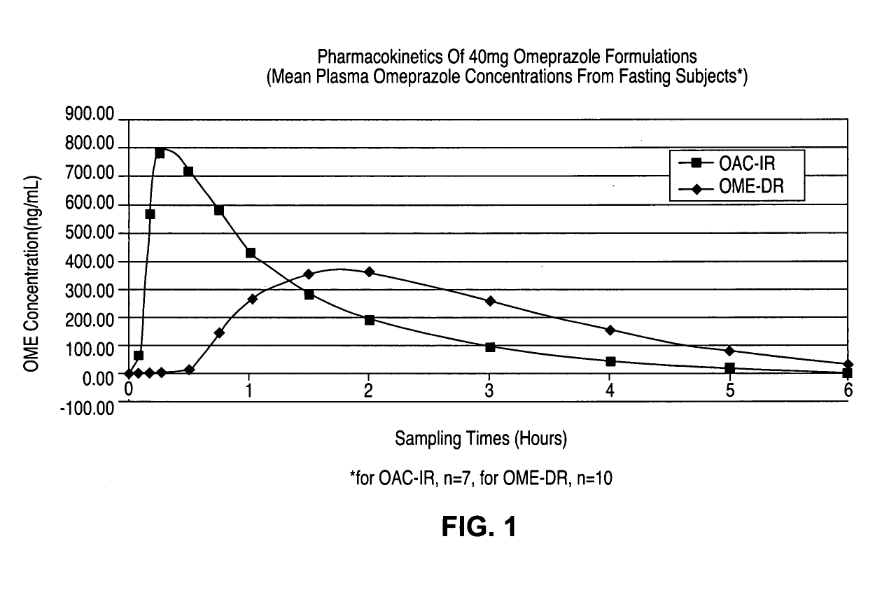

To compare omeprazole antacid complex-immediate release composition to omeprazole enteric-coated granules with regard to PK and gastric pH, a crossover trial was performed in 10 fasting subjects receiving a single capsule of 40mg omeprazole enteric-coated granules (omeprazole delayed-release), and 7 receiving 40 mg omeprazole powder plus a chewable tablet composed of 1260 mg NaHCO₃ and 750 mg CaCO₃ (omeprazole antacid complex-immediate release). Plasma omeprazole concentration was measured over a 6-hour postdose period (FIG. 1) and gastric pH was measured for 1 hour before and 6 hours after dosing.

Omeprazole absorption from OAC-IR was more rapid (T₍ₘₐₓ₎ 25 min; C₍ₘₐₓ₎ 1019 ng/mL) than from the omeprazole delayed-release formulation (T₍ₘₐₓ₎ 127 min; C₍ₘₐₓ₎ 544 ng/mL). Bioavailability of omeprazole antacid complex-immediate release (AUC_{(0-inf)} 1120 ng×hr/mL) and OME-DR (AUC_{(0-inf)} 1170 ng×hr/mL) were similar (P=0.96). Integrated gastric acidity over the 6-hour postdose period was 43% less with omeprazole antacid complex-immediate release than with omeprazole delayed-release (P=0.071; median for all subjects).

When compared to a marketed omeprazole delayed-release formulation, omeprazole antacid complex-immediate release has more rapid absorption, with similar pharmacodynamic effect. Omeprazole antacid complex-immediate release will be effective in relieving existing and recurrent heartburn, with the antacid producing immediate relief and omeprazole preventing recurrence, severity or duration of subsequent episodes.

### Example 5

### Bioavailability of Omeprazole Plus Sodium Bicarbonate as Compared to Prilosec®

This example describes studies indicating that omeprazole/sodium bicarbonate and Prilosec® are bioequivalent after one day and after 7 days of administration as estabilished by FDA requirements.

To compare the pharmacokinetic and pharmacodynamic characteristics of omeprazole/antacid-immediate release to enteric-coated omeprazole, studies were performed as described in the OSB-IR-CO2 and OSB-IR-CO6 trials with omeprazole (40 mg or 20 mg, respectively) plus 1680 mg of sodium bicarbonate administered as an aqueous suspension. Pharmacokinetic parameters can include AUC_{(0-inf)} for the first and seventh doses of each omeprazole formulation, Cₘₐₓ for the first and seventh doses of each omeprazole formulation, and Tₘₐₓ, Kel, T_{1/2}, AUC₍₀₋ₜ₎ for the first and seventh doses of each omeprazole formulation.

The results of omeprazole pharmacokinetic parameters between omeprazole plus sodium bicarbonate administration pre-meal and Prilosec® administration pre-meal are summarized in Tables 5.A., 5.B. and 5.C.

TABLE 5.A
Plasma Omeprazole Concentration
Omeprazole/Sodium Bicarbonate 40 mg vs. Prilosec ® 40 mg (Day 1)

| | Omeprazole/Sodium | | | | | | | |
|---|---|---|---|---|---|---|---|---|
| | Bicarbonate | | | Prilosec ® 40 mg | | | | |
| | 40 mg (Fasting) | | | (Fasting) | | | | |
| | | Arithmetic | | | Arithmetic | | 90% | % Mean |
| Parameters | N | Mean | SD | N | Mean | SD | Cl | Ratio |
| Cₘₐₓ (ng/mL) | 32 | 1412 | 616.2 | 32 | 1040 | 579.1 | — | — |
| Tₘₐₓ (hr) | 32 | 0.44 | 0.19 | 32 | 2.34 | 2.40 | — | — |
| AUC₍₀₋ₜ₎ (ng × hr/mL) | 32 | 2180 | 2254 | 32 | 2460 | 2546 | — | — |
| AUC_{(0-inf)} (ng × hr/mL) | 32 | 2228 | 2379 | 31 | 2658 | 2888 | — | — |
| T_{1/2} (hr) | 32 | 1.00 | 0.63 | 31 | 1.21 | 0.73 | — | — |
| Kel (1/hr) | 32 | 0.89 | 0.38 | 31 | 0.73 | 0.30 | — | — |
| In(Cₘₐₓ) | 32 | 7.15 | 0.47 | 32 | 6.74 | 0.74 | 124.0-184.1 | 151.1 |
| In[AUC₍₀₋ₜ₎] | 32 | 7.34 | 0.80 | 32 | 7.41 | 0.91 | 83.9-103.5 | 93.2 |
| Ln[AUC_{(0-inf)}] | 32 | 7.35 | 0.80 | 31 | 7.48 | 0.87 | 82.4-93.7 | 87.9 |

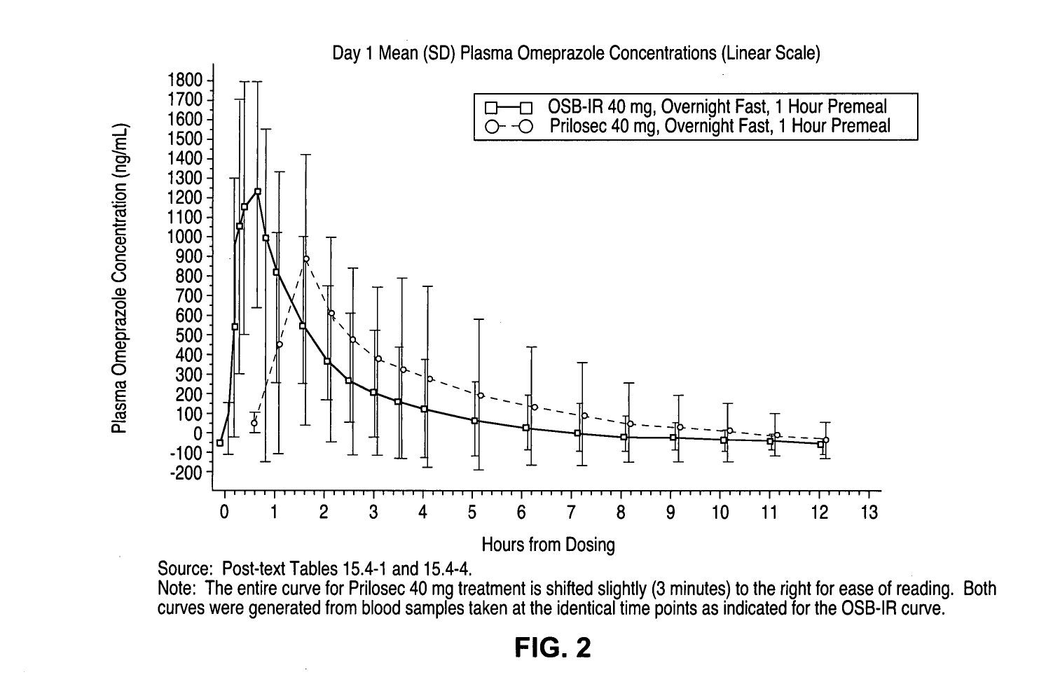

After one dose, 40 mg omeprazole plus 1680 mg sodium bicarbonate and Prilosec® (40 mg) were bioequivalent with respect to AUC (Table 1). The mean ratio for omeprazole plus sodium bicarbonate to Prilosec® was 87.9% for AUC_{(0-inf)} with the boundaries of the 90% CI within 80% and 125% compared with Prilosec®. Mean plasma omeprazole concentrations versus time plot for Day 1 are illustrated in FIG. 2.

TABLE 5.B
Plasma Omeprazole Concentration
Omeprazole/Sodium Bicarbonate 40 mg vs. Prilosec ® 40 mg (Day 7)

| | Omeprazole/Sodium | | | | | | | |
|---|---|---|---|---|---|---|---|---|
| | Bicarbonate | | | Prilosec ® 40 | | | | |
| | 40 mg (Fasting) | | | mg (Fasting) | | | | |
| | | Arithmetic | | | Arithmetic | | | % Mean |
| Parameters | N | Mean | SD | N | Mean | SD | 90% Cl | Ratio |
| Cₘₐₓ | 31 | 1954 | 654.0 | 31 | 1677 | 645.5 | — | — |
| (ng/mL) | | | | | | | | |
| Tₘₐₓ (hr) | 31 | 0.58 | 0.23 | 31 | 1.77 | 0.90 | — | — |
| AUC₍₀₋ₜ₎ (ng × hr/mL) | 31 | 4555 | 2586 | 31 | 4506 | 2522 | — | — |
| AUC_{(0-inf)} | 31 | 4640 | 2741 | 31 | 4591 | 2640 | — | — |
| (ng × hr/mL) | | | | | | | | |
| Ln(Cₘₐₓ) | 31 | 7.51 | 0.40 | 31 | 7.34 | 0.43 | 107.2-133.2 | 119.5 |
| Ln[AUC₍₀₋ₜ₎] | 31 | 8.26 | 0.63 | 31 | 8.25 | 0.62 | 95.4-109.1 | 102.0 |
| Ln[AUC_{(0-inf)}] | 31 | 8.27 | 0.63 | 31 | 8.26 | 0.63 | 95.3-109.0 | 101.9 |

TABLE 5.C
Plasma Omeprazole Concentration
Omeprazole/Sodium Bicarbonate 20 mg vs. Prilosec ® 20 mg (Day 7)

| | Omeprazole/Sodium | | | | | | | |
|---|---|---|---|---|---|---|---|---|
| | Bicarbonate 40 mg | | | Prilosec ® 40 mg | | | | |
| | (Fasting) | | | (Fasting) | | | | |
| | | Arithmetic | | | Arithmetic | | 90% | % Mean |
| Parameters | N | Mean | SD | N | Mean | SD | Cl | Ratio |
| Cₘₐₓ | 31 | 902 | | 31 | 573 | | — | — |
| (ng/mL) | | | | | | | | |
| AUC_{(0-inf)} | 31 | 1446 | | 31 | 1351 | | — | — |
| (ng × hr/mL) | | | | | | | | |
| In(Cₘₐₓ) | | | | | | | 142-174 | 157 |
| Ln[AUC_{(0-inf)}] | | | | | | | 100-114 | 107 |

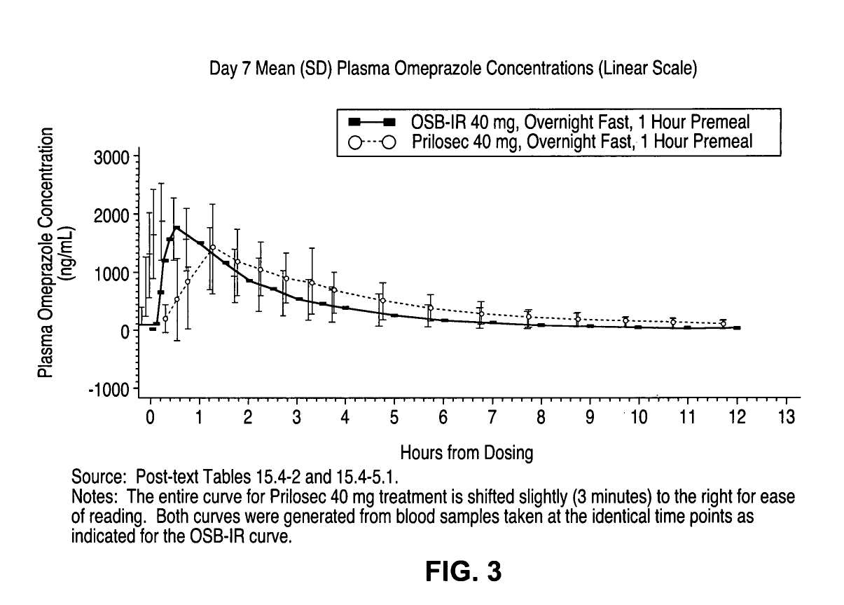

The primary bioequivalence endpoint was AUC₍₀₋ᵢₙₜ₎ at steady state (Day 7). The 40 mg of omeprazole plus 1680 mg of sodium bicarbonate and the 40 mg of Prilosec® administered once a day in the morning were bioequivalent (Table 2a). The AUC_{(0-inf)} mean ratio was 101.9% with a 90% confidence interval (CI) of 95.3% to 109.0%. The Cₘₐₓ for the omeprazole plus sodium bicarbonate solution at steady state was slightly higher than for Prilosec® with a mean ratio of 119.5% and 90% CI of 107.2% to 133.2%. Mean plasma omeprazole concentrations versus time for Day 7 are illustrated in FIG. 3.

The mean Tₘₐₓ for Prilosec® tended to decrease over time (2.34 hours for Day 1 versus 1.77 hours for Day 7). The mean Tₘₐₓ for omeprazole plus sodium bicarbonate did not change significantly over time (0.44 hours for Day 1 versus 0.58 hours for Day 7). The mean half-life values were similar for omeprazole plus sodium bicarbonate and Prilosec® (1.0 hours and 1.2 hours, respectively) for Day 1.

### Example 6

### Omeprazole Plus Sodium Bicarbonate is Pharmacodynamically Equivalent to Prilosec®

This example describes results indicating that omeprazole plus sodium bicarbonate and Prilosec® were pharmacodynamically equivalent with respect to steady state 24-hour suppression of integrated gastric acidity. The studies also indicate that omeprazole plus sodium bicarbonate and Priloseco are equally effective in suppressing production of gastric acid, but that the omeprazole plus sodium bicarbonate formulation provides a rapid increase in gastric pH as compared to Prilosec®.

The studies were performed as described in the OSB-IR-CO2 and OSB-IR-CO6 trial protocols. After the drug was administered, gastric pH levels were measured for 24 hours after the administration of the study treatment to the subjects on Days 1 and 7. The primary analysis focused on Day 7 of dosing since the pharmacodynamic effects are maximal by the seventh day of consecutive daily dosing (steady state).

The pharmacodynamic profiles of both omeprazole plus sodium bicarbonate and Prilosec® were assessed as previously described. Integrated gastric acidity was selected as the primary pharmacodynamic parameter for bioequivalence, because it is equally sensitive to change over the entire range of values obtained. In contrast, median gastric pH and the time gastric pH was ≦4 have lower sensitivity in detecting drug-induced change from baseline in gastric acidity.

Differences in the pharmacodynamic effects measured by integrated gastric acidity and the time gastric pH≦4 were assessed using an ANOVA model. Pharmacodynamic equivalence, regarding these parameters, was declared if the upper and lower bounds of the 90% confidence intervals for the ratio of omeprazole plus sodium bicarbonate to Prilosec® were within 80% to 125%. Pharmacodynamic data for omeprazole plus sodium bicarbonate administration pre-meal and Prilosec® administration pre-meal are summarized in Table 6.A.

TABLE 6.A
Assessment of Pharmacodynamic Equivalence Between Omeprazole plus
Sodium Bicarbonate and Prilosec (ANOVA)

| Percent | 40 mg Omeprazole | | | | | | | |
|---|---|---|---|---|---|---|---|---|
| Decrease from | plus 1680 mg sodium | | | | | | | |
| Baseline in 24- | bicarbonate | | | Prilosec ® (40 mg) | | | | |
| Hour Integrated | | Arithmetic | | | Arithmetic | | | % Mean |
| Gastric Acidity | N | Mean | SD | N | Mean | SD | 90% Cl | Ratio |
| Day 1 | 24 | 62.34 | 34.84 | 24 | 61.79 | 39.22 | 85.56-115.38 | 99.36 |
| Day 7 | 24 | 83.33 | 17.07 | 24 | 85.11 | 19.74 | 87.35-118.49 | 101.74 |

Omeprazole plus sodium bicarbonate was pharmacodynamically equivalent to Prilosec® at steady state (Day 7) with respect to the percent decrease from baseline in integrated gastric acidity (Table 3). The boundaries of the 90% CIs were between 80% and 125%.

As depicted in Table 6.B., on Day 1, omeprazole plus sodium bicarbonate and Prilosec® decreased integrated gastric acidity by 70% and 76%, respectively. With increased bioavailability of omeprazole on Day 7, the corresponding decreases were 84% and 93%. The median of the by subject ratios (omeprazole plus sodium bicarbonate/Prilosec®) of the decrease from baseline of integrated gastric acidity was 100%.

TABLE 6.B
Integrated Gastric Acidity with Omeprazole plus
Sodium Bicarbonate and Prilosec ®

| | Integrated Gastric Acidity (mmol × hr/L) | | Omeprazole plus sodium |
|---|---|---|---|
| | 40 mg omeprazole | | bicarbonate/Prilosec ® (%) |
| | plus 1680 mg sodium | | Median of By-Subject |
| Assessment | bicarbonate | Prilosec ® (40 mg) | Ratios |
| Baseline | 2194 | 2061 | — |
| | (1421-2943) | (1358-2763) | |
| Day 1 | 557 | 538 | — |
| | (202-1218) | (169-1262) | |
| Day 7 | 319 | 145 | — |
| | (26-512) | (21-558) | |

Percent Decrease from Baseline to:

| | | | |
|---|---|---|---|
| Day 1 | 70 | 76 | 98 |
| | (52-89) | (46-90) | (83-104) |
| Day 7 | 84 | 93 | 100 |
| | (74-99) | (74-99) | (91-105) |

As illustrated by the wide interquartile ranges both at baseline and after treatment with omeprazole plus sodium bicarbonate and Prilosec®, there was substantial inter-subject variation in the integrated gastric acidity. This degree of variation is characteristic of gastric acid secretion before and after treatment.

AUC_{(0-inf)} and percent decrease from baseline in integrated gastric acidity for omeprazole plus sodium bicarbonate were bioequivalent to Prilosec® on Days 1 and 7 indicated the two treatments were not bioequivalent with regard to Cₘₐₓ, with the upper boundary of the confidence interval around the mean ratio slightly above the defined upper boundary for bioequivalence at steady state. The difference in Cₘₐₓ had no apparent effect on the pharmacodynamics of the omeprazole plus sodium bicarbonate solution.

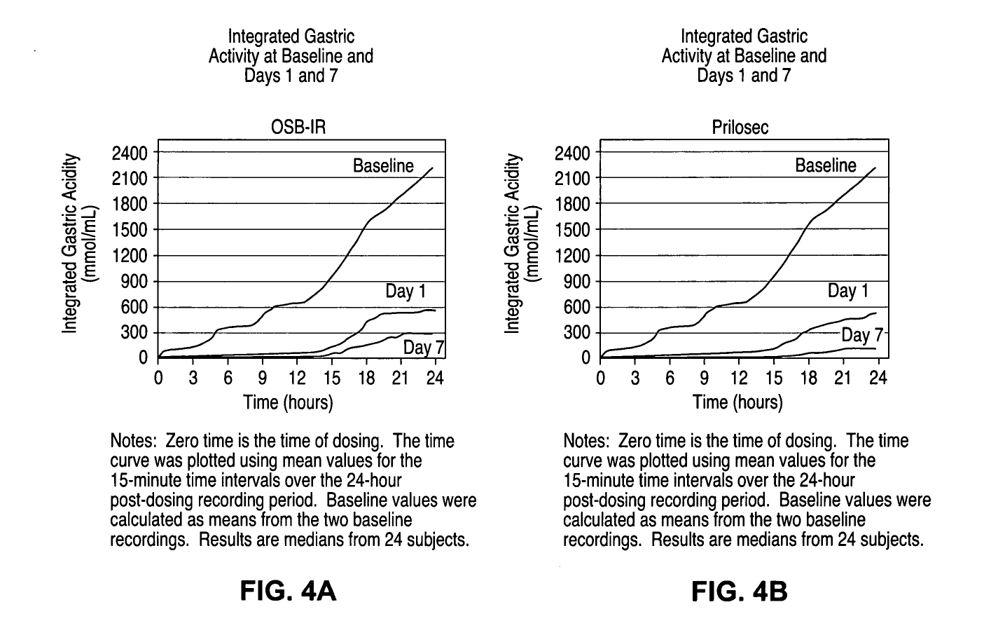

During the baseline period, the integrated gastric acidity increased at a slower rate when meals were ingested (Hours 0 to 12) than during fasting (Hours 13 to 24). FIG. 4a illustrates the effect of 40 mg omeprazole plus 1680 mg sodium bicarbonate on Days 1 and 7 following 3 meals provided during Hours 0 to 12. FIG. 4 also illustrates that on both Days 1 and 7, the configuration of the time-course for integrated gastric acidity with omeprazole plus sodium bicarbonate was similar to that with Prilosec® (FIG. 4b). In particular, both treatments decreased gastric acidity to near zero during the initial 15 hours of the 24 hour recording period.

The values for mean gastric acid concentration are equivalent to the 24-hour integrated gastric acidity divided by 24 and are shown in Table 6.C.

TABLE 6.C
Mean Gastric Acid Concentration with Omeprazole
plus Sodium Bicarbonate and Prilosec ®

| | Mean Gastric Acid Concentration (mM) | |
|---|---|---|
| | 40 mg omeprazole plus 1680 mg | |
| Assessment | sodium bicarbonate | Prilosec ® (40 mg) |
| Baseline | 92 | 86 |
| | (59-123) | (57-115) |
| Day 1 | 24 | 23 |
| | (9-51) | (8-53) |
| Day 7 | 13 | 6 |
| | (1-22) | (1-24) |

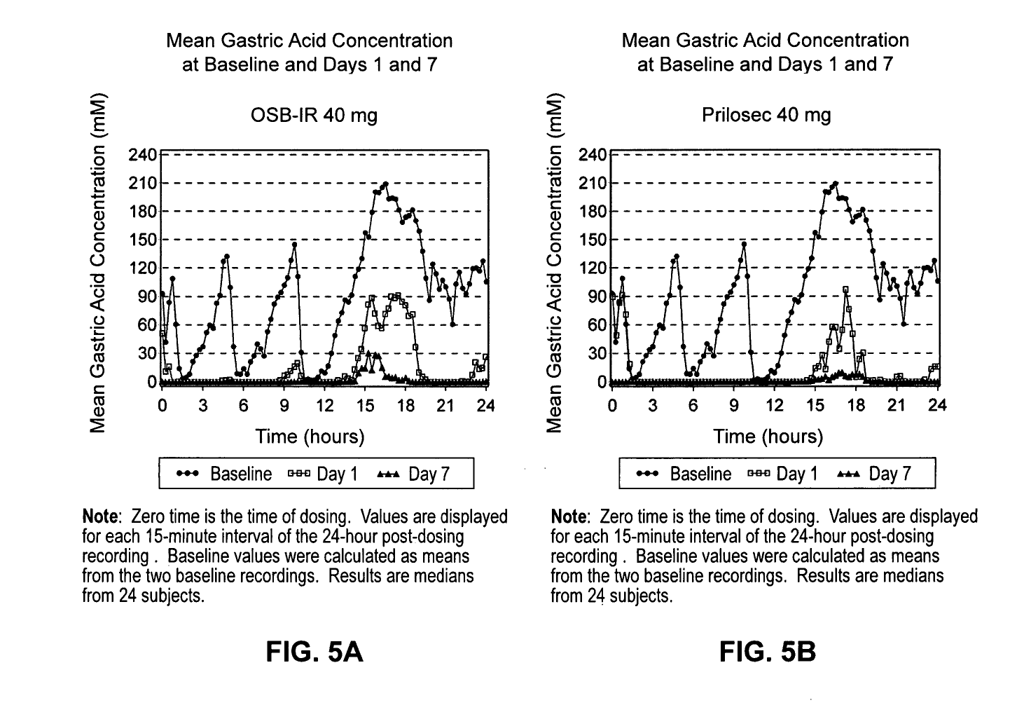

FIG. 5 illustrates the phasic changes in baseline and Days 1 and 7 gastric acid concentration produced by ingestion of meals. At Hours 1, 5, and 10, the baseline acid concentration decreased because the meal neutralized gastric acid. This decrease was then followed by an increase in gastric acid concentration produced, in part, by meal-stimulated gastric acid secretion. At Hour 16, there was a characteristic, but unexplained, increase in the baseline acid concentration.

On Days 1 and 7, omeprazole plus sodium bicarbonate and Prilosec® decreased the gastric acid concentration to near zero during the daytime period from Hours 0 to 14 (FIG. 5). With each treatment, however, there was a nocturnal increase in the acid concentration from Hours 14 to 19 and the magnitude of this increase was lower on Day 7 than on Day 1. Median gastric pH is shown in Table 6.D.

TABLE 6.D
Mean Gastric pH with Omeprazole
plus Sodium Bicarbonate and Prilosec ®

| | Mean Gastric pH (Interquartile Ranges) | |
|---|---|---|
| | 40 mg omeprazole plus 1680 mg | |
| Assessment | sodium bicarbonate | Prilosec ® (40 mg) |
| Baseline | 1.10 | 1.16 |
| | (0.96-1.42) | (1.01-1.51) |
| Day 1 | 3.86 | 4.33 |
| | (2.20-5.39) | (2.81-5.21) |
| Day 7 | 5.20 | 5.20 |
| | (4.14-5.49) | (4.84-5.59) |

Table 6.D. illustrates that a substantial increase in gastric pH from baseline occurred on Days 1 and 7 for both treatments. For both treatments, an increase from baseline of more than 3 pH units on Day 7 was observed that represents a median decrease in gastric acid concentration of greater than 99.9%.

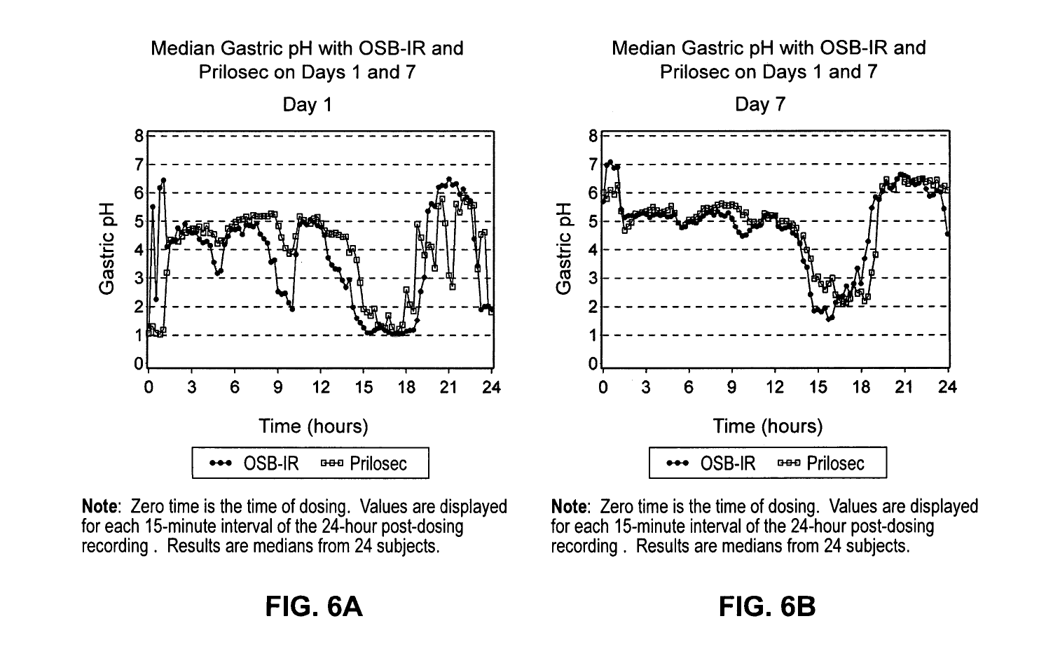

Median gastric pH for omeprazole plus sodium bicarbonate, baseline and for Prilosec® over time is illustrated in FIG. 6. On Day 1, there was an increase in median gastric pH during the first hour after dosing with omeprazole plus sodium bicarbonate, but not with Prilosec® (FIG. 6a). This reflected neutralization of gastric acid by the sodium bicarbonate in the omeprazole plus sodium bicarbonate treatment. FIG. 6a also shows that on Day 1 there was a greater decrease in gastric pH during each of three postprandial periods with omeprazole plus sodium bicarbonate than with Prilosec®. However, on Day 7 the time-course for median gastric pH with omeprazole plus sodium bicarbonate was the same as that with Prilosec® (FIG. 6b). In particular, there was no decrease in gastric pH below 4 for any of the three postprandial periods for either omeprazole plus sodium bicarbonate or Prilosec®.

The median percent time gastric pH was ≦4 was somewhat higher on Day 1 for omeprazole plus sodium bicarbonate than for Prilosec®, but on Day 7 they were the same, as shown in Table 6.E. below.

TABLE 6.E
Percent Time Gastric pH ≦ 4 During 24 Hours with
Omeprazole plus Sodium Bicarbonate and Prilosec ®

| | Time Gastric pH ≦ 4 (%) | |
|---|---|---|
| | 40 mg omeprazole plus 1680 mg | |
| Assessment | sodium bicarbonate | Prilosec ® (40 mg) |
| Baseline | 87 | 88 |
| | (80-93) | (75-92) |
| Day 1 | 53 | 43 |
| | (22-77) | (19-61) |
| Day 7 | 23 | 23 |
| | (12-46) | (16-43) |

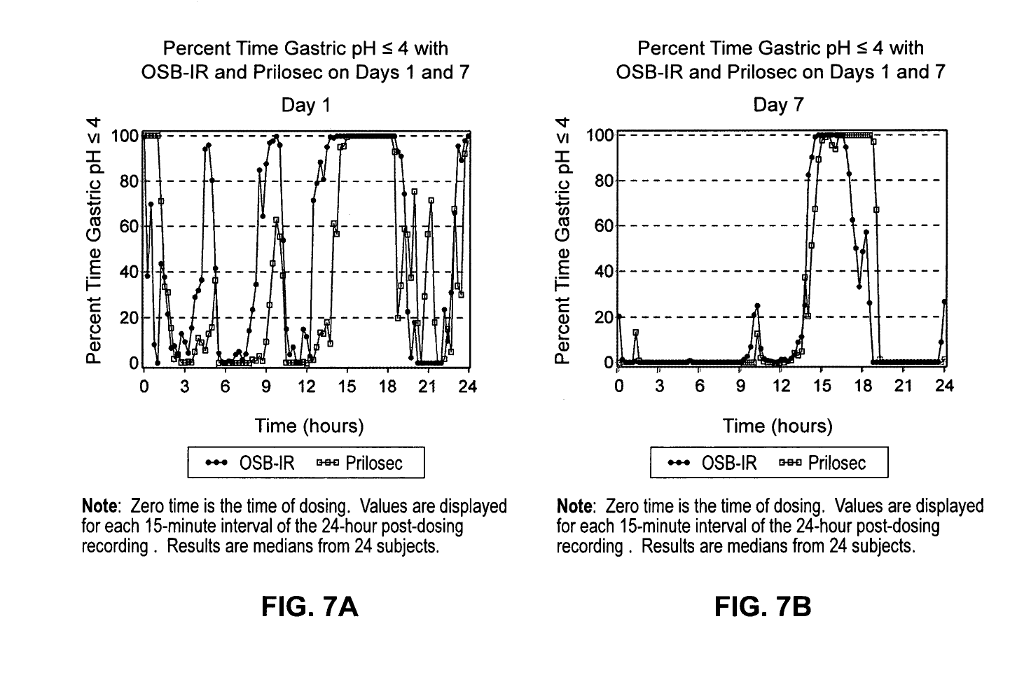

In FIG. 7a and FIG. 7b chart the amount of time gastric pH was ≦4 for omeprazole plus sodium bicarbonate and Prilosec® are plotted.

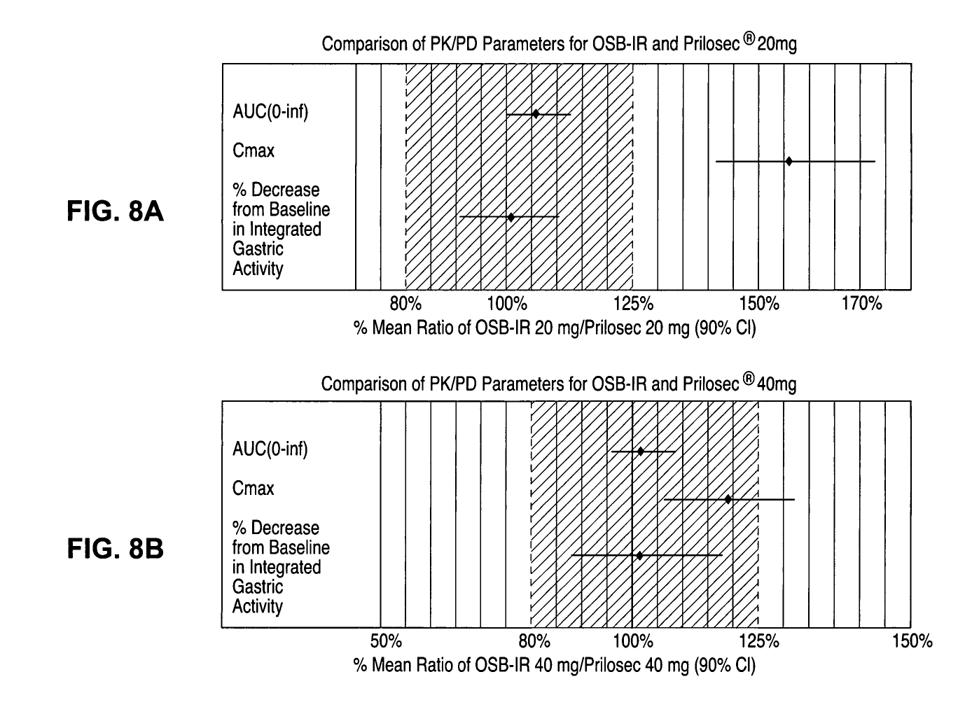

A summary comparison of pharmacokinetic and pharmacodynamic parameters between omeprazole (20 mg and 40 mg) plus sodium bicarbonate (1680 mg) and Prilosec® (20 mg and 40 mg) after 7 days is presented in FIG. 8a and FIG. 8b.

### Example 7

### Effect of Food Ingestion on Bioavailability of Omeprazole Plus Sodium Bicarbonate

This example describes studies indicating that food ingestion reduces bioavailability of omeprazole plus sodium bicarbonate, as compared to bioavailability when fasting. The studies were carried out as described in the OSB-IR-CO2 trial protocol. Subjects who received omeprazole plus sodium bicarbonate in Period 1 received an eighth dose omeprazole plus sodium bicarbonate given after a high fat meal.

Administration of 40 mg of omeprazole with 1680 mg of sodium bicarbonate at steady state one hour after initiation of a high fat meal reduced the bioavailability [AUC_{(0-inf)}] to 73% compared with administration after an overnight fast (pre-meal). The post-meal Cₘₐₓ was 40% of the pre-meal Cₘₐₓ. Food delayed the mean Tₘₐₓ by 55 minutes. Although there was a reduction in bioavailability of omeprazole plus sodium bicarbonate post-meal on Day 8 compared to pre-meal on Day 7, the Day 8 post-meal omeprazole plus sodium bicarbonate AUC_{(0-inf)} (3862 ng×hr/ml) was substantially greater than the pre-meal AUC_{(0-inf)} of omeprazole plus sodium bicarbonate or Prilosec® for all subjects on Day 1 (2228 and 2658 ng×hr/mL, respectively). The results are summarized in Table 7.A.

TABLE 7.A
Plasma Omeprazole Concentration
40 mg omeprazole plus 1680 mg sodium bicarbonate (Post-meal)

| | 40 mg omeprazole | | | 40 mg omeprazole | | | | |
|---|---|---|---|---|---|---|---|---|
| | plus 1680 mg | | | plus 1680 mg | | | | |
| | sodium bicarbonate | | | sodium bicarbonate | | | | |
| | (Post-meal) | | | (Pre-meal) | | | | % |
| | | Arithmetic | | | Arithmetic | | | Mean |
| Parameters | N | Mean | SD | N | Mean | SD | 90% Cl | Ratio |
| Cₘₐₓ | 16 | 880.6 | 378.7 | 16 | 2133 | 695.4 | — | — |
| (ng/mL) | | | | | | | | |
| Tₘₐₓ (hr) | 16 | 1.47 | 0.71 | 16 | 0.55 | 0.20 | — | — |
| AUC₍₀₋ₜ₎ | 16 | 3778 | 2700 | 16 | 4838 | 2643 | — | — |
| (ng × hr/mL) | | | | | | | | |
| AUC_{(0-inf)} | 16 | 3862 | 2874 | 16 | 4941 | 2849 | — | — |
| (ng × hr/mL) | | | | | | | | |
| In(Cₘₐₓ) | 16 | 6.68 | 0.52 | 16 | 7.59 | 0.43 | 34.9-46.5 | 40.2 |
| In[AUC₍₀₋ₜ₎] | 16 | 8.02 | 0.70 | 16 | 8.33 | 0.61 | 67.5-78.6 | 72.9 |
| In[AUC_{(0-inf)}] | 16 | 8.03 | 0.71 | 16 | 8.35 | 0.62 | 67.6-78.5 | 72.8 |

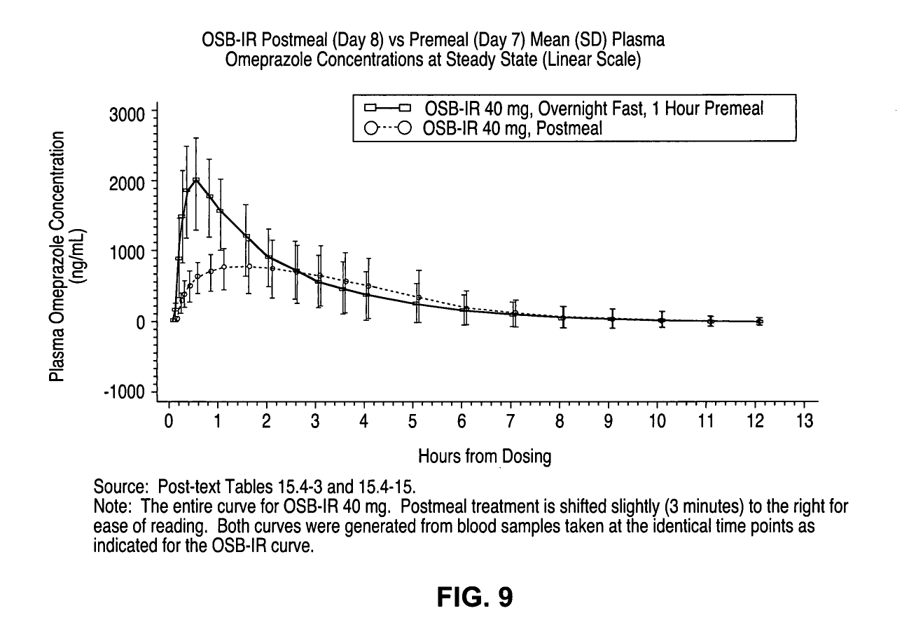

Mean plasma omeprazole concentrations at steady state for omeprazole plus sodium bicarbonate administration pre-meal (Day 7) and post-meal (Day 8) versus time plot are shown in FIG. 9.

### Example 8

### Extent and Duration of Increase in Gastric pH after Administration of Omeprazole Plus Sodium Bicarbonate

This example describes studies indicating that omeprazole plus antacid is effective at increasing and maintaining pH above 4.0 for several hours, and that increasing doses of omeprazole plus antacid increases the duration of acid suppression.

Pharmacodynamic parameters for administration of 40 mg omeprazole powder alone and 40 mg of omeprazole plus sodium bicarbonate were compared (SAN-15-CO1C). The results are summarized in Table 8.A.

TABLE 8.A
Pharmacodynamics of Omeprazole Powder (40 mg)
Administered With or Without Antacid (Pre-meal)

| | Number of | Median Integrated Gastric Acidity |
|---|---|---|
| | Subjects | 0-210 min. Post-meal (mmol × hr/L) |
| Control | 10 | 44 |
| Omeprazole Powder | 10 | 35 |
| Administered | | |
| 1 hour Pre-meal | | |
| Omeprazole Powder | 10 | 0.5 |
| Plus 30 mEq | | |
| Antacid Administered | | |
| 1 hour Pre-meal | | |

Omeprazole powder with antacid is considerably more effective in suppressing gastric acid, as compared to omeprazole powder alone (Table 8.B.).

FIG. 13 shows that a single pre-meal dose of 40 mg of omeprazole powder plus 30 mEq chewable antacid tablet given 30 minutes before a meal causes a greater decrease in gastric acidity (increased pH) and has a more prolonged suppressive effect on meal-induced acid secretion than Nexium® (study SAN-15-CO1B).

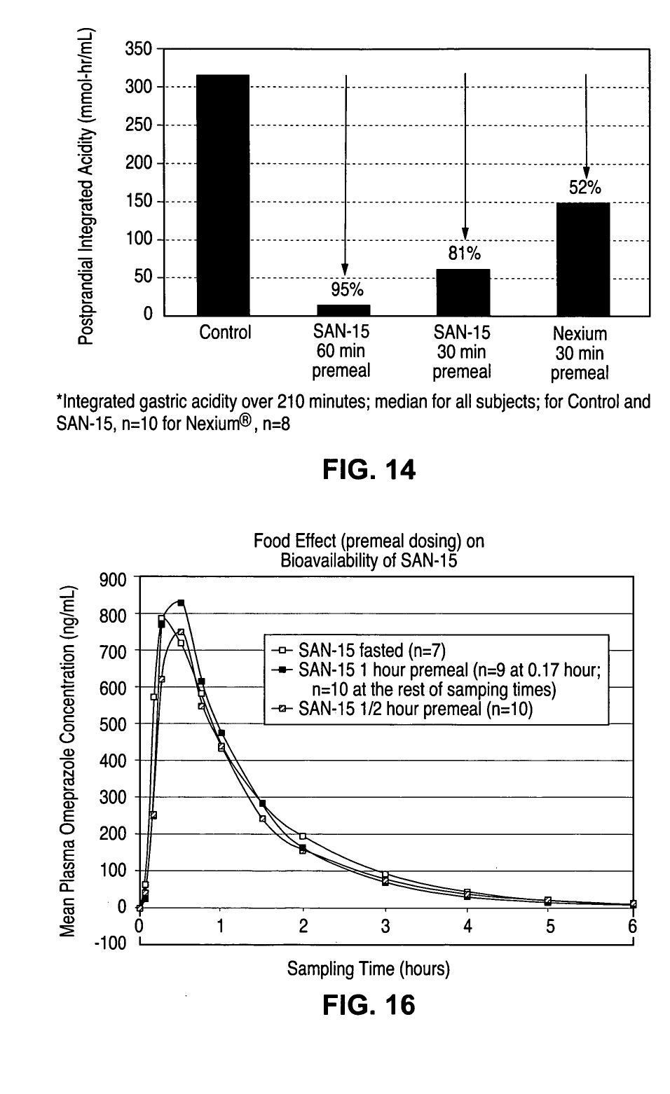

The data shown in FIG. 13 can also be analyzed as illustrated in FIG. 14. A single dose of 40 mg of omeprazole powder plus 30 mEq chewable antacid tablet administered 60 minutes pre-meal resulted in a 95% reduction in median gastric acidity over 210 minutes following a meal (study SAN-15-CO1B). A single dose of 40 mg of omeprazole powder plus 30 mEq antacid administered 30 minutes pre-meal resulted in an 81% reduction in median gastric acidity, while a single dose of Nexium® (40 mg) administered 30 minutes pre-meal resulted in only a 52% reduction in median gastric acidity. Thus, omeprazole/antacid is more effective than Nexium® in reducing integrated gastric acidity post-meal when administered pre-meal.

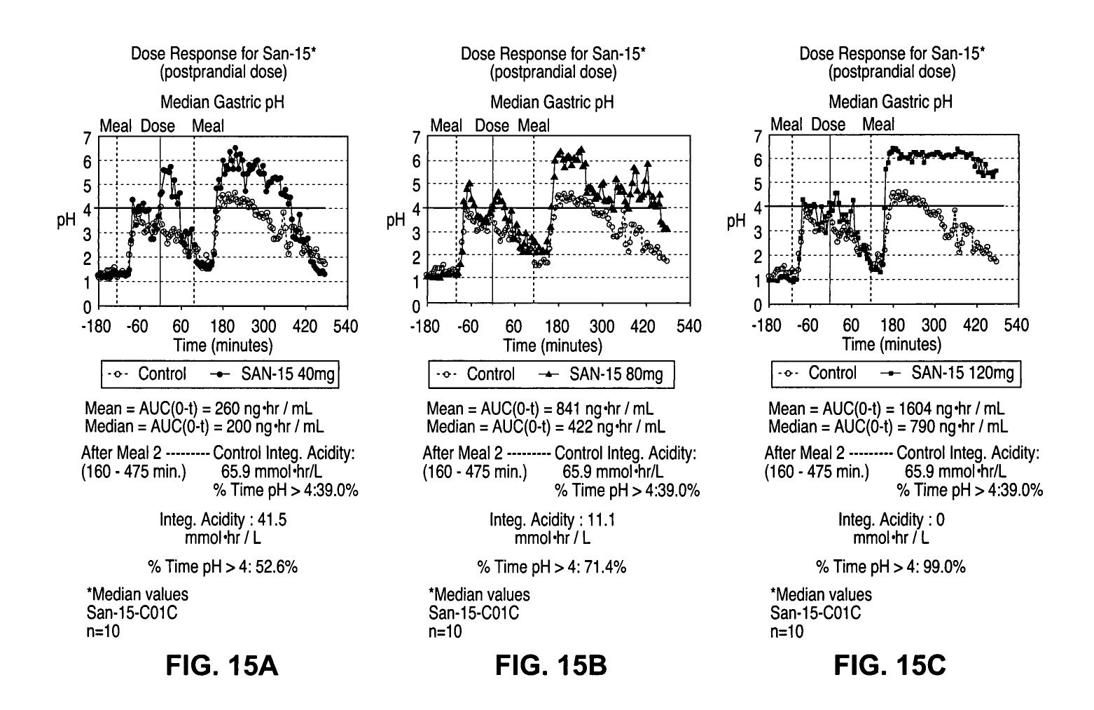

Study SAN-15-CO1C demonstrates that a single post-meal dose of 40 mg to 120 mg of omeprazole powder plus 30 mEq antacid given 90 minutes after breakfast is effective at increasing pH above 4.0 for 4-5 hours after lunch (FIG. 15(a)-15(c)). A dose-ranging effect with increasing amounts of omeprazole powder plus 30 mEq antacid was observed with regard to increase in acid suppression (FIGS. 15(a)-15(c)). The dose-ranging results in FIG. 15 are numerically summarized in Table 8.B.

TABLE 8.B
% Time pH > 4 After Ingestion of a Standard Lunch
With Administration of a Single Dose of Omeprazole Powder
plus Antacid 90 minutes After a Standardized Breakfast

| | Median Integrated | Median % |
|---|---|---|
| | Acidity mmol × hr/L | Time pH > 4 |
| Control | 65.9 | 39.0% |
| 40 mg of omeprazole powder | 41.5 | 52.6% |
| administered with antacid | | |
| 80 mg of omeprazole powder | 11.1 | 71.4% |
| administered with antacid | | |
| 120 mg of omeprazole powder | 0 | 99.0% |
| administered with antacid | | |

### Example 9

### Effect of Multiple Doses of Omeprazole Plus Sodium Bicarbonate on Bioavailability and Suppression of Gastric Acidity

This example describes studies indicating that omeprazole plus sodium bicarbonate delivered multiple times exhibits increased bioavailability and increased and sustained supression of gastric acidity. To evaluate omeprazole pharmacokinetics (plasma omeprazole) and pharmacodynamics (gastric pH and integrated gastric acidity) for multiple dose administrations, studies were performed as described in the OSB-IR-CO2, OSB-IR-CO5 and OSB-IR-CO6 trial protocols.

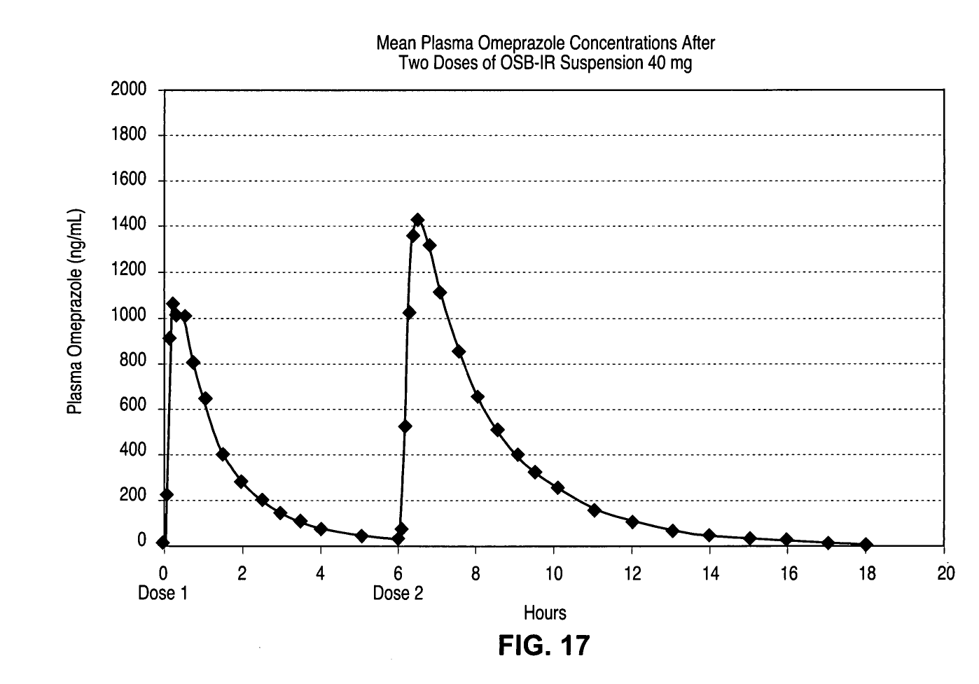

Plasma omeprazole following two doses of 40 mg OSB-IR administered six hours apart is illustrated in FIG. 17 (OSB-IR-CO5). These results indicate that a subsequent omeprazole administration can exhibit greater bioavailability than a prior adminstration.

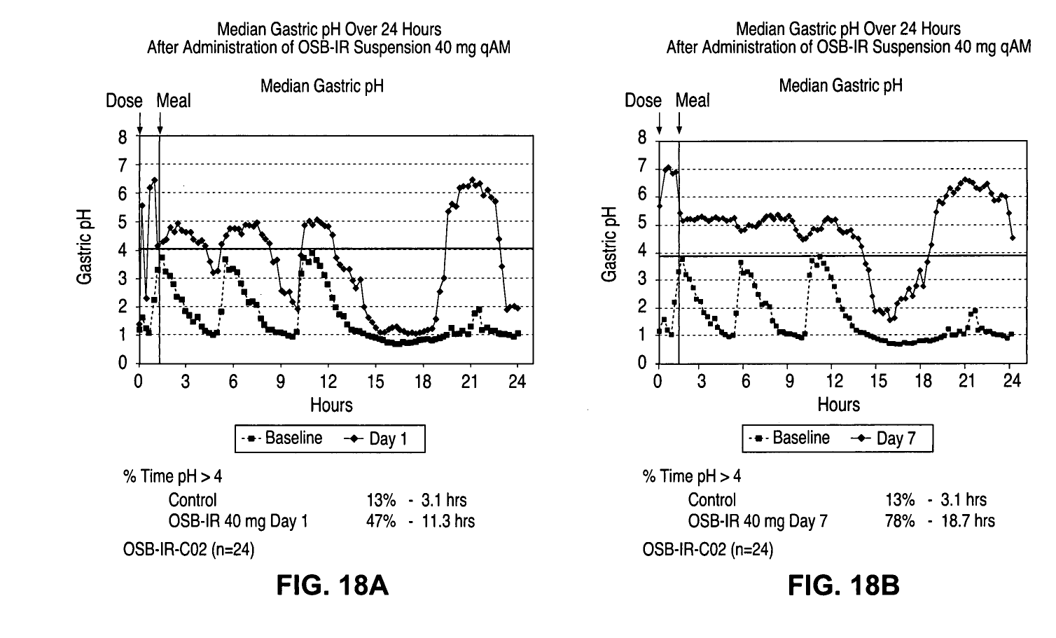

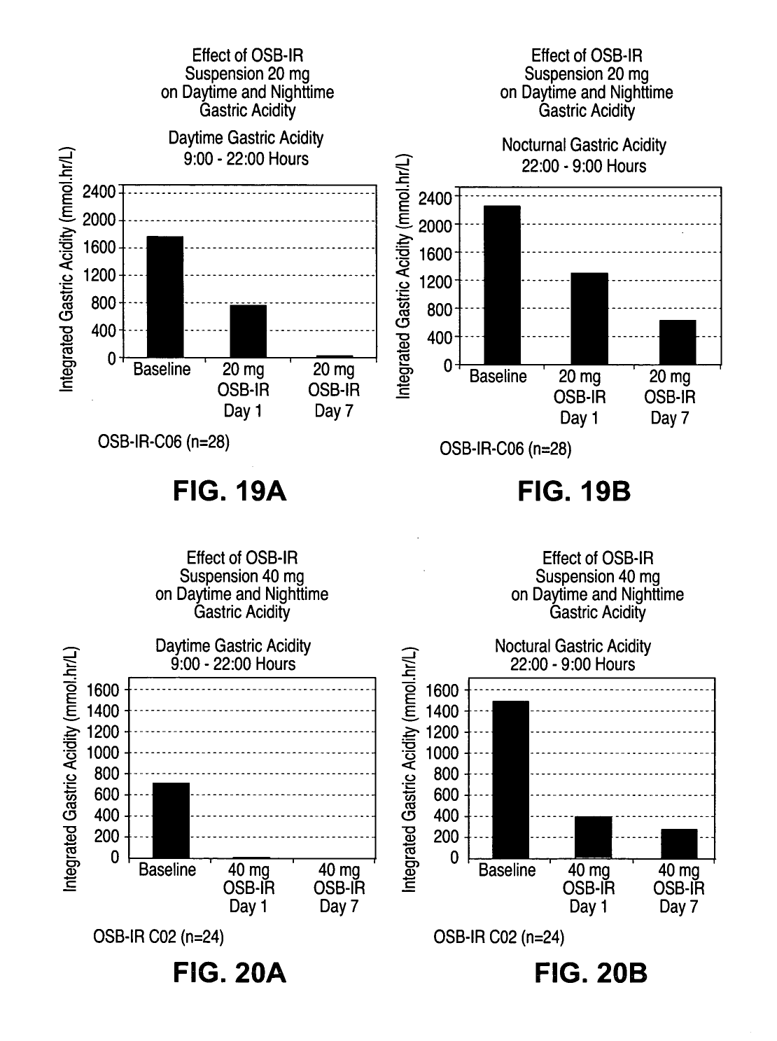

As demonstrated in FIG. 2 and FIG. 3, plasma levels and systematic exposure of omeprazole from 40 mg omeprazole plus antacid increases from a single dose to 7 days of once-daily dosing. The duration of median gastric pH increase over baseline was greater on day 7 as compared to day 1 (FIG. 18a vs. FIG. 18b). At day 7, throughout most of the day the pH was >4. FIG. 19 and FIG. 20 illustrate daytime (9:00 to 22:00 hours) gastric activity versus nocturnal (22:00 to 9:00 hours) gastric acidity for the 20 mg and 40 mg doses of omeprazole (plus antacid). The results in FIG. 19 and FIG. 20 indicate that the median integrated gastric acidity increases over baseline during the day as well as in the evening (nocturnal) when baseline gastric acidity typically is greatest. This data also indicate that there is a greater suppression of gastric acidity on day 7 as compared with that on day 1.

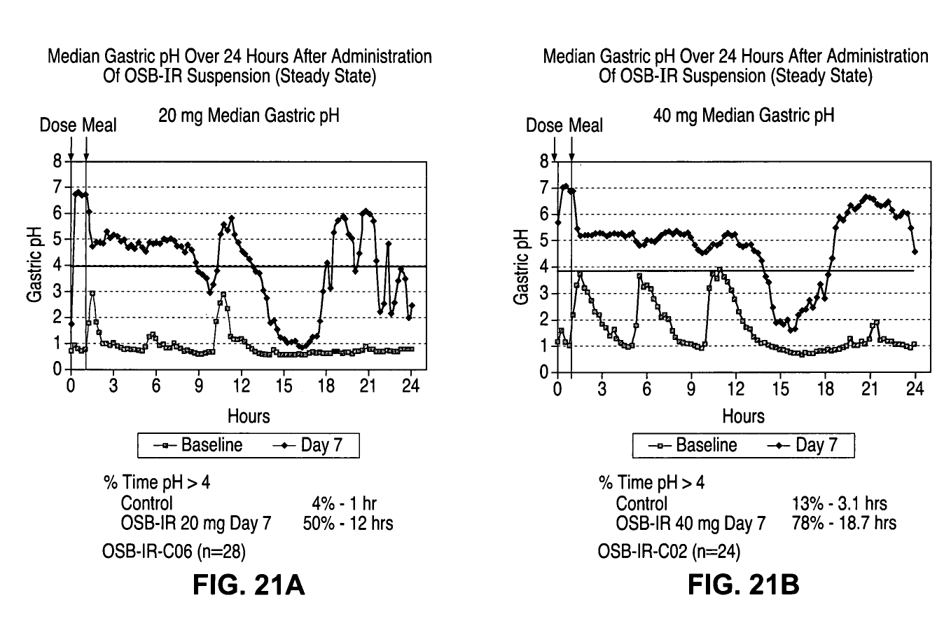

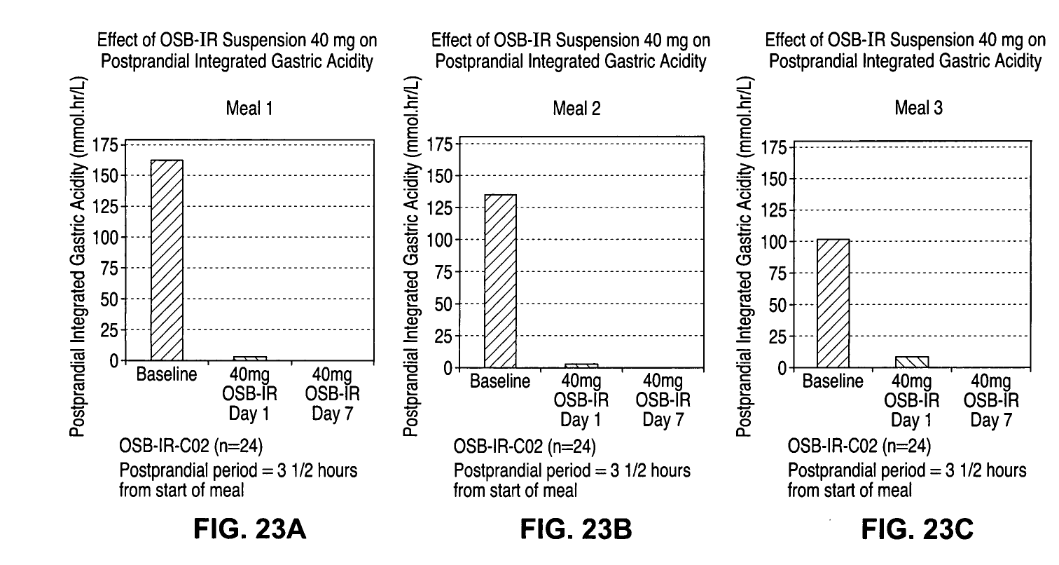

As illustrated in FIG. 21 and FIG. 23, the median gastric pH is greater as the dose of omeprazole (delivered with antacid) is increased. For example, a greater cumulative effect at 40 mg dose than at 20 mg dose was observed (compare FIG. 21a and FIG. 21b). However, the supressive effect of the 20 mg dose is still present throughout the day and evening.

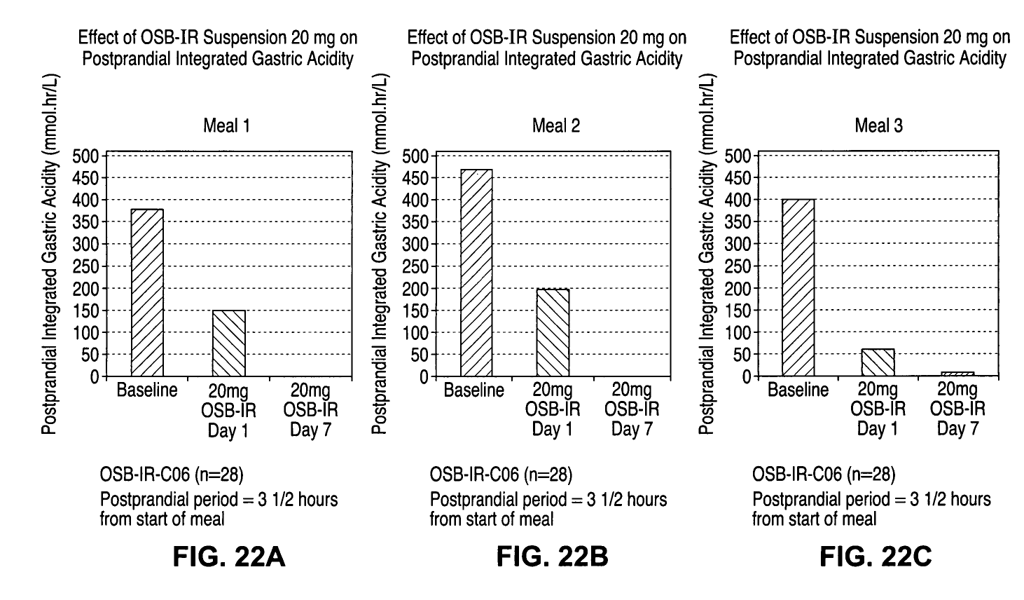

FIG. 22 and FIG. 23 present the effects of omeprazole 20 mg and omeprazole 40 mg, respecivly, on postprandial (post-meal) gastric acidity. There is a dose-related decrease in integrated gastric acidity, and this effect is greater after 7 days of once-daily doses than on day 1.

As illustrated in the foregoing figures, repeated once-daily doses of omeprazole plus antacid over time provided a cumulative reduction in gastric acidity having a duration extending throughout the day and evening. Because of the observed cumulative effect following meal consumption, repeated doses of omeprazole plus antacid may be useful in reducing or preventing the occurrence (frequency), duration or severity of meal-induced heartburn.

### Example 10

### Effect of Omeprazole on Nocturnal Acid Breakthrough

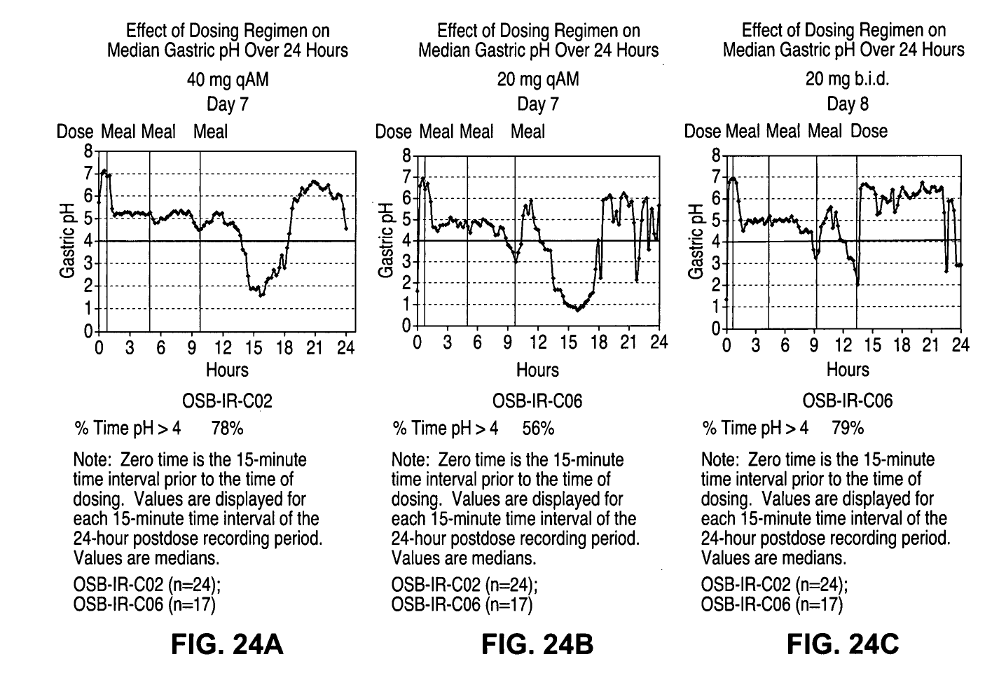

This example describes study OSB-IR-CO6 indicating that a 20 mg dose of omeprazole with antacid prior to bedtime, after repeated once-daily omeprazole doses, can supress nocturnal gastric acidity (FIG. 24(b) and FIG. 24(c)). Also, illustrated in FIG. 24(a) to FIG. 24(c) is that two 20 mg doses (one at bedtime) of omeprazole plus antacid are better than one 40 mg dose in the morning in suppressing nighttime gastric acidity. The results demonstrate that omeprazole with antacid administered prior to bedtime may be useful in treating one or more symptoms associated with nocturnal gastric acidity, such as nocturnal heartburn.

### Example 11

### Effect of Omeprazole on Upper GI Bleeding

This example describes a study (OSB-IR-CO3) indicating that a 40 mg daily dose of omeprazole with antacid prevented or reduced upper GI bleeding in critically ill patients, and was not inferior to cimetidine in preventing or reducing upper GI bleeding (FIG. 28) [cimetidine is the only FDA-approved drug for prevention of UGI bleeding in critically ill patients].

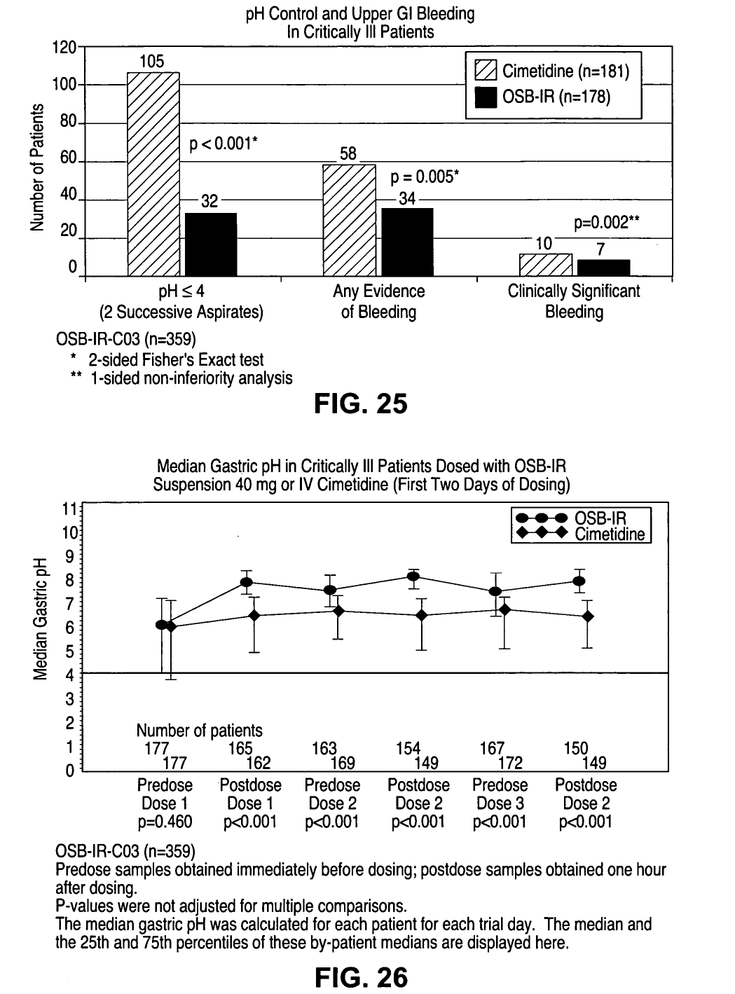

As illustrated in FIG. 25, the results indicate that fewer patients had gastric aspirates with a pH less than 4 in the OSB-IR group than in the cimetidine group. Fewer patients treated with OSB-IR suspension exhibited bleeding (both any evidence and clinically significant amounts) than in the cimetidine treated group.

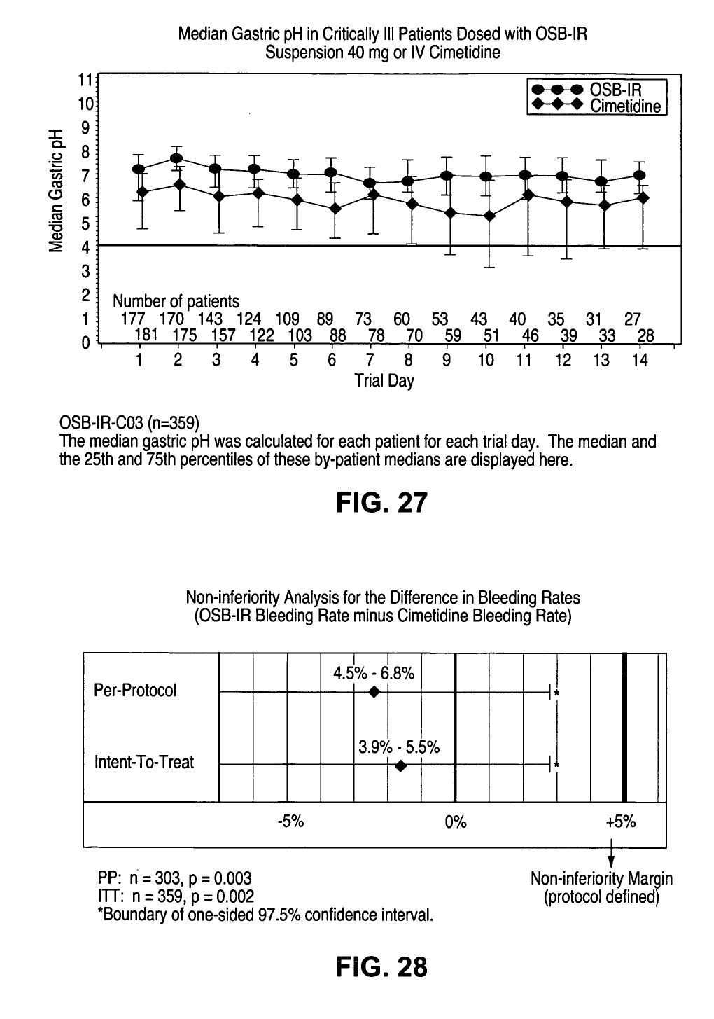

The results in FIG. 26 illustrate median gastric pH of critically ill patients treated over the first 2 days, and indicate that OSB-IR (40 mg omeprazole) provided a statistically significantly greater increase in gastric pH in OSB-IR patients than in the cimetidine patients. The results in FIG. 27 illustrate median gastric pH for each of the 14 days of the study, and indicate that OSB-IR (40 mg omeprazole) provided a statistically significantly greater increase in pH on all study days in the OSB-IR patients than in the cimetidine patients.

The invention has been described in an illustrative manner, and it is to be understood the terminology used is intended to be in the nature of description rather than of limitation. All patents and other references cited herein are incorporated herein by reference in their entirety. Obviously, many modifications, equivalents, and variations of the present invention are possible in light of the above teachings. Therefore, it is to be understood that within the scope of the appended claims, the invention may be practiced other than as specifically described.

What is claimed is:

1. A pharmaceutical composition comprising:
(a) an amount of at least one acid labile proton pump inhibitor; and
(b) at least one buffering agent in an amount sufficient to inhibit or reduce degradation of at least some of the proton pump inhibitor,
wherein the composition is administered to a subject prior to a meal and is in an amount effective to maintain gastric pH greater than about 4.0 for at least about 1 hour following the meal.

2. The pharmaceutical composition of claim 1, wherein the composition is in an amount effective to increase the gastric pH of the subject to at least about 3 prior to the meal.

3. The pharmaceutical composition of claim 1, wherein the composition is in an amount effective to increase the gastric pH of the subject to at least about 3 prior within 30 minutes after administration.

4. The pharmaceutical composition of claim 1, wherein a therapeutically effective amount of the proton pump inhibitor is absorbed within about 1 hour after administration.

5. The pharmaceutical composition of claim 1, wherein at least some of the proton pump inhibitor is not enteric-coated.

6. The pharmaceutical composition of claim 1, wherein the composition is in an amount effective to maintain gastric pH greater than about 4.5 for at least about 1 hour following the meal.

7. The pharmaceutical composition of claim 1, wherein the maximum pH is reached within about 30 minutes after administration of the composition.

8. The pharmaceutical composition of claim 1, wherein the maximum pH is reached within about 15 minutes after administration of the composition.

9. The pharmaceutical composition of claim 1, wherein the gastric pH is greater than 4.0 at least about 50% of a time period up to seven hours.

10. The pharmaceutical composition of claim 1, wherein the gastric pH is greater than 4.0 at least about 75% of a time period up to seven hours.

11. The pharmaceutical composition of claim 1, wherein the amount of proton pump inhibitor is about 5 to about 500 mg.

12. The pharmaceutical composition of claim 1, wherein the proton pump inhibitor is about 10 mg.

13. The pharmaceutical composition of claim 1, wherein the proton pump inhibitor is about 20 mg.

14. The pharmaceutical composition of claim 1, wherein the proton pump inhibitor is about 40 mg.

15. The pharmaceutical composition of claim 1, wherein the proton pump inhibitor is about 80 mg.

16. The pharmaceutical composition of claim 1, wherein the proton pump inhibitor is selected from the group consisting of omeprazole, hydroxyomeprazole, esomeprazole, tenatoprazole, lansoprazole, pantoprazole, rabeprazole, dontoprazole, habeprazole, perprazole, ransoprazole, pariprazole, leminoprazole; or a free base, free acid, salt, hydrate, ester, amide, enantiomer, isomer, tautomer, polymorph, derivative, or prodrug thereof.

17. The pharmaceutical composition of claim 1, wherein the proton pump inhibitor comprises omeprazole, or a free base, free acid, salt, hydrate, ester, amide, enantiomer, isomer, tautomer, polymorph, derivative, or prodrug thereof.

18. The pharmaceutical composition of claim 1, wherein the proton pump inhibitor comprises lansoprazole, or a free base, free acid, salt, hydrate, ester, amide, enantiomer, isomer, tautomer, polymorph, derivative, or prodrug thereof.

19. The pharmaceutical composition of claim 1, wherein the proton pump inhibitor comprises esomeprazole, or a free base, free acid, salt, hydrate, ester, amide, enantiomer, isomer, tautomer, polymorph, derivative, or prodrug thereof.

20. The pharmaceutical composition of claim 1, wherein the at least about 50% of total area under a serum concentration time curve (AUC) for the proton pump inhibitor occurs within about 1.75 hours after administration of a single dose of the composition to the subject.

21. The pharmaceutical composition of claim 1, wherein the at least about 50% of total area under a serum concentration time curve (AUC) for the proton pump inhibitor occurs within about 1.0 hour after administration of a single dose of the composition to the subject.

22. The pharmaceutical composition of claim 1, wherein the proton pump inhibitor is encapsulated with a material that enhances the shelf-life of the pharmaceutical composition.

23. The pharmaceutical composition of claim 1, wherein the buffering agent is selected from the group consisting of an amino acid, an alkali metal salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, and mixtures thereof.

24. The pharmaceutical composition of claim 1, wherein the buffering agent is selected from sodium bicarbonate, calcium carbonate, magnesium hydroxide, and mixtures thereof.

25. The composition of claim 1, wherein the buffering agent is present in an amount from about 0.1 mEq/mg to about 5 mEq/mg of the proton pump inhibitor.

26. The composition of claim 1, wherein the buffering agent is present in an amount from about 0.4 mEq/mg to about 1.5 mEq/mg of the proton pump inhibitor.

27. The composition of claim 1 comprising from about 200 to about 2000 mg of buffering agent.

28. The pharmaceutical composition of claim 1, wherein the composition is in the form of a powder, a tablet, a bite-disintegration tablet, a chewable tablet, a caplet, a capsule, an effervescent powder, a rapid-disintegration tablet, or an aqueous suspension or emulsion.

29. A pharmaceutical composition of claim 1, wherein at least some of the proton pump inhibitor is microencapsulated.

30. A pharmaceutical compositin of claim 1, wherein at least some of the proton pump inhibitor is micronized.

31. A pharmaceutical composition of claim 1, wherein at least some of the proton pump inhibitor is coated.

32. The pharmaceutical composition of claim 1, further comprising an excipient.

33. The pharmaceutical composition of claim 32, wherein said excipient is selected from the group consisting of a parietal cell activator, erosion facilitator, flavoring agent, sweetening agent, diffusion facilitator, antioxidant and a carrier material selected from a binder, suspending agent, disintegration agent, filling agent, surfactant, solubilizer, stabilizer, lubricant, wetting agent, diluent, anti-adherent, and antifoaming agent.

34. A pharmaceutical composition comprising:
(a) an amount of at least one acid labile proton pump inhibitor; and
(b) at least one buffering agent in an amount sufficient to inhibit or reduce degradation of at least some of the proton pump inhibitor,
wherein the composition is administered to a subject before a meal and causes a increase in gastric pH to at least about 3.0 within about 30 minutes after administration.

35. The pharmaceutical composition of claim 34, wherein a therapeutically effective amount of the proton pump inhibitor is absorbed within about 1 hour after administration of the composition.

36. A pharmaceutical composition comprising:
(a) a therapeutically effective amount of at least one acid labile proton pump inhibitor; and
(b) at least one buffering agent in an amount sufficient to inhibit or reduce degradation of at least some of the proton pump inhibitor by gastric fluid, wherein the composition is in an amount effective to reduce or inhibit upper GI bleeding following administration to the subject.

37. The composition of claim 36, wherein the pharmaceutical composition is in a liquid form and reduces mortality or nosocomial pneumonia due to upper GI bleeding, or a complication associated with upper GI bleeding.

38. A method of administering a compound according to claim 1 for the treatment of a gastric acid related disorder.

39. The method according to claim 38, wherein the gastric acid related disorder is duodenal ulcer disease, gastric ulcer disease, gastroesophageal reflux disease, erosive esophagitis, poorly responsive symptomatic gastroesophageal reflux disease, pathological gastrointestinal hypersecretory disease, Zollinger Ellison syndrome, heartburn, esophageal disorder, or acid dyspepsia.

40. A method of preventing or inhibiting breakthrough of pH control in a subject by administering a compound according to claim 1, wherein the subject has previously been administered a compound within about the past 2-22 hours that increases gastric pH to above 3, thereby preventing or inhibiting breakthrough of pH control.

41. The method of claim 40, wherein the composition is administered before retiring to bed.

42. The method of claim 40, wherein the composition is administered to treat or prevent nocturnal heartburn.

43. The method of claim 40, wherein integrated gastric acidity of the subject is reduced by at least about 25-500%.

44. A method of rapidly reducing production of gastric acid in a subject by administering a composition according to claim 1.

45. A method of treating a gastric acid related disorder induced by a meal by administering a composition according to claim 1 prior to the meal, wherein the amount of proton pump inhibitor is effective to reduce or inhibit one or more symptoms of the gastric acid related disorder in the subject.

46. A method of treating a gastric acid related disorder induced by a meal in a subject by administering to the subject within about 4 hours following ingestion of the meal a composition comprising,
(a) at least one acid labile proton pump inhibitor; and
(b) at least one buffering agent in an amount sufficient to inhibit or reduce degradation of at least some of the proton pump inhibitor,
wherein the amount of proton pump inhibitor is effective to reduce or inhibit one or more symptoms of the gastric acid related disorder in the subject.

47. A method of treating a critically ill subject having or at risk of having upper GI bleeding or a symptom associated with upper GI bleeding comprising administering to the subject a liquid formulation comprising:
(a) at least one acid labile proton pump inhibitor; and
(b) at least one buffering agent in an amount sufficient to inhibit or reduce degradation of at least some of the proton pump inhibitor,
wherein the amount of proton pump inhibitor is effective to reduce or inhibit upper GI bleeding or the symptom associated with upper GI bleeding in the critically ill subject.

48. The method of claim 47, wherein the subject has a nasogastric (NG) tube or a gastric tube.

49. The method of claim 47, wherein the incidence, severity, duration or frequency of upper GI bleeding or one or more symptoms associated with upper GI bleeding is reduced in the subject.

50. The method of claim 47, wherein clinically significant bleeding is reduced in the critically ill subject.

51. The method of claim 47, wherein mortality or nosocomial pneumonia associated with upper GI bleeding is reduced in the critically ill subject.

52. A method of treating a subject having or at risk of having a gastric acid related disorder, said subject having difficulty swallowing a pill, capsule or tablet by administering a pharmaceutical composition according to claim 1, wherein the composition is administered in a liquid form.

53. A method for treating heartburn by administering a pharmaceutical composition according to claim 1.
